(19) United States
(12) Patent Application Publication
(10) Pub. No.: US 2022/0267716 A1
(43) Pub. Date: Aug. 25, 2022

(54) METHODS AND COMPOSITIONS FOR CULTURING HEMOGLOBIN-DEPENDENT BACTERIA

(71) Applicant: Evelo Biosciences, Inc., Cambridge, MA (US)

(72) Inventors: Valeria Kravitz, Norwood, MA (US); Maria Sizova, Roslindale, MA (US)

(21) Appl. No.: 17/632,449

(22) PCT Filed: Jul. 31, 2020
(86) PCT No.: PCT/US20/44378

§ 371 Date: Feb. 2, 2022

Related U.S. Application Data
(60) Provisional application No. 62/971,391, filed on Feb. 7, 2020.
(60) Provisional application No. 62/898,372, filed on Sep. 10, 2019.
(60) Provisional application No. 62/882,021, filed on Aug. 2, 2019.

(51) Int. Cl. C12N 1/20; C12M 1/00; C12M 1/26

(52) U.S. Cl. CPC C12N 1/20; C12M 29/20; C12M 33/10; C12N 2500/02; C12N 2500/92; C12N 2502/70; C12N 2500/74

(57) ABSTRACT

Provided herein are methods and compositions related to culturing hemoglobin-dependent bacteria.

### CROSS-REFERENCE TO RELATED APPLICATIONS

This application claims the benefit of U.S. Provisional Application No. 62/882,021, filed on Aug. 2, 2019; U.S. Provisional Application No. 62/898,372, filed on Sep. 10, 2019; and U.S. Provisional Application No. 62/971,391, filed on Feb. 7, 2020; the entire contents of each of said applications are incorporated herein in their entirety by this reference.

### BACKGROUND

The composition of a person's microbiome can play an important role in their health and well-being. Indeed, disruption of an individual's microbiome has been implicated in numerous diseases, including inflammatory bowel diseases, immune disorders, type 2 diabetes, neurodegenerative disorders, cardiovascular diseases, and cancers. Thus, microbiome modulation is an attractive therapeutic strategy for such diseases.

One way to modulate a person's microbiome is by orally administering to them one or more strains of beneficial bacteria. However, development of such therapies have been hindered by the fact that large-scale production of many bacterial strains has proven challenging, particularly for bacterial strains that require hemoglobin (or its derivatives such as hemin) for growth.

Hemoglobin is an iron-containing metalloprotein in red blood cells that captures atmospheric oxygen in the lungs and carries it to the rest of the body. Iron is an essential nutrient for almost all forms of life, including bacteria. As hemoglobin is the most abundant reservoir of iron within humans, much of the bacteria that make up the human microbiome use hemoglobin or its derivatives as their primary source of iron. Often, such hemoglobin-dependent bacteria require the presence of hemoglobin or hemin for optimal in vitro growth. However, commercial hemoglobin and its derivatives are typically purified from animal sources, such as from porcine blood, which results in purified hemoglobin being costly. Moreover, the animal sourcing of hemoglobin can raise ethical and/or religious objections among certain groups. Finally, GMP (good manufacturing practice)-grade hemoglobin is not easily sourced, making the large-scale manufacture of hemoglobin-dependent bacteria for pharmaceutical purposes particularly challenging.

Accordingly, there is a great need for compositions and methods that enable the optimal growth of hemoglobin-dependent bacteria in the absence of hemoglobin, its derivatives, or any other animal-derived components.

### SUMMARY

As demonstrated herein, certain hemoglobin substitutes, such as cyanobacteria (including cyanobacteria-comprising biomasses) and/or cyanobacteria-derived components, can be used instead of hemoglobin to facilitate the growth of hemoglobin-dependent bacteria in culture. The hemoglobin substitutes provided herein support the growth of hemoglobin-dependent bacteria in the absence of hemoglobin or a derivative thereof and/or with use of reduced amounts of hemoglobin or a derivative thereof.

For example, as demonstrated herein, spirulina and/or certain spirulina-derived components (e.g., soluble spirulina components) can be used in place of hemoglobin in growth media to facilitate the in vitro culturing of otherwise hemoglobin-dependent bacteria, including bacteria of the genus Prevotella (such as Prevotella histicola), bacteria of the genus Faecalibacterium, bacteria of the genus Fournierella, bacteria of the genus Parabacteroides, bacteria of the genus Bacteroides, and bacteria of the genus Allistipes. Spirulina is a biomass of Arthrospira platensis and/or Arthrospira maxima cyanobacteria that has been consumed by humans for centuries in Mexico and some African countries. More recently, spirulina has been recognized as a rich source of proteins and many nutrients, and is therefore commonly consumed as a nutritional supplement. As spirulina is relatively inexpensive, vegetarian, kosher, and readily available at GMP-grade, it is an attractive alternative to hemoglobin in bacterial cell culture applications.

In certain aspects, provided herein are methods and compositions that allow for the culturing of hemoglobin-dependent bacteria in the absence of hemoglobin, hemoglobin derivatives, and/or, in certain embodiments, any animal products. Growth of hemoglobin-dependent bacteria in the absence of hemoglobin is accomplished through the inclusion in the cell culture media of certain hemoglobin substitutes provided herein. In certain embodiments, the hemoglobin substitute is a cyanobacteria (e.g., cyanobacteria of the genus Arthrospira, such as Arthrospira platensis and/or Arthrospira maxima) that is able to substitute for hemoglobin to support growth of otherwise hemoglobin-dependent bacteria.

In certain embodiments, the hemoglobin substitute is a biomass of cyanobacteria (e.g., spirulina) that is able to substitute for hemoglobin to support growth of otherwise hemoglobin-dependent bacteria. In certain embodiments, the hemoglobin substitute is a component of cyanobacteria (e.g., a component of cyanobacteria of the genus Arthrospira, such as Arthrospira platensis and/or Arthrospira maxima) (e.g., a soluble component thereof) that is able to substitute for hemoglobin to support growth of otherwise hemoglobin-dependent bacteria. In some embodiments, the hemoglobin substitute is a green algae that is able to substitute for hemoglobin to support growth of otherwise hemoglobin-dependent bacteria. In certain embodiments, the hemoglobin substitute is a component (e.g., a soluble component) of green algae that is able to substitute for hemoglobin to support growth of otherwise hemoglobin-dependent bacteria.

Thus, in certain aspects, provided herein are methods and compositions for culturing hemoglobin-dependent bacteria in growth media that includes a hemoglobin substitute provided herein. In some aspects, provided herein are compositions (e.g., growth media) comprising a hemoglobin substitute provided herein that are useful for culturing hemoglobin-dependent bacteria in conditions free of hemoglobin or derivatives thereof, as well as methods of making and/or using such compositions.

In some embodiments, the hemoglobin substitute used in the methods and compositions provided herein is spirulina or components thereof (i.e., spirulina components able to substitute for hemoglobin to support growth of otherwise hemoglobin-dependent bacteria, such as a soluble spirulina component). For example, provided herein are methods and compositions for culturing hemoglobin-dependent bacteria in growth media that includes spirulina or components thereof (e.g., a soluble component thereof). In some aspects, provided herein are compositions (e.g., growth media) comprising spirulina or components thereof that are useful for culturing hemoglobin-dependent bacteria in conditions free of hemoglobin or derivatives thereof, as well as methods of making and/or using such compositions. In some embodiments, the component of spirulina comprises Chlorophyll A.

In certain aspects, provided herein is a growth medium for use in culturing hemoglobin-dependent bacteria, the growth medium comprising a hemoglobin substitute provided herein (e.g., spirulina or a component thereof). In some embodiments, the growth medium comprises hemoglobin-dependent bacteria. In certain embodiments, provided herein is a hemoglobin substitute provided herein (e.g., spirulina or a component thereof) for use as a substitute for hemoglobin or a derivative thereof in a growth medium for hemoglobin-dependent bacteria.

In certain aspects, provided herein is a method of culturing hemoglobin-dependent bacteria, the method comprising incubating the hemoglobin-dependent bacteria in a growth medium that comprises a hemoglobin substitute provided herein (e.g., spirulina or a component thereof) (e.g., in the absence of hemoglobin or a derivative thereof). In some aspects, provided herein is a method of culturing hemoglobin-dependent bacteria, the method comprising (a) adding a hemoglobin substitute provided herein (e.g., spirulina or a component thereof) and hemoglobin-dependent bacteria to a growth medium; and (b) incubating the hemoglobin-dependent bacteria in the growth medium.

In certain aspects, provided herein is a bacterial composition comprising a growth medium comprising a hemoglobin substitute provided herein (e.g., spirulina or a component thereof) and hemoglobin-dependent bacteria.

In certain aspects, provided herein is a bioreactor comprising hemoglobin-dependent bacteria in a growth medium comprising a hemoglobin substitute provided herein (e.g., spirulina or a component thereof). In some embodiments, provided herein is a method of culturing hemoglobin-dependent bacteria, the method comprising comprises incubating the hemoglobin-dependent bacteria in a bioreactor provided herein.

In some embodiments, the growth medium comprises spirulina. In some embodiments, the growth medium comprises at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of spirulina. In some embodiments, the growth medium comprises at least 1 g/L and no more than 2 g/L of spirulina. In some embodiments, the growth medium comprises about 1 g/L of spirulina. In some embodiments, the growth medium comprises about 2 g/L of spirulina. In some embodiments, the growth medium comprises yeast extract, soy peptone A2SC 19649, Soy peptone E110 19885, dipotassium phosphate, monopotassium phosphate, L-cysteine-HCl, ammonium chloride, glucidex 21 D, and/or glucose. In some embodiments, the growth media comprises about 5 g/L glucose, about 10 g/L yeast extract 19512, about 10 g/L soy peptone A2 SC 19649, about 10 g/L soypeptone E110 19885, about 2.5 g/L dipotassium phosphate K2HPO4, and about 0.5 g/L L-cysteine-HCl. In some embodiments, the growth medium is at a pH of 5.5 to 7.5. In certain embodiments, the growth medium is at a pH of about 6.5. In some embodiments of the methods and compositions provided herein, the growth medium does not comprise hemoglobin or a derivative thereof. In certain embodiments, the growth medium does not comprise animal products.

In some embodiments, the hemoglobin substitute used in the methods and compositions provided herein is a cyanobacteria, a cyanobacteria biomass and/or a cyanobacteria component (i.e., a cyanobacteria, cyanobacteria biomass, and/or cyanobacteria component able to substitute for hemoglobin to support growth of otherwise hemoglobin-dependent bacteria). In certain embodiments, any cyanobacteria, cyanobacteria biomass, or cyanobacteria component that is capable of functioning as a hemoglobin substitute can be used in the methods and compositions provided herein. In certain embodiments, the cyanobacteria is of the order Oscillatoriales. In some embodiments, the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema. In some embodiments, the cyanobacteria is Arthrospira platensis and/or Arthrospira maxima. In some embodiments, the cyanobacteria is spirulina.

In some embodiments, the hemoglobin substitute used in the methods and compositions provided herein is a green algae, a green algae biomass and/or a green algae component (i.e., a green algae, green algae biomass and/or green algae component able to substitute for hemoglobin to support growth of otherwise hemoglobin-dependent bacteria). In certain embodiments, any green algae, green algae biomass, or a green algae component that is capable of functioning as a hemoglobin substitute can be used in the methods and compositions provided herein. In certain embodiments, the green algae is of the order Chlorellales. In some embodiments, the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

In some embodiments of the methods and compositions provided herein, the hemoglobin-dependent bacteria can be any bacteria that require the presence of hemoglobin or a hemoglobin derivative for optimal growth (i.e., for optimal growth in the absence of spirulina or a component thereof provided herein). In some embodiments of the methods and compositions provided herein, the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella. In some embodiments, the hemoglobin-dependent bacteria are of the genus Prevotella. In some embodiments, the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis. In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptomphllus lacrimalis, Rarimicrobium hominis, Shuttleworthia satelles, or Turicibacter sanguinis.

In some embodiments of the methods and compositions provided herein, the hemoglobin-dependent bacteria are a strain of the species Prevotella histicola. In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain B 50329. In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain B 50329 (NRRL accession number B 50329). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain B 50329 (NRRL accession number B 50329). In certain embodiments, the Prevotella histicola strain is Prevotella Strain B 50329 (NRRL accession number B 50329).

In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain C (ATCC Deposit Number PTA-126140, deposited on Sep. 10, 2019). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain C (PTA-126140). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain C (PTA-126140). In certain embodiments, the Prevotella histicola strain is Prevotella Strain C (PTA-126140).

In some embodiments, the hemoglobin-dependent bacteria are a strain of Prevotella bacteria comprising one or more proteins listed in Table 1. In some embodiments, the hemoglobin-dependent bacteria are from a strain of Prevotella substantially free of one or more of the proteins listed in Table 2.

In some embodiments, the hemoglobin-dependent bacteria are of the genus Fournierella. In some embodiments, the hemoglobin-dependent bacteria are Fournierella Strain A.

In some embodiments, the hemoglobin-dependent Fournierella strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Fournierella Strain B (ATCC Deposit Number PTA-126696, deposited on Mar. 5, 2020). In certain embodiments, the Fournierella strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Fournierella Strain B (PTA-126696). In certain embodiments, the Fournierella strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Fournierella Strain B (PTA-126696). In certain embodiments, the Fournierella strain is Fournierella Strain B (PTA-126696).

In some embodiments, the hemoglobin-dependent bacteria are of the genus Parabacteroides. In some embodiments, the hemoglobin-dependent bacteria are Parabacteroides Strain A. In some embodiments, the hemoglobin-dependent bacteria are Parabacteroides Strain B.

In some embodiments, the hemoglobin-dependent bacteria are of the genus Bacteroides. In some embodiments, the hemoglobin-dependent bacteria are Bacteroides Strain A.

In some embodiments, the hemoglobin-dependent bacteria are of the genus Allistipes. In some embodiments, the hemoglobin-dependent bacteria are Allistipes Strain A.

In some embodiments, the growth medium comprises at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a hemoglobin substitute provided herein. In some embodiments, the growth medium comprises at least 1 g/L and no more than 2 g/L of a hemoglobin substitute provided herein. In some embodiments, the growth medium comprises about 1 g/L of a hemoglobin substitute provided herein. In some embodiments, the growth medium comprises about 2 g/L of a hemoglobin substitute provided herein. In some embodiments, the growth medium comprises yeast extract, soy peptone A2SC 19649, Soy peptone E110 19885, dipotassium phosphate, monopotassium phosphate, L-cysteine-HCl, ammonium chloride, glucidex 21 D, and/or glucose. In some embodiments, the growth media comprises about 5 g/L glucose, about 10 g/L yeast extract 19512, about 10 g/L soy peptone A2 SC 19649, about 10 g/L soypeptone E110 19885, about 2.5 g/L dipotassium phosphate K2HPO4, and about 0.5 g/L L-cysteine-HCl. In some embodiments, the growth medium is at a pH of 5.5 to 7.5. In certain embodiments, the growth medium is at a pH of about 6.5.

In some embodiments of the methods and compositions provided herein, the growth medium does not comprise hemoglobin or a derivative thereof. In certain embodiments, the growth medium does not comprise animal products.

In some embodiments of the methods and compositions provided herein, the hemoglobin-dependent bacteria grow at an increased rate in the growth medium comprising a hemoglobin substitute provided herein (e.g., spirulina or a component thereof) compared to the rate at which the hemoglobin-dependent bacteria grow in the same growth medium but without the hemoglobin substitute (e.g., in the absence of hemoglobin). In some embodiments, the rate at which the hemoglobin-dependent bacteria grow in the growth medium comprising the a hemoglobin substitute provided herein (e.g., spirulina or a component thereof) is at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400% higher than the rate at which the hemoglobin-dependent bacteria grow in the same growth medium but without the hemoglobin substitute. In some embodiments, the growth rate is increased by 200% to 400%.

In certain embodiments of the methods and compositions provided herein the hemoglobin-dependent bacteria grow to a higher cell density in the growth medium comprising a hemoglobin substitute provided herein (e.g., spirulina or a component thereof), compared to the cell density to which the hemoglobin-dependent bacteria grow in the same growth medium but without the hemoglobin substitute (e.g., in the absence of hemoglobin). In some embodiments, the hemoglobin-dependent bacteria grow to a cell density in the growth medium comprising a hemoglobin substitute provided herein (e.g., spirulina or a component thereof) that is at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400% higher than the cell density to which the hemoglobin-dependent bacteria grow in the same growth medium but without the hemoglobin substitute. In some embodiments, the bacterial cell density is 200% to 400% higher.

In certain aspects, provided herein is a bacterial composition (e.g., a pharmaceutical composition) comprising hemoglobin-dependent bacteria disclosed herein and a hemoglobin substitute disclosed herein.

### BRIEF DESCRIPTION OF THE FIGURES

FIG. 1 shows that vitamin B12 and/or FeCl2 cannot substitute for hemoglobin to facilitate growth of hemoglobin-dependent bacteria. FIG. 1 growth curves of the hemoglobin-dependent bacteria Prevotella histicola cultured in the growth media supplemented with 0.02 g/L or 0.2 g/L vitamin B12, FeCl2, or a combination of both, compared to the growth media without any supplement.

FIG. 2 shows that spirulina but not chlorophyllin supports growth of hemoglobin-dependent bacteria in the absence of hemoglobin. FIG. 2 shows growth curves of Prevotella histicola cultured in the growth media supplemented with 0.02 g/L or 0.2 g/L spirulina or chlorophyllin, compared to the growth media without any supplement.

FIG. 3 shows that spirulina dissolved in water performs better than the spirulina dissolved in 0.01 M NaOH. FIG. 3 shows growth curves of Prevotella histicola cultured in growth media supplemented with 0.02 g/L or 0.2 g/L spirulina dissolved in water or 0.01 M NaOH and in the absence of hemoglobin.

FIG. 4 shows that spirulina and soluble components thereof can substitute for hemoglobin to support growth of hemoglobin-dependent bacteria. FIG. 4 shows the growth curves of Prevotella histicola cultured in growth media supplemented with 0.2 g/L, or 2 g/L of spirulina (filtered or unfiltered) or 0.05 g/L or 0.1 g/L chlorphyllin, compared to the growth media supplemented with hemoglobin or a negative control.

FIG. 5 shows that hemoglobin-dependent bacteria cultured with spirulina (in the absence of hemoglobin) are functionally equivalent to those cultured with hemoglobin. A scatter plot shows the efficacy of Prevotella histicola grown in different culture media in a mouse model for delayed-type hypersensitivity (DTH). Each cohort of mice (5 mice per cohort) were administered with vehicle; 1 mg/kg dexamethasone; 1×10⁹ CFU Prevotella histicola biomass cultured in BM1 media (no B12) comprising 1 g/L spirulina (V3); 1×10⁹ CFU Prevotella histicola biomass cultured in BM1 media comprising 1 g/L spirulina (V4); 1×10⁹ CFU Prevotella histicola biomass cultured in SPYG1 media comprising 1 g/L spirulina (V1); or 10 mg powder of Prevotella histicola cultured in growth media comprising hemoglobin. The bar over the scatter plot represents medians and standard deviations. The asterisks (*** and ****) indicate that the values are statistically significant when compared to control.

FIG. 6 shows that spirulina can substitute for hemoglobin to support growth of hemoglobin-dependent bacteria. FIG. 6 shows the growth curve of Fournierella Strain A cultured in SPY growth media (comprising 5 g/L of N-acetyl-glucosamine (NAG)) supplemented with 1 g/L spirulina compared to the growth media supplemented with 0.02 g/L of hemoglobin, FeCl2, or a negative control.

FIG. 7 shows that spirulina can substitute for hemoglobin to support growth of hemoglobin-dependent bacteria. FIG. 7 shows the growth curve of Fournierella Strain B (PTA-126696) cultured in SPY growth media (comprising 5 g/L of N-acetyl-glucosamine (NAG)) supplemented with 1 g/L spirulina compared to the growth media supplemented with 0.02 g/L of hemoglobin, FeCl2, or a negative control. NAG refers to N-acetyl-glucosamine.

FIG. 8 shows that spirulina can substitute for hemoglobin to support growth of hemoglobin-dependent bacteria. FIG. 8 shows the growth curve of Parabacteroides Strain A cultured in SPYG5 growth media supplemented with 1 g/L spirulina compared to the growth media supplemented with 0.02 g/L of hemoglobin, FeCl2, or a negative control. SPYG5 refers to the SPY growth media (Table 6) supplemented with 5 g/L glucose.

FIG. 9 shows that Parabacteroides strain B growth is partially restored by addition of spirulina in comparison to hemoglobin. No growth is observed without addition of hemoglobin or spirulina.

FIG. 10 shows that Faecalibacterium Strain A growth in the presence of spirulina compared to growth of the same strain in hemoglobin containing media or media lacking spirulina or hemoglobin.

FIG. 11 shows that Bacteroides Strain A growth is supported by the presence of spirulina in its growth medium. Without addition of spirulina to the medium the strain does not grow.

FIG. 12 shows that Alistipes Strain A growth in medium containing spirulina compared to medium containing hemoglobin or medium without spirulina or hemoglobin.

### DETAILED DESCRIPTION

In certain aspects, provided herein are methods and compositions that allow for the culturing of hemoglobin-dependent bacteria in the absence of hemoglobin, hemoglobin derivatives, and/or, in certain embodiments, any animal products. Specifically, disclosed herein are hemoglobin substitutes that can be substituted for hemoglobin in culture media to facilitate the growth of hemoglobin-dependent bacteria. In certain embodiments, the hemoglobin substitute can be a cyanobacteria (e.g., cyanobacteria of the genus Arthrospira, such as Arthrospira platensis and/or Arthrospira maxima), a biomass of cyanobacteria (e.g., spirulina), a component of cyanobacteria (e.g., a component of cyanobacteria of the genus Arthrospira, such as Arthrospira platensis and/or Arthrospira maxima and/or a component of spirulina), a green algae, and or a component of green algae.

Thus, in certain aspects, provided herein are methods and compositions for culturing hemoglobin-dependent bacteria in growth media that includes a hemoglobin substitute provided herein. In some aspects, provided herein are compositions (e.g., growth media) comprising a hemoglobin substitute provided herein that are useful for culturing hemoglobin-dependent bacteria in conditions free of hemoglobin or derivatives thereof, as well as methods of making and/or using such compositions.

### Definitions

As used herein, “anaerobic conditions” are conditions with reduced levels of oxygen compared to normal atmospheric conditions. For example, in some embodiments anaerobic conditions are conditions wherein the oxygen levels are partial pressure of oxygen (pO₂) no more than 8%. In some instances, anaerobic conditions are conditions wherein the pO₂ is no more than 2%. In some instances, anaerobic conditions are conditions wherein the pO₂ is no more than 0.5%. In certain embodiments, anaerobic conditions may be achieved by purging a bioreactor and/or a culture flask with a gas other than oxygen such as, for example, nitrogen and/or carbon dioxide (CO₂).

As used herein, “derivatives” of hemoglobin include compounds that are derived from hemoglobin that can facilitate growth of hemoglobin-dependent bacteria. Examples of derivatives of hemoglobin include hemin and protoporphyrin.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

### Hemoglobin-Dependent Bacteria

In some aspects, provided herein are methods and compositions for culturing hemoglobin-dependent bacteria. As used herein, “hemoglobin dependent bacteria” refers to bacteria for which growth rate is slowed and/or maximum cell density is reduced when cultured in growth media lacking hemoglobin, a hemoglobin derivative or spirulina when compared to the same growth media containing hemoglobin, a hemoglobin derivative or spirulina. In some embodiments, the hemoglobin-dependent bacteria are selected from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

In some embodiments, the hemoglobin-dependent bacteria are of the genus Fournierella. In some embodiments, the hemoglobin-dependent bacteria are Fournierella Strain A.

In some embodiments, the hemoglobin-dependent Fournierella strain is Fournierella Strain B (ATCC Deposit Number PTA-126696). In some embodiments, the hemoglobin-dependent Fournierella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Fournierella Strain B (PTA-126696).

In some embodiments, the hemoglobin-dependent bacteria are of the genus Parabacteroides. In some embodiments, the hemoglobin-dependent bacteria are Parabacteroides Strain A. In some embodiments, the hemoglobin-dependent bacteria are Parabacteroides Strain B.

In some embodiments, the hemoglobin-dependent bacteria are of the genus Faecalibacterium. In some embodiments, the hemoglobin-dependent bacteria are Faecalibacterium Strain A.

In some embodiments, the hemoglobin-dependent bacteria are of the genus Bacteroides. In some embodiments, the hemoglobin-dependent bacteria are Bacteroides Strain A.

In some embodiments, the hemoglobin-dependent bacteria are of the genus Allistipes. In some embodiments, the hemoglobin-dependent bacteria are Allistipes Strain A.

In some embodiments, the hemoglobin-dependent bacteria are of the genus Prevotella. In some embodiments, the hemoglobin-dependent bacteria are of the species Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella melanogenica, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptoniphilus lacrimalis, Rarimicrobium hominis, Shuttleworthia satelles, or Turicibacter sanguinis.

In some embodiments, the hemoglobin-dependent Prevotella strain is Prevotella Strain B 50329 (NRRL accession number B 50329). In some embodiments, the hemoglobin-dependent Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the hemoglobin-dependent Prevotella strain is Prevotella Strain C (ATCC Deposit Number PTA-126140). In some embodiments, the hemoglobin-dependent Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain C (PTA-126140).

In some embodiments, the hemoglobin-dependent Prevotella strain is a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) genes encoding proteins listed in Table 1. In some embodiments, the hemoglobin-dependent Prevotella strain comprises all of the proteins listed in Table 1 and/or all of the genes encoding the proteins listed in Table 1.

TABLE 1
Exemplary Prevotella proteins

| Seq. | | | |
|---|---|---|---|
| ID. | | Uniprot | Amino Acid |
| No. | Name | ID | Sequence |
| 1 | Cluster: | G6ADE1 | MNLKTFTKTVLCFAL |
| | Uncharacterized | | FAVSAITAKAADHLA |
| | protein | | IVGEAVWGGWDLVKA |
| | | | TAMVKSPNNPDVFMA |
| | | | TVHLNAGKGFKFLTE |
| | | | REWGKLEYRSGASDV |
| | | | VLKSGIRYKLYASIG |
| | | | ASEDGKFKVSESANY |
| | | | EIICDLARKTVEVKK |
| | | | VAYQAKEIRYAALWM |
| | | | IGDATAGDWDYNNGV |
| | | | LLSQDSGNPTCYTAT |
| | | | VELKEGEFKFTTNKQ |
| | | | WGYDHSVYIFRDVND |
| | | | QNKIVFGGEDNKWRI |
| | | | TEDGMYNVTVDVPTK |
| | | | TISIKQIDDPAGHKP |
| | | | QFGNDVILVGDATIA |
| | | | GWNLDNAIYLEHTGQ |
| | | | AGRVFKTTTYLEAGK |
| | | | GFKFLSMLSYDDIDY |
| | | | RPANNTVLNPGVPGT |
| | | | FVPSLPSSTDTKFSV |
| | | | ERSGNYDIVCNMNNR |
| | | | TVVVTLSENQVLVNY |
| | | | PALWLIGSATSAGWN |
| | | | PGKAVELKRSEADPA |
| | | | VYTARVQLKKGEFKI |
| | | | LTSKNVGFDQPTYYR |
| | | | DSTNEHRIVFGVDGD |
| | | | EVAKKDCKWTLSENA |
| | | | EGTYDVTVDIEAMTI |
| | | | FCDKVNMDEPSVEST |
| | | | DKELILIGDATYSAW |
| | | | DLPKSIVMTPVGPTT |
| | | | FKAVTHLEAGKEFKF |
| | | | LTELAWKRYEYRAES |
| | | | LRKELQEGSMSMLVP |
| | | | YRYTNDKDDKDHDFK |
| | | | FVVKESGNYEIVCDL |
| | | | YIPALIIRKVRYQDT |
| | | | PVTYSSLWIVGSATP |
| | | | GGWTIERGIKMTQDE |
| | | | NYPTKFTAKANLVPG |
| | | | ELKFATNKFADFTQD |
| | | | FFFRGKDDYTAVLGG |
| | | | NDNKWNITEAGTYSV |
| | | | TIDVASKRVTITKPA |
| | | | RNAPTGISTVDSSDE |
| | | | APAEYFTLNGIKVTT |
| | | | PSSGIYIKRQGGRTT |
| | | | KVVMK |
| 2 | Nicotinamide_ | P24520 | MDTYQILDIIGCIVG |
| | riboside_ | | LIYIYQEYKASIWLW |
| | transporter_ | | MTGIIMPVIYMFVYY |
| | PnuC | | EAGLYADFGMQIYYT |
| | | | LAAIYGYLYWKLGKK |
| | | | KGTEDKEIPITHFPR |
| | | | RYIIPAIIVFFVLWI |
| | | | ALYYILICFTNSTVP |
| | | | VLDSFGNALSFIGLW |
| | | | ALAKKYLEQWWIWIV |
| | | | VDAELSALYIYKGIP |
| | | | FTAMLYALYTVIAVA |
| | | | GYFKWRRYIKQQK |
| 3 | Pectate_ | Q8GCB2 | MRVRLYKNILLFLFL |
| | trisaccharide- | | WVNTLACVSADTSRT |
| | lyase | | VESQPIENGLIITES |
| | | | KGWLETIYAKWKPVA |
| | | | EADGYYVYVKGGQYA |
| | | | DYSKVDSELIRVYNG |
| | | | YVRVDIPGLKAGTYS |
| | | | LKIVAVKGGKETQSS |
| | | | EVTGLKVLNYVREGF |
| | | | AHKNYSGVGAYNDDG |
| | | | TLKSGAVVIYVNKDN |
| | | | AKTVSAHLGKTTFIG |
| | | | LQAILNAYQKGNITT |
| | | | PLSVRILGLLRNGDT |
| | | | DTFGSSTEGIQIKGK |
| | | | QADSEMNITIEGIGE |
| | | | DASIYGFGFLVRNAK |
| | | | SVEFRNLGIMRAMDD |
| | | | GVSLDTNNSNIWIHH |
| | | | MDLFYGKASGGDHIK |
| | | | GDGSIDVKTDSKYVT |
| | | | IDNCHFWDTGKTSMC |
| | | | GMKKETGPNYITYHH |
| | | | NWFDHSDSRHARVRT |
| | | | MSVHLWNNYYDGCAK |
| | | | YGIGATMGCSVFSEN |
| | | | NYFRATKNPILISKQ |
| | | | GSDAKGTGKFSGEPG |
| | | | GMVKEYGSLFTEKGA |
| | | | ESTYTPISYADNNSS |
| | | | FDFYHAISRNEKVPA |
| | | | SVKTLNGGNIYNNFD |
| | | | TDAALMYSYTPDATA |
| | | | LVPSQVTGFYGAGRL |
| | | | NHGSLQFKFNNAVED |
| | | | TNSTPIPALEALIDA |
| | | | YSGK |
| 4 | Glycosyl | Q9AET5 | MKYNIAYCIEGFYNH |
| | transferase_ | | GGMERILSVCANLLS |
| | Gtf1 | | DIYSITIIVANQRGR |
| | | | EHAYNLAQNVNVVDL |
| | | | GVSCKNYKEEYKKSL |
| | | | TRYLQDHQFSVVISL |
| | | | AGLELFFLPQIKDGS |
| | | | KKVMWFHFAFDVSKM |
| | | | FLSERFHGWKLNLLY |
| | | | YIHTIRRIYFAKKFD |
| | | | TIVVLSKSDCDSWSR |
| | | | FCNNVKYIYNPITID |
| | | | RKVISNLSEESVIAV |
| | | | GRLGWQKGFDFLIDS |
| | | | WVLVDDKHPDWFILD |
| | | | IFGEGPDRLELQFIQ |
| | | | IDRKGLHDKVRLCGV |
| | | | TKQIEEEYGKHSIYV |
| | | | MSSRAEGFPLALLEA |
| | | | SSCGLPMISFNCHQG |
| | | | PNEIIQEGENGFLVD |
| | | | KVGDIYTLSDRICKL |
| | | | IEDNNLRNMMGKKAL |
| | | | DSSFRFEGEVIKKDW |
| | | | ISLLKQLI |
| 5 | Cluster: | A0A096B75 | MKRLFFMFLFLGTIT |
| | Protein | 9 | MNSLAQEEKPIKYET |
| | TonB | | KNFSLPDKMPLYPGG |
| | | | DGALRAFLSLNLFTY |
| | | | PEKAQAFGVEGRSLM |
| | | | KFCVSSDGSIKDISA |
| | | | VDCKITNYNRTEFNK |
| | | | LPLSKQESLKKECAK |
| | | | AFAKEAARVIRLMPK |
| | | | WEPAELNGKKMNVYY |
| | | | SLPFTFKLR |
| 6 | Cluster: | G6AEN6 | MNYPLFIARKIYNGG |
| | Uncharacterized | | DRTRKVSKPAIRIAT |
| | protein | | IGVAIGLAVMIISVG |
| | | | VVLGFKHTIRNKVVG |
| | | | FGSDITVANFLTLQS |
| | | | SEQYPIQITDSLVKS |
| | | | LQITPGIKHVQRYDY |
| | | | TQGILKTDNDFLGVL |
| | | | LKGVGPDFDSTFIHE |
| | | | NMVEGSLPHFHDNES |
| | | | QQKIVISKTIADKLN |
| | | | LKVGQRIFAYFINKQ |
| | | | GVRTRKFTITGIYAT |
| | | | NMKQFDSQICFTDIY |
| | | | TTNKLNGWEPDQYSG |
| | | | AELQVDNFSQLTPIS |
| | | | MRVLNKVKNTVDHYG |
| | | | GTYSSENIIEQNPQI |
| | | | FSWLDLMDMNVWIIL |
| | | | ALMISVAGVTMISGL |
| | | | LIIILERTQMIGILK |
| | | | ALGSRNRQIRHIFLW |
| | | | FATFIIGKGLLWGNI |
| | | | IGLGCILFQSWTGLV |
| | | | KLDPQTYYVNTVPVE |
| | | | INIPLIIALNMVTML |
| | | | VCLVILIAPSYLISH |
| | | | IHPAKSMHYE |
| 7 | Bifunctional_ | P9WHG9 | MEDKFIYTDKERKLS |
| | (p)ppGpp_ | | YQILDELKDTLDKSF |
| | synthase/ | | LENDLPMLQVQLKDS |
| | hydrolase_ | | VAKNTIHRNVFGLNP |
| | RelA | | ILCSLQTAAIAVKDI |
| | | | GLKRDSVIAILLHQS |
| | | | VQDGYITLEDIDNRF |
| | | | GKSVAKIIHGLIRIQ |
| | | | TLYQKNPIIESENFR |
| | | | NLLLSFAEDMRVILI |
| | | | MIADRVNLMRQIRDA |
| | | | EDKEAQHKVAEEASY |
| | | | LYAPLAHKLGLYQLK |
| | | | RELEDLSLKYLEHDA |
| | | | YYLI |
| | | | KDKLNATKASRDAYI |
| | | | NQFIAPVRERLTAGG |
| | | | LRFHIKGRTKSIHSI |
| | | | WQKMKKQKCGFEGIY |
| | | | DLFAIRIILDAPLEK |
| | | | EKIQCWQAYSIVTDM |
| | | | YQPNPKRLRDWLSVP |
| | | | KSNGYECLHITVLGP |
| | | | EKKWVEVQIRTERMD |
| | | | EIAEHGLAAHWRYKG |
| | | | IKEEGGLDDWLASIR |
| | | | AALEAGDNLEVMDQF |
| | | | KSDLYEKEIYVFTPK |
| | | | GDLLKFPKGATILDF |
| | | | AYHIHSKVGNQCVGG |
| | | | KINAKNVSLRTELHS |
| | | | GDTVEILTSATQKPK |
| | | | AEWLKIVKSSRAKAK |
| | | | IRLALKETQIKDGLY |
| | | | AKELLERRFKNKKIE |
| | | | IEESTMGHLLRKLGF |
| | | | KEVSEFYKQVADEKL |
| | | | DPNYIIEEYQKVYNH |
| | | | DHNLNQPKETESAEN |
| | | | FEFENPTNEFLKKND |
| | | | DVLVIDKNLKGLDFS |
| | | | LAKCCHPIYGDPVFG |
| | | | FVTVNGGIKIHRTDC |
| | | | PNAPEMRKRFGYRIV |
| | | | KARWSGKGSSQYAIT |
| | | | LRVIGNDDIGIVSNI |
| | | | TNVISKDEKIVMRSI |
| | | | NIDSHDGLFSGNLVV |
| | | | LLDDNSKLNMLIKKL |
| | | | RTVKGVKQVTRI |
| 8 | Vitamin_B12_ | P06609 | MKRRIFLFVALSVSI |
| | import_system_ | | VILFGLNLIIGSVHI |
| | permease_ | | PLSDILTILSGSFTG |
| | protein_BtuC | | KESWRFIIWDSRLPQ |
| | | | ALTAMLCGSSLAVCG |
| | | | LMLQTAFRNPLAGPD |
| | | | VFGISSGASLGVALV |
| | | | MLLLGGTVETSMFTA |
| | | | SGFLAILIVAFAGAI |
| | | | LVTAFILFLSSVVRN |
| | | | SVLLLIVGIMVGYVA |
| | | | SSAVTLLNFFSSEDG |
| | | | VKGYIVWGMGNFGGV |
| | | | SMSHIPLFAFLCLAG |
| | | | IIASFLLVKPLNILL |
| | | | LGPQYAESLGISIRR |
| | | | IRNILLVVVGILTAV |
| | | | TTAFCGPISFIGLAA |
| | | | PHVARLLFRTENHQK |
| | | | LLPGTLLVGTVVALL |
| | | | CNLICFLPRESGMIP |
| | | | LNAVTPLIGAPIIIY |
| | | | VIMKRH |
| 9 | NADH- | P33599 | MKLENKEFGFDSFAT |
| | quinone_ | | EMARLKNEKHFDYLV |
| | oxidored | | TVVGEDFGTEEGLGC |
| | uctase_ | | IYILENTSTHERCSV |
| | subunit_C/D | | KQLAKKVGEEFVIPS |
| | | | VIKLWADADLLEREV |
| | | | YDFYGIKFLGHPDMR |
| | | | RLFLRNDFKGYPLRK |
| | | | DYDMDPAKNMYTTED |
| | | | DVELDTTTEWNLDKN |
| | | | GELVGTQHALFTDDN |
| | | | FVVNIGPQHPSTHGV |
| | | | LRLQTVLDGETVTNI |
| | | | YPHLGYIHRGIEKLC |
| | | | EQFTYPQTLALTDRM |
| | | | NYLSAMMNRFLALVG |
| | | | VIEEGMGIELSERIL |
| | | | YIRTIMDELQRIDNH |
| | | | LLYTACCAQDLGALT |
| | | | AFLYGMRDREHVLNV |
| | | | MEETTGGRLIQNYYR |
| | | | IGGLQADIDPNFVSN |
| | | | VKELCKYLRPMIQEY |
| | | | VDVFGDNVITHQRFE |
| | | | GVGVMDEKDCISYGV |
| | | | TGPAGRASGWKNDVR |
| | | | KYHPYAMYDKVNFEE |
| | | | ITLTNGDSMDRYFCH |
| | | | IKEIYQSLNIIEQLI |
| | | | DNIPEGEFYIKQKPI |
| | | | IKVPEGQWYFSVEGA |
| | | | SGEFGAYLDSRGDKT |
| | | | AYRLKFRPMGLTLVG |
| | | | AMDKMLRGQKIADLV |
| | | | TTGAALDFVIPDIDR |
| 10 | FKBP- | P45523 | MRTSTQSKDMGKKQE |
| | type_ | | YKLRNEEFLHNISKK |
| | peptidyl- | | DSIKTLPHGIFYEII |
| | prolyl_ | | KEGSGEGTVQPRSIV |
| | cis- | | ICNYRGSLISGQVFD |
| | trans_ | | DSWQKPTPEAFRLNE |
| | isomerase | | LITGLQIALCAMHKG |
| | | | DSWRIYIPYQEGYGS |
| | | | KRNADIPAFSTLIFD |
| | | | IELINIA |
| 11 | Putative_ | P9WKJ3 | MADNKIAKESVKREV |
| | acetolactate_ | | IAGERLYTLLVYSEN |
| | synthase_ | | VAGVLNQIAAVFTRR |
| | small_ | | QVNIESLNVSASSIE |
| | subunit | | GIHKYTITAWSDAAT |
| | | | IEKITKQVEKKIDVI |
| | | | KADYYEDSDLFIHEV |
| | | | GLYKIATPILLENAE |
| | | | VSRAIRKRNARMMEV |
| | | | NPTYSTVLLAGMTDE |
| | | | VTALYHDLKNFDCLL |
| | | | QYSRSGRVAVTRGFS |
| | | | EPVSDFLKSEEESSV |
| | | | L |
| 12 | Serine/ | P0AGE4 | MKKKVKIGLLPRVII |
| | threonine_ | | AILLGIFFGYFMPTP |
| | transporter_ | | LARVFLTFNGIFSQF |
| | SstT | | LGFMIPLIIIGLVTP |
| | | | AIADIGKGAGKLLLV |
| | | | TVIIAYVDTVVAGGL |
| | | | AYGTGLCLFPSMIAS |
| | | | TGGAMPHIDKATELA |
| | | | PYFSINIPAMADVMS |
| | | | GLVFSFMLGLGIAYG |
| | | | GLTATKNIFNEFKYV |
| | | | IEKVIAKAIIPLLPL |
| | | | YIFGVFLNMAHNGQA |
| | | | QQILLVFSQIIIVIL |
| | | | VLHVFILVYQFCIAG |
| | | | AIIRRNPFRLLWNMM |
| | | | PAYLTALGTSSSAAT |
| | | | IPVTLEQTMKNGVGK |
| | | | EIAGFVVPLCATIHL |
| | | | SGSAMKITACALTIC |
| | | | LLVGLPHDPALFIYF |
| | | | ILMLSIIMVAAPGVP |
| | | | GGAIMAALAPLASIL |
| | | | GFNSEAQALMIALYI |
| | | | AMDSFGTACNVTGDG |
| | | | AIALVVNKMFGKKER |
| 13 | Cluster: | G6AJ07 | MKKLLLLVCAAVMSL |
| | Uncharacterized | | SASAQAGDKALGAQL |
| | protein | | VFGSETNSLGFGVKG |
| | | | QYYFTDHIRGEGSFD |
| | | | YFLKNKGISMWDINA |
| | | | NVHYLFDVADKFKVY |
| | | | PLAGLGYTNWSYKYE |
| | | | YAGAPVVEGSDGRLA |
| | | | VNLGGGVEYELTKNL |
| | | | NVNAEAKYQIISNYN |
| | | | QLVLGVGVAYKF |
| 14 | Heterocyst_ | P22638 | MHFYCTKSSLDTMSE |
| | differentiation_ | | RYVKRMIAKLASQGK |
| | ATP- | | TVISIAHRFSTIMDA |
| | binding_ | | KHIILLAKGKVVAEG |
| | protein | | THQELLKTSEDYRKL |
| | | | WSDQNDEID |
| 15 | UDP-2,3- | Q9I2V0 | MKNVYFLSDAHLGSL |
| | diacylglucosamine_ | | AIAHRRTQERRLVRF |
| | hydrolase | | LDSIKHKASAVYLLG |
| | | | DMFDFWDEYKYVVPK |
| | | | GFTRFLGKVSELTDM |
| | | | GVEVHFFTGNHDLWT |
| | | | YGYLEEECGVILHRK |
| | | | PVTMEIYGKVFYLAH |
| | | | GDGLGDPDPMFQFLR |
| | | | KVFHNRVCQRLLNFF |
| | | | HPWWGMQLGLNWAKK |
| | | | SRLKRADGKEMPYLG |
| | | | EDKEYLVRYTKDYMR |
| | | | SHKDIDYYIYGHRHI |
| | | | ELDLTLSGKVRMLIL |
| | | | GDWIWQFTYAVFDGE |
| | | | HMFLEEYIEGESKP |
| 16 | Anaerobic_ | P0A9C0 | MNSKQNDNYDVIIIG |
| | glycerol-3- | | GGITGAGTARDCALR |
| | phosphate_ | | GLKVLLVEKFDFTNG |
| | dehydrogenase | | ATGRNHGLLHSGARY |
| | | | AVTDPESATECIKEN |
| | | | MVLRRIAKHCIEETD |
| | | | GLFITLPEDDINYQK |
| | | | TFVEACARAGISANI |
| | | | ISPEEALRLDPSVNP |
| | | | DLLGAVRVPDASVDP |
| | | | FHLTTANVLDARQHG |
| | | | ADVLTYHEVVAILTS |
| | | | NGRVEGVRLRNNHTG |
| | | | EEIEKHAVLVINAAG |
| | | | IWGHDIAKMADIKIN |
| | | | MFPAKGTLLVFGHRV |
| | | | NKMVINRCRKPANAD |
| | | | ILVPDDAVCVIGTTS |
| | | | DRVPYDTVDNLKITS |
| | | | EEVDTLIREGEKLAP |
| | | | SLATTRILRAYAGVR |
| | | | PLVAADNDPTGRSIS |
| | | | RGIVCLDHEKRDGLT |
| | | | GMITITGGKMMTYRL |
| | | | MAEQATDLACKKLGI |
| | | | NKTCETATTPLPGTA |
| | | | GKDSDNPHHTYSTAH |
| | | | KAAKGRQGNRVKEID |
| | | | ERTEDDRALICECEE |
| | | | VSVGEAKYAIEELHV |
| | | | HDLLNLRRRTRVGMG |
| | | | TCQGELCACRAAGVM |
| | | | CENGVKVDKAMTDLT |
| | | | KFINERWKGMRPVAW |
| | | | GSTLDEAQLTTHYQG |
| | | | LCGLGI |
| 17 | Anaerobic_ | PI3033 | MRYDTIIIGGGLSGL |
| | glycerol-3- | | TAGITLAKAGQKVCI |
| | phosphate_ | | VSAGQSSLHFHSGSF |
| | dehydrogenase | | DLLGYDADGEVVTHP |
| | | | LQAIADLKAEHPYSK |
| | | | IGISNIEHLASQAKT |
| | | | LLCEAGISVMGNYEQ |
| | | | NHYRVTPLGTLKPAW |
| | | | LTTEGYAMIDDPEIL |
| | | | PWKKVELLNIQGFMD |
| | | | FPTQFIAENLRMMGV |
| | | | ECQIKTFTTDELSTA |
| | | | RQSPTEMRATNIAKV |
| | | | LANKDALSKVSERIN |
| | | | AISGDPDALLLPAVL |
| | | | GFSNAESLDEMKQWI |
| | | | KKPVQYIATLPPSVS |
| | | | GVRTTILLKRLFAQA |
| | | | GGTLLIGDSATTGQF |
| | | | SGNHLVSITTDHLPD |
| | | | EKLYADHFILASGSF |
| | | | MSHGIRSNYAGVYEP |
| | | | VFKLDVDAAEKRDDW |
| | | | SVTNAFEAQPYMEFG |
| | | | VHTDKDFHATKDGKN |
| | | | IENLYAIGSVLSGHN |
| | | | SIKHADGTGVSLLTA |
| | | | LYVAKKITGKG |
| 18 | Anaerobic_ | P0A996 | MAEGIQLKNISGNNL |
| | glycerol-3- | | EQCLKCSICTAYCPV |
| | phosphate_ | | SAVEPKYPGPKQSGP |
| | dehydrogenase | | DQERYRLKDSKFFDE |
| | | | ALKMCLNCKRCEVAC |
| | | | PSGVRIADIIQASRI |
| | | | TYSTHRPIPRDIMLA |
| | | | NTDFVGTMANMVAPI |
| | | | VNATLGLKPVKAVLH |
| | | | GVMGIDKHRTFPAYS |
| | | | SQKFETWYKRMAAKK |
| | | | QDSYSKHVSYFHGCY |
| | | | VNYNFPQLGKDLVKI |
| | | | MNAVGYGVHLLEKEK |
| | | | CCGVALIANGLSGQA |
| | | | RRQGKVNIRSIRKAA |
| | | | EQNRIVLTTSSTCTF |
| | | | TMRDEYEHLLDIKTD |
| | | | DVRENITLATRFLYR |
| | | | LIEKGDIKLAFRKDF |
| | | | KMRTAYHSACHMEKM |
| | | | GWIIYSTELLKMIPG |
| | | | LELIMLDSQCCGIAG |
| | | | TYGFKKENYQRSQEI |
| | | | GEGLFKQIKELNPDC |
| | | | VSTDCETCKWQIEMS |
| | | | TGYEVKNPISILADA |
| | | | LDVEETIKLNQ |
| 19 | Glycerol_ | P18156 | MMIKNIVLSIPISLI |
| | uptake_ | | IYLNHLIMEYSMTTQ |
| | facilitator_ | | FLMELIGTLILVLFG |
| | protein | | DGVCACVTLNKSKGQ |
| | | | KAGWVVITIAWGLAV |
| | | | CMGVLVAGPYTGAHL |
| | | | NPAVSIGLAVAGMFP |
| | | | WSSVPYYIVAQMIGG |
| | | | FLGGLLVWFFYKDHY |
| | | | DATDDEAAKLGTFCT |
| | | | SPAIRNYKMNFLSEV |
| | | | IATLVLVFIIISFSV |
| | | | DGNTGDAEHFKFGLA |
| | | | ALGPIPVTLLIIALG |
| | | | MSLGGTTGYAMNPAR |
| | | | DLSPRLAHAVCMKGD |
| | | | NDWSYSWIPVLGPII |
| | | | GAIIAGFCGAALLLV |
| 20 | Serine/ | Q97PA9 | MSEKIIPSNEPAQAA |
| | threonine- | | SEPIKASYTEYTVIP |
| | protein_ | | SQGYCQFVKCKKGDQ |
| | kinase_ | | PVVLKGLKEAYRERV |
| | StkP | | LLRNALKREFKQCQR |
| | | | LNHPGIVRYQGLVDV |
| | | | EGYGLCIEEEYVDGR |
| | | | TLQAYLKESHTDDEK |
| | | | ITIVNQIADALRYAH |
| | | | QQGVAHRNLKPSNIL |
| | | | ITKQGDHVKLIDFNV |
| | | | LSLDDVKPTADTTRF |
| | | | MAPELKDETMTADGT |
| | | | ADIYSLGTIMKVMGL |
| | | | TLAYSEVIKRCCAFK |
| | | | RSDRYSDIDEFLADF |
| | | | NHDGSSFSMPKIGKG |
| | | | TVVIGFIAVVVIALA |
| | | | ALAYNYGGALVDQVG |
| | | | KIDVTSIFKSDAETA |
| | | | PEDSAMVKSVEQNNN |
| | | | DSVADEAPATGKLAF |
| | | | MNTMKPALYKDLDRL |
| | | | FAKHSDDRAKLNRAI |
| | | | KVYYRGLIQANDTLD |
| | | | NEQRAELDRVFGNYV |
| | | | KQKKAALK |
| 21 | Cluster: | G6AHI1 | MLVAQLFVGVLQAQK |
| | D-alanyl- | | PVQNRRQAVGQSMER |
| | D-alanine | | QGLVNVKAVVPSIKV |
| | dipeptidase | | ALMYARTDNFCHRMA |
| | | | LS |
| 22 | Anaerobic_ | P0ABN5 | MITGLVIIQLLIVLA |
| | C4- | | LIFIGARVGGIGLGI |
| | dicarboxylate_ | | YGMIGVFILVYGFGL |
| | transporter_ | | APGSAPIDVMMIIVA |
| | DcuA | | VITAASALQASGGLE |
| | | | YLVGVAAKFLQKHPD |
| | | | HITYFGPITCWLFCV |
| | | | VAGTAHTSYSLMPII |
| | | | AEIAQTNKIRPERPL |
| | | | SLSVIAASLGITCSP |
| | | | VSAATAALISQDLLG |
| | | | AKGIELGTVLMICIP |
| | | | TAFISILVAAFVENH |
| | | | IGKELEDDPEYKRRV |
| | | | AAGLINPEAACEEVQ |
| | | | KAENEHDPSAKHAVW |
| | | | AFLFGVALVILFGFL |
| | | | PQLRPEGVSMSQTIE |
| | | | MIMMSDAALILLVGK |
| | | | GKVGDAVNGNIFKAG |
| | | | MNAVVAIFGIAWMGN |
| | | | TFYVGNEKILDAALS |
| | | | SMISSTPILFAVALF |
| | | | LLSIMLFSQAATVTT |
| | | | LYPVGIALGINPLLL |
| | | | IAMFPACNGYFFLPN |
| | | | YPTEVAAIDFDRTGT |
| | | | TRVGKYVINHSFQIP |
| | | | GFITTIVSILLGVLM |
| | | | VQFFR |
| 23 | L-asparaginase_2 | P00805 | MRILKITFVTVLALV |
| | | | MSTVVFAQKPKIRII |
| | | | ATGGTIAGVSASATS |
| | | | SAYGAGQVGVQTLID |
| | | | AVPQIKDIADVSGEQ |
| | | | LVNIGSQDMNDEVWL |
| | | | KLAKRINDLLNKEGY |
| | | | DGVLITHGTDTMEET |
| | | | AYFLSLTVHTDKPVV |
| | | | MVGSMRPSTAISADG |
| | | | PANLYNGICTLVDPS |
| | | | SKGHGVMVCMNNELF |
| | | | EAKSVIKTHTTDVST |
| | | | FKGGLYGEMGYVYNG |
| | | | KPYFLHKPVAKQGLT |
| | | | SEFNVDNLTSLPKVG |
| | | | IVYGYANCSPLPIQA |
| | | | FVNAKFDGIVLAGVG |
| | | | DGNFYKDVFDVALKA |
| | | | QNSGIQIVRSSRVPF |
| | | | GPTNLNGEVDDAKYH |
| | | | FVASLNLNPQKARVL |
| | | | LMLALTKTKDWQKIQ |
| | | | QYFNEY |
| 24 | Trehalose_ | P9WQ19 | MALACAMTMSASAQM |
| | synthase/ | | GTNPKWLGDAIFYQI |
| | amylase_ | | YPSSYMDTDGNGIGD |
| | TreS | | LPGITQKLDYIKSLG |
| | | | VNAIWLNPVFESGWF |
| | | | DGGYDVIDFYKIDPR |
| | | | FGTNTDMVNLVKEAH |
| | | | KRGIKVCLDLVAGHT |
| | | | STKCPWFKESANGDR |
| | | | NSRYSDYFIWTDSIS |
| | | | EADKKEIAERHKEAN |
| | | | PASSTHGRYVEMNAK |
| | | | RGKYYEKNFFECQPA |
| | | | LNYGFAKPDPNQPWE |
| | | | QPVTAPGPQAVRREM |
| | | | RNIMAFWFDKGVDGF |
| | | | RVDMASSLVKNDWGK |
| | | | KEVSKLWNEMREWKD |
| | | | KNYPECVLISEWSDP |
| | | | AVAIPAGFNIDFMIH |
| | | | FGIKGYPSLFFDRNT |
| | | | PWGKPWPGQDISKDY |
| | | | KFCYFDKAGKGEVKE |
| | | | FVDNFSEAYNATKNL |
| | | | GYIAIPSANHDYQRP |
| | | | NIGTRNTPEQLKVAM |
| | | | TFFLTMPGVPFIYYG |
| | | | DEIGMKYQMDLPSKE |
| | | | GSNERAGTRTPMQWT |
| | | | SGPTAGFSTCNPSQL |
| | | | YFPVDTEKGKLTVEA |
| | | | QQNDPRSLLNYTREL |
| | | | TRLRHSQPALRGNGE |
| | | | WILVSKESQPYPMVY |
| | | | KRTSGGETVVVAINP |
| | | | SDKKVSANIAHLGKA |
| | | | KSLIMTGKASYKTGK |
| | | | TEDAVELNGVSAAVF |
| | | | KIAE |
| 25 | Ribitol-5- | Q720Y7 | MNIAVIFAGGSGLRM |
| | phosphate_ | | HTKSRPKQFLDLNGK |
| | cytidylyl | | PIIIYTLELFDNHPG |
| | transferase | | IDAIVVACIESWIPF |
| | | | LEKQLRKFEINKVVK |
| | | | IVPGGESGQASIYNG |
| | | | LCAAEAYIKSKNVAS |
| | | | EDTTVLIHDGVRPLI |
| | | | TEETITDNINKVAEV |
| | | | GSCITCIPATETLVV |
| | | | KQHDGSLEIPSRADS |
| | | | LIARAPQSFLLSDIL |
| | | | TAHRRAIDEKKNDFI |
| | | | DSCTMMSHYGYRLGT |
| | | | IIGPMENIKITTPTD |
| | | | FFVLRAMVKVHEDQQ |
| | | | IFGL |
| 26 | UDP-Glc: | B5L3F2 | MTEKKSVSIVLCTYN |
| | alpha-D- | | GTKYLQEQLDSILAQ |
| | GlcNAc- | | TYPLHEIIIQDDGST |
| | diphospho- | | DNTWQILEKYEEKYP |
| | undecaprenol | | LIHIYHNEGTHGVNA |
| | | | NFLSAMHRTTGDFIA |
| | | | IADQDDIWETDKIAN |
| | | | QMTTIGNKLLCSGLT |
| | | | RPFSSDGSFAYFDNR |
| | | | PRNVSIFRMMFLGLP |
| | | | GHTMLFRRELLRMMP |
| | | | PVTHSFFNVSLYDAA |
| | | | LSILAASHDSIAFCN |
| | | | KVLVNFRRHADATTY |
| | | | NDYSRSLPSWQNGLY |
| | | | ELLWGLRHYHQARSI |
| | | | ALPIYRGKLALMEGI |
| | | | TTNYHDFIEAKAIMR |
| | | | LETQKGLWAFLRLQY |
| | | | LLTKNHQRLFQTSGG |
| | | | SFIKMIRAWLYPVMQ |
| | | | LYMYHHALRRCK |
| 27 | UDP-N- | P33038 | MESFIIEGGHRLSGT |
| | acetylglucosamine | | IAPQGAKNEALEVIC |
| | | | ATLLTTEEVIIRNIP |
| | | | NILDVNNLIKLLQDI |
| | | | GVKVKKLGANDFSFQ |
| | | | ADEVKLDYLESIDFV |
| | | | KKCSSLRGSVLMIGP |
| | | | LLGRFGKATIAKPGG |
| | | | DKIGRRRLDTHFLGF |
| | | | KNLGARFVRIEDRDV |
| | | | YEIQADKLVGDYMLL |
| | | | DEASVTGTANIIMSA |
| | | | VMAEGTTTIYNAACE |
| | | | PYIQQLCHLLNAMGA |
| | | | KITGIASNLITIEGV |
| | | | TSLHGAEHRILPDMI |
| | | | EVGSFIGMAAMVGDG |
| | | | VRIKDVSIPNLGLIL |
| | | | DTFRRLGVQIIEDED |
| | | | DLIIPRQDHYVIDSF |
| | | | IDGTIMTISDAPWPG |
| | | | LTPDLISVLLVVATQ |
| | | | AQGSVLFHQKMFESR |
| | | | LFFVDKLIDMGAQII |
| | | | LCDPHRAVWGHDHAK |
| | | | KLRAGRMSSPDIRAG |
| | | | IALLIAALTAEGTSR |
| | | | IDNIAQIDRGYENIE |
| | | | GRLNALGAKVQRVEI |
| | | | C |
| 28 | Sensor_ | P30855 | MERSGNFYKAIRLGY |
| | protein_EvgS | | ILISILIGCMAYNSL |
| | | | YEWQEIEALELGNKK |
| | | | IDELRKEINNINIQM |
| | | | IKFSLLGETILEWND |
| | | | KDIEHYHARRMAMDS |
| | | | MLCRFKATYPAERID |
| | | | SVRHLLEDKERQMCQ |
| | | | IVQILEQQQAINDKI |
| | | | TSQVPVIVQKSVQEQ |
| | | | PKKSKRKGFLGIFGK |
| | | | JKEEAKPTVTTTMHR |
| | | | SFNRNMRTEQQAQSR |
| | | | RLSVHADSLAARNAE |
| | | | LNRQLQGLVVQIDGK |
| | | | VQTDLQKREAEITAM |
| | | | RERSFIQIGGLTGFV |
| | | | ILLLVISYIIIHRNA |
| | | | NRIKRYKQETADLIE |
| | | | RLQQMAKRNEALITS |
| | | | RKKAVHTITHELRTP |
| | | | LTAITGYAGLIQKNF |
| | | | NADKTGMYIRNIQQS |
| | | | SDRMREMLNTLLSFF |
| | | | RLDDGKEQPNFSTCR |
| | | | ISSIAHTLESEFMPI |
| | | | AINKGLALTVTNHTD |
| | | | AVVLTDKERILQIGN |
| | | | NLLSNAIKFTENGAV |
| | | | SLTMGYDNGMLKLIV |
| | | | KDTGSGMTEEEQQRV |
| | | | FGAFERLSNAAAKDG |
| | | | FGLGLSIVQRIVTML |
| | | | GGTIQLKSEKGKGSR |
| | | | FTVEIPMQSAEELPE |
| | | | RINKTQIHHNRTLHD |
| | | | IVAIDNDKVLLLMLK |
| | | | EMYAQEGIHCDTCTN |
| | | | AAELMEMIRRKEYSL |
| | | | LLTDLNMPDINGFEL |
| | | | LELLRTSNVGNSRII |
| | | | PIIVTTASGSCNREE |
| | | | LLERGFSDC |
| | | | LLKPFSISELMEVSD |
| | | | KCAMKGKQNEKPDFS |
| | | | SLLSYGNESVMLDKL |
| | | | IAETEKEMQSVRDGE |
| | | | QRKDFQELDALTHHL |
| | | | RSSWEILRADQPLRE |
| | | | LYKQLHGSAVPDYEA |
| | | | LNNAVTAVLDKGSEI |
| | | | IRLAKEERRKYENG |
| 29 | Phosphate- | Q7A5Q2 | MKRSRFYITVGLILS |
| | binding_ | | LTLLMSACGQKKAKD |
| | protein_ | | GRTDTPTSGTIKFAS |
| | PstS | | DESFSPIVEELLQNY |
| | | | QFRYPQAHLLPIYTD |
| | | | DNTGMKLLLDQKVNL |
| | | | FITSHAMTKGEDAIL |
| | | | RGKGPIPEVFPIGYD |
| | | | GIAFIVNRSNPDSCI |
| | | | TVDDVKKILQGKIAK |
| | | | WNQLNPKNNRGSIEV |
| | | | VFDNKASATLHYVVD |
| | | | SILGGKNIKSENIVA |
| | | | AKNSKSVIDYVNKTP |
| | | | NAIGVIGSNWLNDHR |
| | | | DTTNTTFKKDVTVAS |
| | | | ISKATVASPSNSWQP |
| | | | YQAYLLDGRYPFVRT |
| | | | IYALLADPHKALPYA |
| | | | FANYIANPIGQMIIF |
| | | | KAGLLPYRGNINIRE |
| | | | VEVKNQ |
| 30 | Bifunctional_ | P9WHM7 | MAGTKRIKTALISVF |
| | purine_ | | HKDGLDDLLKKLDEE |
| | biosynthesis_ | | GVQFLSTGGTQQFIE |
| | protein_ | | SLGYECQKVEDVTSY |
| | PurH | | PSILGGRVKTLHPKI |
| | | | FGGILARRDNEEDQK |
| | | | QMVEYTIPAIDLVIV |
| | | | DLYPFEQTVASGASA |
| | | | QDIIEKIDIGGISLI |
| | | | RAGAKNFKDVVIVPS |
| | | | KAEYPVLLQLLNTKG |
| | | | AETEIEDRKMFAERA |
| | | | FGVSSHYDTAIHSWF |
| | | | AAE |
| 31 | Multidrug_ | P0AE06 | MEEEKGGRIGQRPYI |
| | efflux_ | | LKIITERNYIIIIDM |
| | pump_ | | KKAKILLFVTALVAV |
| | subunit_ | | LTSCGGGQKGLPTSD |
| | AcrA | | EYPVITIGASNAQLK |
| | | | TTYPATIKGVQDVEV |
| | | | RPKVSGFITKLNIHE |
| | | | GEYVHAGQVLFVIDN |
| | | | STYQAAVRQAQAQVN |
| | | | SAQSAVAQAKANVVQ |
| | | | ANASLNSANAQAATS |
| | | | RLTYNNSQNLYNNKV |
| | | | IGDYELQSAKNTYET |
| | | | AQASVRQAQSGLASA |
| | | | QAAVKQAEAGVRQAQ |
| | | | AMLSTAKDNLGFCYV |
| | | | KSPASGYVGSLPFKE |
| | | | DALVSASSAQPVTTI |
| | | | SNTSTIEVYFSMTEA |
| | | | DVLKLSRTDDGLSNA |
| | | | IKKFPAVSLLLADGS |
| | | | TYNHEGAIVKTSGMI |
| | | | DATTGTINVIARFPN |
| | | | PEHLLKSGGSGKIVI |
| | | | AKNNNRALLIPQEAV |
| | | | TQVQNKMFVYKVDAK |
| | | | DKVHYSEITVDPQND |
| | | | GINYIVTSGLKMGER |
| | | | IVSKGVSSLEDGAKI |
| | | | KALTPAEYEEAIKKA |
| | | | EKLGENQSSASGFLK |
| | | | TMKGDSK |
| 32 | Cell_division_ | Q81X30 | MAKRRNKARSHHSLQ |
| | protein_ | | WTLCISTAMVLILIG |
| | FtsX | | MVVLTVFTSRNLSSY |
| | | | VKENLTVTMILQPDM |
| | | | STEESAALCQRIRSL |
| | | | HYINSLNFISKEQAL |
| | | | KEGTRELGANPAEFA |
| | | | GQNPFTGEIELQLKA |
| | | | NYANNDSIKNIEREL |
| | | | RTYRGVSDITYPQNL |
| | | | VESVNHTLGKISLVL |
| | | | LVIAILLTIVSFSLM |
| | | | NNTIRLSIYARRFSI |
| | | | HTMKLVGASWGFIRA |
| | | | PFLRRAVMEGLVSAL |
| | | | LAIAVLGVGLCLLYD |
| | | | YEPDITKVLSWDVLV |
| | | | ITAGVMLAFGVLIAT |
| | | | FCSWLSVNKFLRMKA |
| | | | GDLYKI |
| 33 | Fe(2+)_ | Q9PMQ9 | MKLSDLKTGETGVIV |
| | Transporter_ | | KVLGHGGFRKRIIEM |
| | FeoB | | GFIQGKQVEVLLNAP |
| | | | LRDPVKYKIMGYEVS |
| | | | LRHSEADQIEVISAE |
| | | | EARQLEQAKADNEPQ |
| | | | QGALSNNIPDESDHA |
| | | | LTPFELTDAANRKSK |
| | | | VINVALVGNPNCGKT |
| | | | SLFNFASGAHERVGN |
| | | | YSGVTVDAKVGRANY |
| | | | EGYEFHLVDLPGTYS |
| | | | LSAYSPEELYVRKQL |
| | | | VEKTPDVVINVIDAS |
| | | | NLERNLYLTTQLIDM |
| | | | HVRMVCALNMFDETE |
| | | | QRGDNIDYQKISELF |
| | | | GIPMVPTVFTNGRGV |
| | | | KELFHQVIAVYEGKE |
| | | | DETSQFRHIHINHGH |
| | | | ELEGGIKNIQEHLRA |
| | | | YPDICQRYSTRYLAI |
| | | | KLLEHDKDVEELIKP |
| | | | LKDSDEIFKHRDIAA |
| | | | QRVKEETGNESETAI |
| | | | MDAKYGFIHGALEEA |
| | | | DYSTGQKKDTYQTTH |
| | | | FIDQILTNKYFGFPI |
| | | | FFLILFIMFTATFVI |
| | | | GQYPMDWIDGGVSWL |
| | | | GDFISSNMPDGPVKD |
| | | | MLVDGIIGGVGAVIV |
| | | | FLPQILILYFFISYM |
| | | | EDSGYMARAAFIMDK |
| | | | LMHKMGLHGKSFIPL |
| | | | IMGFGCNVPAVMATR |
| | | | TIESRRSRLVTMLIL |
| | | | PLMSCSARLPIYVMI |
| | | | TGSFFALKYRSLAML |
| | | | SLYVIGILMSVIMSR |
| | | | VFSRFLVKGEDTPFV |
| | | | MELPPYRFPTWKAIG |
| | | | RHTWEKGKQYLKKMG |
| | | | GIILVASIIVWALGY |
| | | | FPLPDKPDMGQQERQ |
| | | | EHSFIGQIGFLAVEP |
| | | | VFRPQGFNWKLDVGL |
| | | | LAGVGAKEIVASTMG |
| | | | VLYSNDDSFKDDNSF |
| | | | SSEGGKYVKLHKQIT |
| | | | QDVANLHGVSYNEAE |
| | | | PIATLTAFCFLLFVL |
| | | | LYFPCIATIAAIKGE |
| | | | TGSWGWALFAAGYTT |
| | | | LLAWVVSAIVFQVGM |
| | | | LFIG |
| 34 | Pneumolysin | Q04IN8 | MKKNLLKAVLPASLA |
| | | | LFAVTFGSCSQDGQL |
| | | | TGTKEDTGERVLDNT |
| | | | REIQNYLRTLPLAPM |
| | | | MSRASDPVPSDDGTT |
| | | | VPVDEGTSKTEEKGV |
| | | | LNGIPGSWVKTTRRY |
| | | | KMTQAFDESFLFDPT |
| | | | SDIVYPGCVLKGGTI |
| | | | ANGTYAIITSHETGD |
| | | | VTFSINLSPANPQEA |
| | | | RETSATVHNIRKSEY |
| | | | QEVWNKWANMQWKES |
| | | | PITTIESVEKINSQE |
| | | | ELATKLGVAVNSPVA |
| | | | NGSLNFGFNFNKKKN |
| | | | HILARLIQKYFSVST |
| | | | DAPKKGNIFESIDKE |
| | | | ALDGYQPVYISNINY |
| | | | GRIIYLSVESDEDEK |
| | | | VVDEAINFAMNQIKG |
| | | | VDVSVSADQSLHYRK |
| | | | VLANCDIRITVLGGG |
| | | | QTIQKEVLKGDIDS |
| | | | FQRFLNADIPMEQMS |
| | | | PISFSLRYAVDNSQA |
| | | | RVVTSNEFTVTQRDF |
| | | | VPEFKKVRMQLQVLG |
| | | | FSGTNTGPFPNLDRE |
| | | | AGLWGSISLSLNGQD |
| | | | NELVKISQSNPFFFN |
| | | | YREKKETMHPIGFGG |
| | | | IVTVEFDKDPNESLE |
| | | | DFVDHQKMTFVSDLH |
| | | | STRSIYNYNFGRTTF |
| | | | THTLGTLYTKYKGDD |
| | | | PIFVLESNNKNVKIH |
| | | | TYVKVLDMKFFN |
| 35 | Cluster: | G6AG77 | MTKFIYAMSLFLLAA |
| | Uncharacterized | | ISIKAQPIQKTSGCL |
| | protein | | LHGSVVSSTDATAIA |
| | | | GATVRLYQLKKLVGG |
| | | | TVSDASGNFDVKCPS |
| | | | SGSLQLRITAVGFKE |
| | | | VDTTLNVPTVTPLSI |
| | | | YMRAGKHAMDEVTVT |
| | | | ASEKRGMTSTTVIGQ |
| | | | TAMEHLQPSSFADLL |
| | | | ALLPGGMTKIPALGS |
| | | | ANVITLREAGPPSSQ |
| | | | YATSSLGTKFVIDGQ |
| | | | AIGTDANMQYIAGSF |
| | | | QGDADNSRNHVSYGV |
| | | | DMREIPTDNIEKVEV |
| | | | VRGIPSVKYGELTSG |
| | | | LINITRKRSQSPLLL |
| | | | RLKADEYGKLVSVGK |
| | | | GFLLSGKWNLNVDGG |
| | | | LLDARKEPRNRFETY |
| | | | RRLTFSARLRRKWNL |
| | | | GERYVLEWSGATDYS |
| | | | LNIDNVKTDPEIQIH |
| | | | REDSYRSSYLKMGMN |
| | | | HRLLLRRKALVGLQS |
| | | | VSLAYSASLASDRIH |
| | | | QTEAVALQRDYVVPL |
| | | | AYEGGEYDGLFLPMQ |
| | | | YLCDYRVEGKPFYST |
| | | | LRGETEWLARTSFIS |
| | | | HHITAGGEFLLNKNY |
| | | | GRGQIFDITKPLHAS |
| | | | TARRPRSYKDIPATD |
| | | | ILSFYAEDKATMPIG |
| | | | KHQLTVMAGLRTTQM |
| | | | LNIPASYAVHGKLFT |
| | | | DTRVNVQWDFPSFLG |
| | | | FKSFVSGGLGMMTKM |
| | | | PTVLDLYPDYVYKDI |
| | | | TEMNYWDIRPAYKRI |
| | | | HIRTYKLNQVNPDLR |
| | | | PARNKKWEIRLGMDK |
| | | | GAHHFSVTYFHEDMK |
| | | | DGFRSTTTMRPFIYK |
| | | | RYDTSVINPSALTGP |
| | | | PSLASLPVVTDTLLD |
| | | | GYGRTENGSRITKQG |
| | | | IEFQYSSPRIPVIQT |
| | | | RITVNGAWFRTLYEN |
| | | | SIPLFRSAPNVVVGT |
| | | | VAIADRYAGYYMSTD |
| | | | KYDKQIFTSNFIFDS |
| | | | YVDKLGLILSATAEC |
| | | | FWMSNTKRPATSSTP |
| | | | MGYMDITGTVHPYVE |
| | | | ADQSDPYLRWLVLTG |
| | | | TAGQDMDYRERSYML |
| | | | VNFKATKRFGRHLSL |
| | | | SFFADRVFYVAPDYE |
| | | | VNGFIVRRTFSPYFG |
| | | | MEIGLKI |
| 36 | Cell_division_ | P0A9R7 | MLIDFKKVNIYQDER |
| | ATP-binding_ | | LILKDIDFQATEGEF |
| | protein_ | | IYLIGRVGSGKSSLL |
| | FtsE | | KTFYGELDIDQEDAE |
| | | | KAEVLGESVLDIKQK |
| | | | RIPALRRQMGIIFQD |
| | | | FQLLHDRSVAKNLKF |
| | | | VLQATGWKDKEKIKQ |
| | | | RIKEVLEQVGMIDKA |
| | | | AKMPSELSGGEQQRI |
| | | | AIARAFLNNPKIILA |
| | | | DEPTGNLDPETASNI |
| | | | VSILKDTCKNGTTVI |
| | | | MSTHNINLLSQFPGK |
| | | | VYR |
| | | | CMEQALVPVTNEAQT |
| | | | KDLEEDSTSVEPLIE |
| | | | PVLEEEAQAEDSKE |
| 37 | Di/tripeptide_ | P0C2U3 | MFENQPKALYALALA |
| | transporter | | NTGERFGYYTMIAVF |
| | | | ALFLRANFGLEPGTA |
| | | | GLIYSIFLGLVYFLP |
| | | | LIGGIMADKFGYGKM |
| | | | VTIGIIVMFAGYLFL |
| | | | SVPLGGGTVAFGAML |
| | | | AALLLISFGTGLFKG |
| | | | NLQVMVGNLYDTPEL |
| | | | ASKRDSAFSIFYMAI |
| | | | NIGALFAPTAAVKIK |
| | | | EWAETSLGYAGNDAY |
| | | | HFSFAVACVSLIVSM |
| | | | GIYYAFRSTFKHVEG |
| | | | GTKKTEKAAAAAVEE |
| | | | LTPQQTKERIVALCL |
| | | | VFAVVIFFWMAFHQN |
| | | | GLTLTYFADEFVSPT |
| | | | STGVQSMAFDWNLVM |
| | | | IVFIVYSIMALFQSK |
| | | | TTKAKGIACAVILAA |
| | | | IAVLAYKYMNVNGQV |
| | | | EVSAPIFQQFNPFYV |
| | | | VALTPISMAIFGSLA |
| | | | AKGKEPSAPRKIAYG |
| | | | MIVAGCAYLLMVLAS |
| | | | QGLLTPHEQKLAKAA |
| | | | GETVPFASANWLIGT |
| | | | YLVLTFGELLLSPMG |
| | | | ISFVSKVAPPKYKGA |
| | | | MMGGWFVATAIGNIL |
| | | | VSVGGYLWGDLSLTV |
| | | | VWTVFIVLCLVSASF |
| | | | MFLMMKRLEKVA |
| 38 | Calcium- | Q47910 | MKKILIFVAGLCMSL |
| | transportingATP | | AASAQIQRPKLVVGL |
| | ase | | VVDQMRWDYLYYYYN |
| | | | EYGTDGLRRLVDNGF |
| | | | SFENTHINYAPTVTA |
| | | | IGHSSVYTGSVPAIT |
| | | | GIAGNYFFQDDKNVY |
| | | | CCEDPNVKSVGSDSK |
| | | | EGQMSPHRLLASTIG |
| | | | DELQISNDFRSKVIG |
| | | | VALKDRASILPAGHA |
| | | | ADAAYWWDTSAGHFV |
| | | | TSTFYTDHLPQWVID |
| | | | FNEKNHTAPNFNIKT |
| | | | STQGVTMTFKMAEAA |
| | | | LKNENLGKGKETDML |
| | | | AVSISSTDAIGHVYS |
| | | | TRGKENHDVYMQLDK |
| | | | DLAHFLKTLDEQVGK |
| | | | GNYLLFLTADHGAAH |
| | | | NYNYMKEHRIPAGGW |
| | | | DYRQSVKDLNGYLQG |
| | | | KFGIAPVMAEDDYQF |
| | | | FLNDSLIAASGLKKQ |
| | | | QIIDESVEYLKKDPR |
| | | | YLYVFDEERISEVTM |
| | | | PQWIKERMINGYFRG |
| | | | RSGEIGVVTRPQVFG |
| | | | AKDSPTYKGTQHGQP |
| | | | FPYDTHIPFLLYGWN |
| | | | VKHGATTQQTYIVDI |
| | | | APTVCAMLHIQMPNG |
| | | | CIGTARNMALGN |
| 39 | Poly-beta-1,6-N- | Q5HKQ0 | MDRQVFQTDSRQRWN |
| | acetyl-D- | | RFKWTLRVLITIAIL |
| | glucosamine_ | | LGVVFVAMFALEGSP |
| | synthase | | QMPFRHDYRSVVSAS |
| | | | EPLLKDNKRAEVYKS |
| | | | FRDFFKEQKMHSNYA |
| | | | KVAARQHRFVGHTDN |
| | | | VTQKYIKEWTDPRMG |
| | | | IRSAWYVNWDKHAYI |
| | | | SLKNNLKNLNMVLPE |
| | | | WYFINPKTDRIEARI |
| | | | DQRALKLMRRAHIPV |
| | | | LPMLTNNYNSAFRPE |
| | | | AIGRIMRDSTKRMGM |
| | | | INELVAACKHNGFAG |
| | | | INLDLEELNINDNAL |
| | | | LVTLVKDFARVFHAN |
| | | | GLYVTQAVAPFNEDY |
| | | | DMQELAKYDDYLFLM |
| | | | AYDEYNAGSQAGPVS |
| | | | SQRWVEKATDWAAKN |
| | | | VPNDKIVLGMATYGY |
| | | | NWAQGQGGTTMSFDQ |
| | | | TMATALNAGAKVNFN |
| | | | DDTYNLNFSYQDEDD |
| | | | GTLHQVFFPDAVTTF |
| | | | NIMRFGATYHLAGFG |
| | | | LWRLGTEDSRIWKYY |
| | | | GKDLSWESAARMPIA |
| | | | KIMQLSGTDDVNFVG |
| | | | SGEVLNVTSEPHAGR |
| | | | IGIVLDKDNQLIIEE |
| | | | RYLSLPATYTVQRLG |
| | | | KCKEKQLVLTFDDGP |
| | | | DSRWTPKVLSILKHY |
| | | | KVPAAFFMVGLQIEK |
| | | | NIPIVKDVFNQGCTI |
| | | | GNHTFTHHNMIENSD |
| | | | RRSFAELKLTRMLIE |
| | | | SITGQSTILFRAPYN |
| | | | ADADPTDHEEIWPMI |
| | | | IASRRNYLFVGESID |
| | | | PNDWQQGVTADQIYK |
| | | | RVLDGVHQEYGHIIL |
| | | | LHDAGGDTREPTVTA |
| | | | LPRIIETLQREGYQF |
| | | | ISLEKYLGMSRQTLM |
| | | | PPIKKGKEYYAMQAN |
| | | | LSLAELIYHISDFLT |
| | | | ALFLVFLVLGFMRLV |
| | | | FMYVLMIREKRAENR |
| | | | RNYAPIDPLTAPAVS |
| | | | IIVPAYNEEVNIVRT |
| | | | ISNLKEQDYPSLKIY |
| | | | LVDDGSKDNTLQRVR |
| | | | EVFENDDKWIISKKN |
| | | | GGKASALNYGIAACS |
| | | | TDYIVCVDADTQLYK |
| | | | DAVSKLMKHFIADKT |
| | | | GKLGAVAGNVKVGNQ |
| | | | RNMLTYWQAIEYTTS |
| | | | QNFDRMAYSNINAIT |
| | | | VIPGAIGAFRKDVLE |
| | | | AVGGFTTDTLAEDCD |
| | | | LTMSINEHGYLIENE |
| | | | NYAVAMTEAPESLRQ |
| | | | FIKQRIRWCFGVMQT |
| | | | FWKHRASLFAPSKGG |
| | | | FGMWAMPNMLIFQYI |
| | | | IPTFSPIADVLMLFG |
| | | | LFSGNASQIFIYYLI |
| | | | FLLVDASVSIMAYIF |
| | | | EHESLWVLLWIIPQR |
| | | | FFYRWIMYYVLFKSY |
| | | | LKAIKGELQTWGVLK |
| | | | RTGHVKGAQTIS |
| 40 | ATP_synthase_ | P29707 | MSQINGRISQIIGPV |
| | subunit_beta,_ | | IDVYFDTKGENPEKV |
| | sodium_ | | LPNIYDALRVKKADG |
| | ion_specific | | QDLIIEVQQQIGEDT |
| | | | VRCVAMDNTDGLQRG |
| | | | LEWPTGSPIVMPAGE |
| | | | QIKGRMMNVIGQPID |
| | | | GMSALQMEGAYPIHR |
| | | | EAPKFEDLSTHKEML |
| | | | QTGIKVIDLLEPYMK |
| | | | GGKIGLFGGAGVGKT |
| | | | VLIMELINNIAKGHN |
| | | | GYSVFAGVGERTREG |
| | | | NDLIRDMLESGVIRY |
| | | | GEKFRKAMDEGKWDL |
| | | | SLVDSEELQKSQATL |
| | | | VYGQMNEPPGARASV |
| | | | ALSGLTVAEEFRDHG |
| | | | GKNGEAADIMFFIDN |
| | | | IFRFTQAGSEVSALL |
| | | | GRMPSAVGYQPTLAS |
| | | | EMGAMQERITSTKHG |
| | | | SITSVQAVYVPADDL |
| | | | TDPAPATTFTHLDAT |
| | | | TELSRKITELGIYPA |
| | | | VDPLGSTSRILDPLI |
| | | | VGKEHYDCAQRVKQL |
| | | | LQKYNELQDIIAILG |
| | | | MDELSDDDKLVVNRA |
| | | | RRVQRFLSQPFT |
| | | | VAEQFTGVKGVMVPI |
| | | | EETIKGFNAILNGEV |
| | | | DDLPEQAFLNVGTIE |
| | | | DVKEKAKQLLEATKA |
| 41 | Cluster: | G6AGX5 | MNPIYKIITSILFCV |
| | Uncharacterized | | LSINTMAQDLTGHVT |
| | protein | | SKADDKPIAYATVTL |
| | | | KENRLYAFTDEKGNY |
| | | | TIKNVPKGKYTVVFS |
| | | | CMGYASQTVVVMVNA |
| | | | GGATQNVRLAEDNLQ |
| | | | LDEVQVVAHRKKDEI |
| | | | TTSYTIDRKTLDNQQ |
| | | | IMTLSDIAQLLPGGK |
| | | | SVNPSLMNDSKLTLR |
| | | | SGTLERGNASFGTAV |
| | | | EVDGIRLSNNAAMGE |
| | | | TAGVSTRSVSASNIE |
| | | | SVEVVPGIASVEYGD |
| | | | LTNGVVKVKTRRGSS |
| | | | PFIVEGSINQHTRQI |
| | | | ALHKGVDLGGNVGLL |
| | | | NFSIEHARSFLDAAS |
| | | | PYTAYQRNVLSLRYM |
| | | | NVFMKKSLPLTLEVG |
| | | | LNGSIGGYNSKADPD |
| | | | RSLDDYNKVKDNNVG |
| | | | GNIHLGWLLNKRWIT |
| | | | NVDLTAAFTYADRLS |
| | | | ESYTNESSNATQPYI |
| | | | HTLTEGYNIAEDYDR |
| | | | NPSANIILGPTGYWY |
| | | | LRGFNDSKPLNYSLK |
| | | | MKANWSKAFGKFRNR |
| | | | LLVGGEWTSSMNRGR |
| | | | GTYYADMRYAPSWRE |
| | | | YRYDALPSLNNIAIY |
| | | | AEDKLSMDVNERQNA |
| | | | ELTAGIREDITSIPG |
| | | | SEYGSVGSFSPRMNA |
| | | | RYVFRFGQNSWLNSM |
| | | | TLHAGWGRSVKIPSF |
| | | | QVLYPSPSYRDMLAF |
| | | | ASTSDADNRSYYAYY |
| | | | TYPSMARYNANLKWQ |
| | | | RADQWDLGVEWRTKI |
| | | | ADVSLSFFRSKVSNP |
| | | | YMATDVYTPFTYKYT |
| | | | SPAMLQRSGIAVADR |
| | | | RFSIDPQTGIVTVSD |
| | | | ASGVKSPVTLGYEER |
| | | | NTYVTNTRYVNADAL |
| | | | QRYGLEWIVDFKQIK |
| | | | TLRTQVRLDGKYYHY |
| | | | KAQDETLFADVPVGL |
| | | | NTRQSDGRLYQYVGY |
| | | | YRGGAATTTNYTANA |
| | | | SASNGSVSGQVDLNA |
| | | | TITTHIPKIRLIVAL |
| | | | RLESSLYAFSRATSS |
| | | | RGYVVSSGNEYFGVP |
| | | | YDDKTENQTVIVYPE |
| | | | YYSTWDAPDVLIPFA |
| | | | EKLRWAETNDRGLFN |
| | | | DLAQLVVRTNYPYTL |
| | | | NPNRLSAYWSANLSV |
| | | | TKEIGRHVSVSFYAN |
| | | | NFFNTLSQVHSTQTG |
| | | | LETSLFGSGYVPSFY |
| | | | YGLSLRLKI |

In some embodiments, the Prevotella bacteria is a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25 or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25 or more) genes encoding proteins listed in Table 2. In some embodiments, Prevotella bacteria is free of all of the proteins listed in Table 2 and/or all of the genes encoding the proteins listed in Table 2.

TABLE 2
Other Prevotella proteins

| Seq. | | | |
|---|---|---|---|
| ID. | | Uniprot | Amino Acid |
| No. | Name | ID | Sequence |
| 42 | UDP-Gal: | Q03084 | MERIDISVLMAVYKK |
| | alpha-D- | | DNPAFLRESLESIFS |
| | GlcNAc- | | QTVEAAEVVLLEDGP |
| | Diphospho- | | LTDALYDVIKSYEAI |
| | undecaprenol | | YSTLKVVSYPENRGL |
| | | | GKTLNDGLLLCKYNL |
| | | | VARMDADDICKPNRL |
| | | | EMEYNWLKSHEDYDV |
| | | | IGSWVDEFTDNKTRV |
| | | | KSIRKVPEAYDEIKN |
| | | | YAQYRCPINHPTAMY |
| | | | RKAAVLAVGGYLTEY |
| | | | FPEDYFLWLRMLNNG |
| | | | SKFYNIQESLLWFRY |
| | | | SEETVAKRGGWAYAC |
| | | | DEVRILVRMLKMGYI |
| | | | PFHVFCQSVVIRFTT |
| | | | RVMPLPIRQRLYNLI |
| | | | RKT |
| 43 | ATP_synthase_ | A1B8P0 | MSQINGRISQIIGPV |
| | subunit_ | | IDVYFDTKGENPEKV |
| | beta | | LPKIHDALRVKRANG |
| | | | QDLIIEVQQHIGEDT |
| | | | VRCVAMDNTDGLQRN |
| | | | LEVVPTGSPIVMPAG |
| | | | DQIKGRMMNVIGQPI |
| | | | DGMEALSMEGAYPIH |
| | | | REAPKFEDLSTHKEM |
| | | | LQTGIKVIDLLEPYM |
| | | | KGGKIGLFGGAGVGK |
| | | | TVLIMELINNIAKGH |
| | | | NGYSVFAGVGERTRE |
| | | | GNDLIRDMLESGVIR |
| | | | YGEKFRKAMDEGKWD |
| | | | LSLVDQEELQKSQAT |
| | | | LVYGQMNEPPGARAS |
| | | | VALSGLTVAEEFRDH |
| | | | GGKNGEAADIMFFID |
| | | | NIFRFTQAGSEVSAL |
| | | | LGRMPSAVGYQPTLA |
| | | | SEMGTMQERITSTKH |
| | | | GSITSVQAVYVPADD |
| | | | LTDPAPATTFTHLDA |
| | | | TTELSRKITELGIYP |
| | | | AVDPLGSTSRILDPL |
| | | | IVGKDHYECAQRVKQ |
| | | | LLQHYNELQDIIAIL |
| | | | GMDELSDEDKLVVNR |
| | | | ARRVQRFLSQPFTVA |
| | | | EQFTGVKGVMVPIEE |
| | | | TIKGFNAILNGEVDD |
| | | | LPEQAFLNVGTIEDV |
| | | | KEKAKRLLEATK |
| 44 | Cell_ | O05779 | MPIGNGQKYQLTIIN |
| | division_ | | HTEIIMLIDYKKVNI |
| | ATP- | | YQDERLILKDVDFQA |
| | binding_ | | ETGEFIYLIGRVGSG |
| | protein_ | | KSSLLKTIYGELDID |
| | FtsE | | SEDAEKAVVLDESMP |
| | | | NIKRSRIPALRKQMG |
| | | | IIFQDFQLLHDRSVA |
| | | | KNLKFVLQATGWTSK |
| | | | QKIERRIEEVLAQVG |
| | | | MTDKKNKMPSELSGG |
| | | | EQQRIAIARALLNTP |
| | | | KIIIADEPTGNLDPE |
| | | | TAANIVSILKDSCQA |
| | | | GTTVIMSTHNINLID |
| | | | QFPGKVYRCHEGELH |
| | | | QLTDKKEVSELAEET |
| | | | APVETIDEPEQND |
| 45 | Hemin_ | Q56992 | MKRNILLFICLATSI |
| | transport_ | | LLLFGLNLTTGSVQI |
| | system_ | | PFADILDILCGRFIG |
| | permease_ | | KESWEYIILENRLPQ |
| | protein_HmuU | | TLTAILCGASLSVCG |
| | | | LMLQTAFRNPLAGPD |
| | | | VFGISSGAGLGVALV |
| | | | MLLLGGTVSTSIFTV |
| | | | SGFLAILTAAFVGAI |
| | | | AVTALILFLSTLVRN |
| | | | SVLLLIVGIMVGYVS |
| | | | SSAVSLLNFFASEEG |
| | | | VKSYMVWGMGNFGAV |
| | | | SMNHIPLFSILCLIG |
| | | | IIASFLLVKPLNILL |
| | | | LGPQYAESLGISTRQ |
| | | | IRNILLVWGLLTAIT |
| | | | TAFCGPISFIGLAIP |
| | | | HIARLLFRTENHQIL |
| | | | LPGIVLSGAAIALLC |
| | | | NFICYLPGESGIIPL |
| | | | NAVTPLIGAPIIIYV |
| | | | IIQRR |
| 46 | Hexuronate_ | O34456 | MKKYYPWVLVALLWF |
| | transporter | | VALLNYMDRQMLSTM |
| | | | QEAMKVDIAELNHAE |
| | | | AFGALMAVFLWIYGI |
| | | | VSPFAGIIADRVNRK |
| | | | WLVVGSIFVWSAVTY |
| | | | LMGYAESFDQLYWLR |
| | | | AFMGISEALYIPAAL |
| | | | SLIADWHEGKSRSLA |
| | | | IGIHMTGLYVGQAVG |
| | | | GFGATLAAMFSWHAA |
| | | | FHWFGIIGIVYSLVL |
| | | | LLFLKENPKHGQKSV |
| | | | LQGETKPSKNPFRGL |
| | | | SIVFSTWAFWVILFY |
| | | | FAVPSLPGWATKNWL |
| | | | PTLFANSLDIPMSSA |
| | | | GPMSTITIAVSSFIG |
| | | | VIMGGVISDRWVQRN |
| | | | LRGRVYTSAIGLGLT |
| | | | VPALMLLGFGHSLVS |
| | | | VVGAGLCFGIGYGMF |
| | | | DANNMPILCQFISSK |
| | | | YRSTAYGIMNMTGVF |
| | | | AGAAVTQVLGKWTDG |
| | | | GNLGNGFAILGGIWL |
| | | | ALVLQLSCLKPTTDN |
| | | | ME |
| 47 | 1,4-alpha- | P9WN45 | MVTKKTTTKKAPVKK |
| | Glucan_ | | TSAKTTKVKEPSHIG |
| | Branching_ | | LVKNDAYLAPYEDAI |
| | Enzyme_GlgB | | RGRHEHALWKMNQLT |
| | | | QNGKLTLSDFANGHN |
| | | | YYGLHQTADGWVFRE |
| | | | WAPNATEIYLVGDFN |
| | | | GWNEQEAYQCHRIEG |
| | | | TGNWELTLPHDAMQH |
| | | | GQYYKMRVHWEGGEG |
| | | | ERIPAWTQRVVQDEA |
| | | | SKIFSAQVWAPAEPY |
| | | | VWEKKTFKPQTSPLL |
| | | | IYECHIGMAQDEEKV |
| | | | GTYNEFREKVLPRII |
| | | | KDGYNAIQIMAIQEH |
| | | | PYYGSFGYHVSSFFA |
| | | | ASSRFGTPEELKALI |
| | | | DEAHKNGIAVIMDIV |
| | | | HSHAVKNEVEGLGNL |
| | | | AGDPNQYFYPGERHE |
| | | | HPAWDSLCFDYGKDE |
| | | | VLHFLLSNCKYWLEE |
| | | | YHFDGFRFDGVTSML |
| | | | YYSHGLGEAFCNYAD |
| | | | YFNGHQDDNAICYLT |
| | | | LANCLIHEVNKNAVT |
| | | | IAEEVSGMPGLAAKF |
| | | | KDGGYGFDYRMAMNI |
| | | | PDYWIKTIKELPDEA |
| | | | WKPSSIFWEIKNRRS |
| | | | DEKTISYCESHDQAL |
| | | | VGDKTIIFRLVDADM |
| | | | YWHFRKGDETEMTHR |
| | | | GIALHKMIRLATIAA |
| | | | INGGYLNFMGNEFGH |
| | | | PEWIDFPREGNGWSH |
| | | | KYARRQWNLVDNEEL |
| | | | CYHLLGDFDRKMLE |
| | | | VITSEKKFNETPIQE |
| | | | IWHNDGDQILAFSRG |
| | | | ELVFVFNFSPSHSYS |
| | | | DYGFLVPEGSYNVVL |
| | | | NTDAREFGGFGFADD |
| | | | TVEHFTNSDPLYEKD |
| | | | HKGWLKLYIPARSAV |
| | | | VLRKK |
| 48 | Cluster: YihY | D9RW24 | MKIDIERIKYFLTVG |
| | family protein | | MFMKTEHSSKRRNML |
| | | | IRQFQKFYLTVKFFF |
| | | | VRDHAASTAQLSFST |
| | | | IMAIVPIASMIFAIA |
| | | | NGFGFGQFLEKQFRE |
| | | | MLSAQPEAATWLLKL |
| | | | TQSYLVHAKTGLFIG |
| | | | IGLMIMLYSVFSLIR |
| | | | TVETTFDNIWQVKDS |
| | | | RPISRIVIDYTALMF |
| | | | LVPISIIILSGLSIY |
| | | | FYSFVENLNGLRFLG |
| | | | TIASFSLRYLVPWAI |
| | | | LTLMFIVLYVFMPNA |
| | | | KVKITKTVAPAMIAS |
| | | | IAMLCLQAVYIHGQI |
| | | | FLTSYNAIYGSFAAL |
| | | | PLFMLWILASWYICL |
| | | | FCAELCYFNQNLEYY |
| | | | ECLIDTEDICHNDLL |
| | | | ILCATVLSHICQRFA |
| | | | NDQKPQTALQIKTET |
| | | | HIPIRVMTDILYRLK |
| | | | EVNLISENFSPTSDE |
| | | | VTYTPTHDTNNITVG |
| | | | EMIARLESTPASDFA |
| | | | LLGFSPKKAWNHDIY |
| | | | DRVGSIREIYLNELK |
| | | | SINIKELISYSEN |
| 49 | Capsule_ | P19579 | MMKRPSIARVVKVII |
| | biosynthesis_ | | CLLTPILLSFSGIGD |
| | protein_ | | NDIDKKKSTSKEVDD |
| | CapA | | TLRIVITGDLLLDRG |
| | | | VRQKIDMAGVDALFS |
| | | | PTIDSLFHSSNYVIA |
| | | | NLECPVTKIRERVFK |
| | | | RFIFRGEPEWLPTLR |
| | | | RHGITHLNLANNHSI |
| | | | DQGRNGLLDTQEQIK |
| | | | KAGMIPIGAGKNMEE |
| | | | AAEPVLISTSPRHVW |
| | | | VISSLRLPLENFLYL |
| | | | PQKPCVSQESIDSLI |
| | | | MRVKRLRATDKNCYI |
| | | | LLILHWGWEHHFRAT |
| | | | PQQREDAHKLIDAGA |
| | | | DAIVGHHSHTLQTIE |
| | | | TYRGKPIYYGIGNFI |
| | | | FDQRKPMNSRACLVE |
| | | | LSITAEKCKAKALPI |
| | | | EIKNCTPYLSK |
| 50 | Peptidoglycan_ | B5ZA76 | MILLSFDTEEFDVPR |
| | deacetylase | | EHGVDFSLEEGMKVS |
| | | | IEGTNRILDILKANN |
| | | | VCATFFCTGNFAELA |
| | | | PEVMERIKNEGHEVA |
| | | | CHGVDHWQPKPEDVF |
| | | | RSKEIIERVTGVKVA |
| | | | GYRQPRMFPVSDEDI |
| | | | EKAGYLYNSSLNPAF |
| | | | IPGRYMHLTTSRTWF |
| | | | MQGKVMQIPASVSPH |
| | | | LRIPLFWLSMHNFPE |
| | | | WFYLRLVRQVLRHDG |
| | | | YFVTYFHPWEFYDLK |
| | | | SHPEFKMPFIIKNHS |
| | | | GHELEQRLDRFIKAM |
| | | | KADKQEFITYVDFVN |
| | | | RQKK |
| 51 | Fumarate_ | P0AC47 | MAKNISFTIKYWKQN |
| | Reductase_ | | GPQDQGHFDTHEMKN |
| | iron- | | IPDDTSFLEMLDILN |
| | sulfur_ | | EELIAAGDEPFVFDH |
| | subunit | | DCREGICGMCSLYIN |
| | | | GTPHGKTERGATTCQ |
| | | | LYMRRFNDGDVITVE |
| | | | PWRSAGFPVIKDCMV |
| | | | DRTAFDKIIQAGGYT |
| | | | TIRTGQAQDANAILI |
| | | | SKDNADEAMDCATCI |
| | | | GCGACVAACKNGSAM |
| | | | LFVSSKVSQLALLPQ |
| | | | GKPEAAKRAKAMVAK |
| | | | MDEVGFGNCTNTRAC |
| | | | EAVCPKNEKIANIAR |
| | | | LNREFIKAKFAD |
| 52 | Serine/ | P9WI71 | MSENKLSTNEQAQTA |
| | threonine- | | DAPVKASYTEYKVIP |
| | protein_ | | SQGYCMIVKCRKGDQ |
| | kinase_Pk | | TVVLKTLKEEYRERV |
| | nH | | LLRNALKREFKQCQR |
| | | | LNHSGIVRYQGLVEV |
| | | | DGYGLCIEEEYVEGR |
| | | | TLQAYLKENHTDDEK |
| | | | IAIINQIADALRYAH |
| | | | QQGVIHRNLKPSNVL |
| | | | VTTQGDYVKLIDFSV |
| | | | LSPEDVKPTAETTRF |
| | | | MAPEMKDETLTADAT |
| | | | ADIYSLGTIMKVMGL |
| | | | TLAYSEVIKRCCAFK |
| | | | RSDRYSNVDELLADL |
| | | | NNEGSSFSMPKIGKG |
| | | | TVVLGLIIAVVIGIG |
| | | | ALLYNYGGALIDQVG |
| | | | KIDVSSVFSSDAETA |
| | | | PEDTVKVNTAEQSDS |
| | | | LSTEAEAPAIGKLAF |
| | | | MNRMKPALYKDLDNI |
| | | | FEKNSADKAKLTKAI |
| | | | KTYYRGLIQANDTLD |
| | | | NEQRAEVDRVFGDYV |
| | | | KQKKAALN |
| 53 | Carboxy- | O34666 | MRKYICLLLFYLFTF |
| | terminal_ | | LPLSAQQGNDSPLRK |
| | proccssing_ | | LQLAEMAIKNFYVDS |
| | protease_ | | VNEQKLVEDGIRGML |
| | CtpA | | EKLDPHSTYTDAKET |
| | | | KAMNEPLQGDFEGIG |
| | | | VQFNMIEDTLVVIQP |
| | | | VVNGPSQKVGILAGD |
| | | | RIVSVNDSTIAGVKM |
| | | | ARIDIMKMLRGKKGT |
| | | | KVKLGVVRRGVKGVL |
| | | | TFVVTRAKIPVHTIN |
| | | | ASYMIRPNVGYIRIE |
| | | | SFGMKTHDEFMSAVD |
| | | | SLKKKGMKTLLLDLQ |
| | | | DNGGGYLQSAVQISN |
| | | | EFLKNNDMIVYTEGR |
| | | | RARRQNFKAIGNGRL |
| | | | QDVKVYVLVNELSAS |
| | | | AAEIVTGAIQDNDRG |
| | | | TVVGRRTFGKGLVQR |
| | | | PFDLPDGSMIRLTIA |
| | | | HYYTPSGRCIQKPYT |
| | | | KGDLKDYEMDIEKRF |
| | | | KHGELTNPDSIQFSD |
| | | | SLKYYTIRKHRVVYG |
| | | | GGGIMPDNFVPLDTT |
| | | | KFTRYHRMLAAKSII |
| | | | INAYLKYADANRQAL |
| | | | KAQYSSFDAFNKGYV |
| | | | VPQSLLDEIVAEGKK |
| | | | EKIEPKDAAELKATL |
| | | | PNIALQIKALTARDI |
| | | | WDMNEYFRVWNTQSD |
| | | | IVNKAVALATGK |
| 54 | Cluster: | D9RRG3 | MKLTEQRSSMLHGVL |
| | Uncharacterized | | LITLFACAAFYIGDM |
| | protein | | GWVKALSLSPMVVGI |
| | | | ILGMLYANSLRNNLP |
| | | | DTWVPGIAFCGKRVL |
| | | | RFGIILYGFRLTFQD |
| | | | VVAVGFPAIIVDAII |
| | | | VSGTILLGVLVGRLL |
| | | | KMDRSIALLTACGSG |
| | | | ICGAAAVLGVDGAIR |
| | | | PKPYKTAVAVATVVI |
| | | | FGTLSMFLYPILYRA |
| | | | GIFDLSPDAMGIFAG |
| | | | STIHEVAFTVVGAGN |
| | | | AMGAAVSNSAIIVKM |
| | | | IRVMMLVPVLLVIAF |
| | | | FVAKNVAERDDEAGG |
| | | | SRKINIPWFAILFLV |
| | | | VIG |
| | | | FNSLNLLPKELVDFI |
| | | | NTLDTFLLTMAMSAL |
| | | | GAETSIDKFKKAGFK |
| | | | PFLLAAILWCWLIGG |
| | | | GYCLAKYLVPVLGVA |
| | | | C |
| 55 | Cluster: Cna | X6Q2J4 | MNKQFLLAALWLSPL |
| | protein B-type | | GLYAHKANGIGAVTW |
| | domain protein | | KNEAPKERMIRGIDE |
| | | | DKTHQRFTLSGYVKD |
| | | | RNGEPLINATIYDLT |
| | | | TRQGTMTNAYGHFSL |
| | | | TLGEGQHEIRCSYVG |
| | | | YKTLIETIDLSANQN |
| | | | HDIILQNEAQLDEVV |
| | | | VTTDLNSPLLKTQTG |
| | | | KLSLSQKDIKTEYAL |
| | | | LSSPDVIKTLQRTSG |
| | | | VADGMELASGLYVHG |
| | | | GNGDENLFLLDGTPL |
| | | | YHTNHSLGLFSSFNA |
| | | | DVVKNVDFYKSGFPA |
| | | | RYGGRLSSVIDVRTA |
| | | | DGDLYKTHGSYRIGL |
| | | | LDGAFHIGGPIRKGK |
| | | | TSYNFGLRRSWMDLL |
| | | | TRPAFAIMNHKSDNE |
| | | | DKLSMSYFFHDLNFK |
| | | | LTNIFNERSRMSLSV |
| | | | YSGEDRLDAKDEWHS |
| | | | NNSSGYNDVDIYVNR |
| | | | FHWGNFNAALDWNYQ |
| | | | FSPKLFANFTAVYTH |
| | | | NRSTVSSSDEWRFTR |
| | | | PGEKEQLTLTSHGYR |
| | | | SSIDDIGYRAAFDFR |
| | | | PSPRHHIRFGQDYTY |
| | | | HRFQPQTYNRFDNYQ |
| | | | TNSEAKADTIATHSY |
| | | | NKNVAHQLTFYAEDE |
| | | | MTLNEKWSLNGGVNA |
| | | | DVFHISGKTFATLSP |
| | | | RLSMKFQPTERLSLK |
| | | | ASYTLMSQFVHKIAN |
| | | | SFLDLPTDYWVPTTA |
| | | | RLHPMRSWQVAAGAY |
| | | | MKPNKHWLLSLEAYY |
| | | | KRSSHILQYSSWAGL |
| | | | EPPAANWDYMVMEGD |
| | | | GRSYGVELDADYNVS |
| | | | NLTLHGSYTLSWTQK |
| | | | KFDDFYDGWYYDKFD |
| | | | NRFIKLTLTGRWNIT |
| | | | KKIAAFAAWTFRTGN |
| | | | RMTIPTQYIGLPDVP |
| | | | AQEQGGLTFNSSDDN |
| | | | TLNFAYEKPNNVILP |
| | | | AYHRLDIGFDFHHTT |
| | | | KKGFIERIWNLSFYN |
| | | | AYCHLNSLWVRVKID |
| | | | SNNQMKIRNIAFIPV |
| | | | IPSFSYTFKF |
| 56 | Poly-beta- | P75905 | MSKQVFQTDSRQRWS |
| | 1,6-N-acetyl- | | YFKWTLRVILTILSL |
| | D-glucosamine_ | | LGIVFLAMFALEGSP |
| | synthase | | QMPFRHDYRNAVTAA |
| | | | SPYTKDNKTAKLYKS |
| | | | FRDFFKEKKMHNNYA |
| | | | KATIKKQRFIGKADS |
| | | | VTQKYFREWDDPRIG |
| | | | VRSAWYVNWDKHAYI |
| | | | SLKNNIKHLNMVLPE |
| | | | WFFINPKTDKVEYRI |
| | | | DKQALRLMRRTGIPV |
| | | | LPMLTNNYNSDFHPE |
| | | | AIGRIMRDEKKRMAL |
| | | | INEMVRTCRHYGFAG |
| | | | INLDLEELNIQDNDL |
| | | | LVELLKDFSRVFHAN |
| | | | GLYVTQAVAPFNEDY |
| | | | NMQELAKYNDYLFLM |
| | | | AYDEHNIESQPGAVS |
| | | | SQRWVEKATDWAAKN |
| | | | VPNDKIVLGMATYGY |
| | | | DWANGEGGTTVSFDQ |
| | | | TMAIAQDADAKVKFD |
| | | | DDTYNVNFSYQNTDD |
| | | | GKIH |
| | | | HVFFTDAATTFNIMR |
| | | | FGAEYHLAGYGLWRL |
| | | | GTEDKRIWRFYGKDM |
| | | | SWENVARMSVAKLMQ |
| | | | LNGTDDWFVGSGEVL |
| | | | EVTTEPHPGDISIRI |
| | | | DKDNRLISEEYYRAL |
| | | | PSTYTIQRLGKCKDK |
| | | | QLVITFDDGPDSRWT |
| | | | PTVLSTLKKYNVPAA |
| | | | FFMVGLQMEKNLPLV |
| | | | KQVYEDGHTIGNHTF |
| | | | THHNMIENSDRRSYA |
| | | | ELKLTRMLIESVTGH |
| | | | STILFRAPYNADADP |
| | | | TEHEEIWPMIVASRR |
| | | | NYLFVGESIDPNDWE |
| | | | PNVTSDQIYQRVIDG |
| | | | VHHEDGHIILLHDAG |
| | | | GSSRKPTLDALPRII |
| | | | ETLQHEGYQFISLEQ |
| | | | YLGMGKQTLMPEINK |
| | | | GKAYYAMQTNLWLAE |
| | | | MIYHVSDFLTALFLV |
| | | | FLALGMMRLIFMYVL |
| | | | MIREKRAENRRNYAP |
| | | | IDAATAPAVSIIVPG |
| | | | YNEEVNIVRTITTLK |
| | | | QQDYPNLHIYFVDDG |
| | | | SKDHTLERVHEAFDN |
| | | | DDTVTILAKKNGGKA |
| | | | SALNYGIAACRSEYV |
| | | | VCIDADTQLKNDAVS |
| | | | RLMKHFIADTEKRVG |
| | | | AVAGNVKVGNQRNML |
| | | | TYWQAIEYTSSQNFD |
| | | | RMAYSNINAITVVPG |
| | | | AIGAFRKEVIEAVGG |
| | | | FTTDTLAEDCDLTMS |
| | | | INEHGYIIENENYAV |
| | | | ALTEAPETLRQFVKQ |
| | | | RIRWCFGVMQAFWKH |
| | | | RSSLFAPSKKGFGLW |
| | | | AMPNMLIFQYIIPTF |
| | | | SPLADVLMLIGLFTG |
| | | | NALQIFFYYLIFLVI |
| | | | DASVSIMAYIFEGER |
| | | | LWVLLWVIPQRFFYR |
| | | | WIMYYVLFKSYLKAI |
| | | | KGELQTWGVLKRTGH |
| | | | VKG |
| 57 | Cell_ | O34876 | MAKKRNKARSRHSLQ |
| | division_ | | VVTLCISTAMVLMLI |
| | protein_ | | GIVVLTGFTSRNLSS |
| | FtsX | | YVKENLTITMILQPD |
| | | | MNTEESAALCERIRT |
| | | | LHYINSLNFISKEQA |
| | | | LKDGTKELGANPAEF |
| | | | AGENPFTGEIEVQLK |
| | | | ANYANNDSIRNIVQQ |
| | | | LRTYRGVSDITYPQS |
| | | | LVESVNQTLGKISLV |
| | | | LLVIAVLLTIISFSL |
| | | | INNTIRLSIYAHRFS |
| | | | IHTMKLVGGSWSFIR |
| | | | APFLRRAVLEGLVSA |
| | | | LLAIAVLGIGICLLY |
| | | | EKEPEITKLLSWDAL |
| | | | IITAIVMLAFGVIIA |
| | | | TFCAWLSVNKFLRMK |
| | | | AGDLYKI |
| 58 | UDP-2,3- | P44046 | MKNIYFLSDAHLGSL |
| | diacylglucosamine_ | | AIDHRRTHERRLVRF |
| | hydrolase | | LDSIKHKAAAVYLLG |
| | | | DMFDFWNEYKYVVPK |
| | | | GFTRFLGKISELTDM |
| | | | GVEVHFFTGNHDLWT |
| | | | YGYLEKECGVILHRK |
| | | | PITTEIYDKVFYLAH |
| | | | GDGLGDPDPMFRFLR |
| | | | KVFHNRFCQRLLNFF |
| | | | HPWWGMQLGLNWAKR |
| | | | SRLKRKDGKEVPYLG |
| | | | EDKEYLVQYTKEYMS |
| | | | THKDIDYYIYGHRHI |
| | | | ELDLTLSRKARLLIL |
| | | | GDWIWQFTYAVFDGE |
| | | | HMFLEEYVEGESKP |
| 59 | Poly-beta- | P75905 | MVGLDVLCYFIFIAK |
| | 1,6-N- | | GREKECYFERIIYQI |
| | acetyl-D- | | TCHSRTKCYLCNIMK |
| | glucosamine_ | | YSIIVPVFNRPDEVE |
| | synthase | | ELLESLLSQEEKDFE |
| | | | VVIVEDGSQIPCKEV |
| | | | CDKYADKLDLHYYSK |
| | | | ENSGPGQSRNYGAER |
| | | | AKGEYLLILDSDVVL |
| | | | PKGYICAVSEELKRE |
| | | | PADAFGGPDCAHESF |
| | | | TDTQKAISYSMTSFF |
| | | | TTGGIRGGKKKLDKF |
| | | | YPRSFNMGIRRDVYQ |
| | | | ELGGFSKMRFGEDID |
| | | | FSIRIFKAGKRCRLF |
| | | | PEAWVWHKRRTDFRK |
| | | | FWKQVYNSGIARINL |
| | | | YKKYPESLKLVHLLP |
| | | | MVFTVGTALLVLMIL |
| | | | FGLFLQLFPIINVFG |
| | | | SVFIMMGLMPLVLYS |
| | | | VIICVDSTMQNNSLN |
| | | | IGLLSIEAAFIQLTG |
| | | | YGCGFISAWWKRCVC |
| | | | GMDEFAAYEKNFYK |
| 60 | Enolase | Q8DTS9 | MKIEKVHAREIMDSR |
| | | | GNPTVEVEVTLENGV |
| | | | MGRASVPSGASTGEN |
| | | | EALELRDGDKNRFLG |
| | | | KGVLKAVENVNNLIA |
| | | | PALKGDCVLNQRAID |
| | | | YKMLELDGTPTKSKL |
| | | | GANAILGVSLAVAQA |
| | | | AAKALNIPLYRYIGG |
| | | | ANTYVLPVPMMNIIN |
| | | | GGAHSDAPIAFQEFM |
| | | | IRPVGAPSEKEGIRM |
| | | | GAEVFHALAKLLKKR |
| | | | GLSTAVGDEGGFAPK |
| | | | FDGIEDALDSIIQAI |
| | | | KDAGYEPGKDVKIAM |
| | | | DCAASEFAVCEDGKW |
| | | | FYDYRQLKNGMPKDP |
| | | | NGKKLSADEQIAYLE |
| | | | HLITKYPIDSIEDGL |
| | | | DENDWENWVKLTSAI |
| | | | GDRCQLVGDDLFVTN |
| | | | VKFLEKGIKMGAANS |
| | | | ILIKVNQIGSLTETL |
| | | | EAIEMAHRHGYTTVT |
| | | | SHRSGETEDTTIADI |
| | | | AVATNSGQIKTGSMS |
| | | | RTDRMAKYNQLIRIE |
| | | | EELGACAKYGYAKLK |
| 61 | Outer_ | Q8G0Y6 | MKKLFTIAMLLGVTL |
| | membrane_ | | GIHAQEVYSLQKCRE |
| | efflux_ | | LALQNNRQLKVSRMT |
| | protein_BepC | | VDVAENTRKAAKTKY |
| | | | LPRVDALAGYQHFSR |
| | | | EISLLSDDQKNAFSN |
| | | | LGTNTFGQLGGQIGQ |
| | | | NLTSLAQQGILSPQM |
| | | | AQQLGQLFSNVATPL |
| | | | TQVGNNIGQSINDAF |
| | | | RSNTKNVYAGGIVVN |
| | | | QPIYMGGAIKAANDM |
| | | | AAIGEQVAQNNISLK |
| | | | RQLVLYGVDNAYWLA |
| | | | ISLKKKEALAIRYRD |
| | | | LAQKLNEDVKKMIRE |
| | | | GVATRADGLKVEVAV |
| | | | NTADMQIARIQSGVS |
| | | | LAKMALCELCGLELN |
| | | | GDIPLSDEGDADLPP |
| | | | TPSTQFDNYTVSSSD |
| | | | TTGLNEARPELRLLQ |
| | | | NAVDLSIQNTKLIRS |
| | | | LYMPHVLLTAGYSVS |
| | | | NPNLFNGFQKRFTDL |
| | | | WNIGITVQVPVWNWG |
| | | | ENKYKVRASKTATTI |
| | | | AQLEMDDVRKKIDLE |
| | | | IEQNRLRLKDANKQL |
| | | | ATSQKNMAAAEENLR |
| | | | CANVGFKEGVMTVTE |
| | | | VMAAQ |
| | | | TAWQTSRMAIIDAEI |
| | | | SVKLAQTGLQKALGG |
| | | | L |
| 62 | Phosphoethanol- | Q7CPC0 | MKRTFVTKMVKPIEE |
| | amine_ | | NSLFFMFMLLVGAFT |
| | transferase_ | | NVSHRNVFGYIELIA |
| | CptA | | DVYIICFLLSLCQRT |
| | | | IRQGLVIMLSSVIYV |
| | | | VAIIDTCCKTLFDTP |
| | | | ITPTMLLLAQETTGR |
| | | | EATEFFLQYLNLKLF |
| | | | FSAADIILFLAFCHI |
| | | | VMAVKKMKFSTSYLK |
| | | | QPFVAFVLMFTIFVG |
| | | | MALSIYDKVQLYTVK |
| | | | NLSGLEVAVTNGFAH |
| | | | LYHPVERTVYGLYSN |
| | | | HLIAKQVDGVIMANQ |
| | | | QIKVDSCSFTSPTIV |
| | | | LVIGESANRHHSQLY |
| | | | GYPLPTTPYQLAMKN |
| | | | GKDSLAVFTNVVSPW |
| | | | NLTSKVFKQIFSLQS |
| | | | VDEKGDWSKYVLFPA |
| | | | VFKKAGYHVSFLSNQ |
| | | | FPYGINYTPDWTNNL |
| | | | VGGFFLNHPQLNKQM |
| | | | FDYRNVTIHNYDEDL |
| | | | LNDYKEIISYKKPQL |
| | | | IIFHLLGQHFQYSLR |
| | | | CKSNMKKFGIKDYKR |
| | | | MDLTDKEKQTIADYD |
| | | | NATLYNDFVLNKIVE |
| | | | QFRNKDAIIVYLSDH |
| | | | GEDCYGKDVNMAGRL |
| | | | TEVEQINLKKYHEEF |
| | | | EIPFWIWCSPIYKQR |
| | | | HRKIFTETLMARNNK |
| | | | FMTDDLPHLLLYLAG |
| | | | IKTKDYCEERNVISP |
| | | | SFNNNRRRLVLKTID |
| | | | YDKALYQ |
| 63 | Dipeptide_ | P36837 | MFKNHPKGLLQAAFS |
| | and_ | | NMGERFGYYIMNAVL |
| | tripeptide_ | | ALFLCSKFGLSDETS |
| | permease_B | | GLIASLFLAAIYVMS |
| | | | LVGGVIADRTQNYQR |
| | | | TIESGLVVMALGYVA |
| | | | LSIPVLATPENNSYL |
| | | | LAFTIFALVLIAVGN |
| | | | GLFKGNLQAIVGQMY |
| | | | DDFETEAAKVSPERL |
| | | | KWAQGQRDAGFQIFY |
| | | | VFINLGALAAPFIAP |
| | | | VLRSWWLGRNGLTYD |
| | | | AALPQLCHKYINGTI |
| | | | GDNLGNLQELATKVG |
| | | | GNSADLASFCPHYLD |
| | | | VFNTGVHYSFIASVV |
| | | | TMLISLIIFMSSKKL |
| | | | FPMPGKKEQIVNVEY |
| | | | TDEEKASMAKEIKQR |
| | | | MYALFAVLGISVFFW |
| | | | FSFHQNGQSLSFFAR |
| | | | DFVNTDSVAPEIWQA |
| | | | VNPFFVISLTPLIMW |
| | | | VFAYFTKKGKPISTP |
| | | | RKIAYGMGIAGFAYL |
| | | | FLMGFSLVHNYPSAE |
| | | | QFTSLEPAVRATMKA |
| | | | GPMILILTYFFLTVA |
| | | | ELFISPLGLSFVSKV |
| | | | APKNLQGLCQGLWLG |
| | | | ATAVGNGFLWIGPLM |
| | | | YNKWSIWTCWLVFAI |
| | | | VCFISMVVMFGMVKW |
| | | | LERVTKS |
| 64 | C4- | Q9I4F5 | MQKKIKIGLLPRVII |
| | dicarboxylate_ | | AILLGLFLGYYLPDP |
| | transport_ | | AVRVFLTFNSIFSQF |
| | protein_2 | | LGFMIPLIIIGLVTP |
| | | | AIAGIGKGAGKLLLA |
| | | | TVAIAYVDTIVAGGL |
| | | | SYGTGTWLFPSMIAS |
| | | | TGGAIPHIDKATELT |
| | | | PYFTINIPAMVDVMS |
| | | | SLVFSFIAGLGIAYG |
| | | | GLRTMENL |
| | | | FNEFKTVIEKVIEKA |
| | | | IIPLLPLYIFGVFLS |
| | | | MTHNGQARQVLLVFS |
| | | | QIIIVILVLHVLILI |
| | | | YEFCIAGAIVKHNPF |
| | | | RLLWNMLPAYLTALG |
| | | | TSSSAATIPVTLKQT |
| | | | VKNGVSEEVAGFVVP |
| | | | LCATIHLSGSAMKIT |
| | | | ACALTICMLTDLPHD |
| | | | PGLFIYFILMLAIIM |
| | | | VAAPGVPGGAIMAAL |
| | | | APLSSILGFNEEAQA |
| | | | LMIALYIAMDSFGTA |
| | | | CNVTGDGAIALAVNK |
| | | | FFGKKKETSILS |
| 65 | Inner_ | P76090 | MISVYSIKPQFQRVL |
| | membrane_ | | TPILELLHRAKVTAN |
| | protein_ | | QITLWACVLSLVIGI |
| | YnbA | | LFWFAGDVGTWLYLC |
| | | | LPVGLLIRMALNALD |
| | | | GMMARRYNQITRKGE |
| | | | LLNEVGDVVSDTIIY |
| | | | FPLLKYHPESLYFIV |
| | | | AFIALSIINEYAGVM |
| | | | GKVLSAERRYDGPMG |
| | | | KSDRAFVLGLYGVVC |
| | | | LFGINLSGYSVYIFG |
| | | | VIDLLLVLSTWIRIK |
| | | | KTLKVTRNSQTPE |
| 66 | 2′,3′ | P08331 | MKLSTILLSIMLGLS |
| | cyclic- | | SSTMAQQKDVTIKLI |
| | nucleotide | | ETTDVHGSFFPYDFI |
| | | | TRKPKSGSMARVYTL |
| | | | VEELRKKDGKDNVYL |
| | | | LDNGDILQGQPFSYY |
| | | | YNYVAPEKTNIAASV |
| | | | LNYMGYDVATVGNHD |
| | | | IETGHKVYDKWFKEL |
| | | | KFPILGANIIDTKTN |
| | | | KPYILPYYTIKKKNG |
| | | | IKVCVIGMLTPAIPN |
| | | | WLKESIWSGLRFEEM |
| | | | VSCAKRTMAEVKTQE |
| | | | KPDVIVGLFHSGWDG |
| | | | GIKTPEYDEDASKKV |
| | | | AKEVPGFDIVFFGHD |
| | | | HTPHSSIEKNIVGKD |
| | | | VICLDPANNAQRVAI |
| | | | ATLTLRPKTVKGKRQ |
| | | | YTVTKATGELVDVKE |
| | | | LKADDAFIQHFQPEI |
| | | | DAVKAWSDQVIGRFE |
| | | | NTIYSKDSYFGNSAF |
| | | | NDLILNLELEITKAD |
| | | | IAFNAPLLFNASIKA |
| | | | GPITVADMFNLYKYE |
| | | | NNLCTMRLTGKEIRK |
| | | | HLEMSYDLWCNTMKS |
| | | | PEDHLLLLSSTQNDA |
| | | | QRLGFKNFSFNFDSA |
| | | | AGIDYEVDVTKPDGQ |
| | | | KVRILRMSNGEPFDE |
| | | | NKWYTVAVNSYRANG |
| | | | GGELLTKGAGIPRDS |
| | | | LKSRIIWESPKDQRH |
| | | | YLMEEIKKAGVMNPQ |
| | | | PNHNWKFIPETWTVP |
| | | | AAARDRKLLFGE |
| 67 | Fe(2+)_ | P33650 | MKLSELKTGETGVIV |
| | transporter | | KVSGHGGFRKRIIEM |
| | FcoB | | GFIKGKTVEVLLNAP |
| | | | LQDPVKYKIMGYEVS |
| | | | LRHSEADQIEVLSDV |
| | | | KTHSVGNEEEQEDNQ |
| | | | LEMDSTTYDSTDKEL |
| | | | TPEKQSDAVRRKNHT |
| | | | INVALVGNPNCGKTS |
| | | | LFNFASGAHERVGNY |
| | | | SGVTVDAKVGRAEFD |
| | | | GYVFNLVDLPGTYSL |
| | | | SAYSPEELYVRKQLV |
| | | | DKTPDVVINVIDSSN |
| | | | LERNLYLTTQLIDMH |
| | | | IRMVCALNMFDETEQ |
| | | | RGDHIDAQKLSELFG |
| | | | VPMIPTVFTNGRGVK |
| | | | ELFRQIIAVYEGKED |
| | | | ESLQFRHIHINHGHE |
| | | | I |
| | | | ENGIKEMQEHLKKYP |
| | | | ELCHRYSTRYLAIKL |
| | | | LEHDKDVEQLVSPLG |
| | | | DSIEIFNHRDTAAAR |
| | | | VKEETGNDSETAIMD |
| | | | AKYGFINGALKEANF |
| | | | STGDKKDTYQTTHVI |
| | | | DHVLTNKYFGFPIFF |
| | | | LVLLVMFTATFVIGQ |
| | | | YPMDWIEAGVGWLGE |
| | | | FISKNMPAGPVKDMI |
| | | | VDGIIGGVGAVIVFL |
| | | | PQILILYFFISYMED |
| | | | CGYMSRAAFIMDRLM |
| | | | HKMGLHGKSFIPLIM |
| | | | GFGCNVPAVMATRTI |
| | | | ESRRSRLITMLILPL |
| | | | MSCSARLPIYVMITG |
| | | | SFFALKYRSLAMLSL |
| | | | YIIGVLMAVAMSRLF |
| | | | SAFVVKGEDTPFVME |
| | | | LPPYRFPTWKAIGRH |
| | | | TWEKGKQYLKKMGGI |
| | | | ILVASIIVWALGYFP |
| | | | LPDDPNMDNQARQEQ |
| | | | SYIGRIGKAVEPVFR |
| | | | PQGFNWKLDVGLLSG |
| | | | MGAKEIVASTMGVLY |
| | | | SNDGSFSDDNGYSSE |
| | | | TGKYSKLHNLITKDV |
| | | | ATMHHISYEEAEPIA |
| | | | TLTAFSFLLFVLLYF |
| | | | PCVATIAAIKGETGS |
| | | | WGWALFAAGYTTALA |
| | | | WIVSAVVFQVGMLFM |
| 68 | UDP-N- | P9WJM1 | MESFIIEGGHQLSGT |
| | acetylglucosamine | | IAPQGAKNEALEVIC |
| | | | ATLLTSEEVIIRNVP |
| | | | DILDVNNLIKLLQDI |
| | | | GVKVKKLAPNEFSFQ |
| | | | ADEVNLDYLESSDFV |
| | | | KKCSSLRGSVLMIGP |
| | | | LLGRFGKATIAKPGG |
| | | | DKIGRRRLDTHFLGF |
| | | | KNLGAHFGRVEDRDV |
| | | | YEIQADKLVGTYMLL |
| | | | DEASITGTANIIMAA |
| | | | VLAEGTTTIYNAACE |
| | | | PYIQQLCKMLNAMGA |
| | | | KISGIASNLITIEGV |
| | | | KELHSADHRILPDMI |
| | | | EVGSFIGIAAMIGDG |
| | | | VRIKDVSVPNLGLIL |
| | | | DTFHRLGVQIIVDND |
| | | | DLIIPRQDHYVIDSF |
| | | | IDGTIMTISDAPWPG |
| | | | LTPDLISVLLVVATQ |
| | | | AQGSVLFHQKMFESR |
| | | | LFFVDKLIDMGAQII |
| | | | LCDPHRAVWGHDNAK |
| | | | KLRAGRMSSPDIRAG |
| | | | IALLIAALTAQGTSR |
| | | | IDNIVQIDRGYENIE |
| | | | GRLNALGAKIQRAEV |
| | | | C |
| 69 | Ribitol-5- | Q8RKI9 | MNIAVIFAGGSGLRM |
| | Phosphate_ | | HTKSRPKQFLDLNGK |
| | cytidylyl | | PIIIYTLELFDNHPN |
| | transferase | | IDAIVVACIESWIPF |
| | | | LEKQLRKFEINKVVK |
| | | | IIPGGKSGQESIYKG |
| | | | LCAAEEYAQSKGVSN |
| | | | EETTVLIHDGVRPLI |
| | | | TEETITDNIKKVEEV |
| | | | GSCITCIPATETLIV |
| | | | KQADDALEIPSRADS |
| | | | FIARAPQSFRLIDII |
| | | | TAHRRSLAEGKADFI |
| | | | DSCTMMSHYGYKLGT |
| | | | IIGPMENIKITTPTD |
| | | | FFVLRAMVKVHEDQQ |
| | | | IFGL |

In some embodiments, the hemoglobin-dependent Prevotella strain is a strain of Prevotella bacteria comprising one or more of the proteins listed in Table 1 and that is free or substantially free of one or more proteins listed in Table 2. In some embodiments, the hemoglobin-dependent Prevotella strain is a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

### Hemoglobin Substitutes

As disclosed herein, certain algae, algae biomasses and algae-derived components are able to be used in culture media in place of hemoglobin to facilitate the growth of otherwise hemoglobin-dependent bacteria.

The hemoglobin substitutes provided herein support the growth of hemoglobin-dependent bacteria in the absence or hemoglobin or a derivative thereof. The hemoglobin substitutes provided herein also can support the growth of hemoglobin-dependent bacteria with use of reduced amounts of hemoglobin or a derivative thereof. For example, the culture contains a lower amount of hemoglobin (e.g., less than about 0.02 g/L hemoglobin; e.g., about 0.01 g/L or about 0.005 g/L or less hemoglobin) in combination with a hemoglobin substitute described herein, yet comparable growth of the hemoglobin-dependent bacteria is achieved compared to growth of the same bacteria in media containing typical amounts of hemoglobin.

In some embodiments, the hemoglobin substitute used in the methods and compositions provided herein is spirulina or components thereof (i.e., spirulina components able to substitute for hemoglobin to support growth of otherwise hemoglobin-dependent bacteria, such as a soluble spirulina component). As disclosed herein, spirulina components are capable of facilitating growth of hemoglobin-dependent bacteria following filtration, indicating that soluble components of spirulina are hemoglobin substitutes.

In some embodiments, the hemoglobin substitute used in the methods and compositions provided herein is a cyanobacteria, a cyanobacteria biomass and/or a cyanobacteria component (i.e., a cyanobacteria, cyanobacteria biomass and/or cyanobacteria component able to substitute for hemoglobin to support growth of otherwise hemoglobin-dependent bacteria). In certain embodiments, any cyanobacteria, cyanobacteria biomass, or cyanobacteria component that is capable of functioning as a hemoglobin substitute can be used in the methods and compositions provided herein. In certain embodiments, the cyanobacteria is of the order Oscillatoriales. In some embodiments, the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema. In some embodiments, the cyanobacteria is Arthrospira platensis and/or Arthrospira maxima.

In some embodiments, the hemoglobin substitute used in the methods and compositions provided herein is a green algae, a green algae biomass and/or a green algae component (i.e., a green algae, green algae biomass and/or green algae component able to substitute for hemoglobin to support growth of otherwise hemoglobin-dependent bacteria). In certain embodiments, any green algae, green algae biomass, or a green algae component that is capable of functioning as a hemoglobin substitute can be used in the methods and compositions provided herein. In certain embodiments, the green algae is of the order Chlorellales. In some embodiments, the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

In some embodiments, the hemoglobin substitute is sterilized, e.g., prior to combining with other components of a growth media. Sterilization may be by Ultra High Temperature (UHT) processing, autoclaving or filtering. In some embodiments, the hemoglobin substitute is autoclaved. In some embodiments, the hemoglobin substitute is filtered.

### Growth Media

In some embodiments, provided herein is growth media comprising a hemoglobin substitute disclosed herein. In certain embodiments, the growth media comprises an amount of a hemoglobin substitute disclosed herein (e.g., spirulina or a component thereof (e.g., a soluble component)) sufficient to support growth of hemoglobin-dependent bacteria. In certain embodiments, the growth media comprises at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a hemoglobin substitute disclosed herein (e.g., spirulina or a component thereof). In some embodiments, the growth medium comprises about 1 g/L of a hemoglobin substitute disclosed herein. In some embodiments, the growth medium comprises about 2 g/L of a hemoglobin substitute disclosed herein. In some embodiments, the growth media provided herein comprises at least 1 g/L and no more than 3 g/L of a hemoglobin substitute disclosed herein (e.g., spirulina or a component thereof). In some embodiments, the growth media comprises at least 1 g/L and no more than 2 g/L of a hemoglobin substitute disclosed herein (e.g., spirulina or a component thereof). In some embodiments of the methods and compositions provided herein, the growth media does not comprise hemoglobin or a derivative thereof. In some embodiments, the growth media does not comprise animal products.

In some embodiments, the growth media contains a component of spirulina, cyanobacteria or green algae, such as a soluble component of spirulina, a cyanobacteria or a green algae disclosed herein. In some embodiments, the growth media contains a soluble component of spirulina, a cyanobacteria or a green algae disclosed herein. For example, a supernatant obtained from a spirulina solution (e.g., a resuspended spirulina solution (e.g., a liquid mixture from lyophilized biomass) can be used in the growth media (e.g., the supernatant is obtained after the spirulina solution is filtered or centrifuged)).

In some embodiments the growth media may contain sugar, yeast extracts, plant based peptones, buffers, salts, trace elements, surfactants, anti-foaming agents, and/or vitamins.

In some embodiments, the growth media comprise yeast extract, soy peptone A2SC 19649, Soy peptone E110 19885, dipotassium phosphate, monopotassium phosphate, L-cysteine-HCl, ammonium chloride, glucidex 21 D, and/or glucose.

In some embodiments, the growth media comprises 5 g/L to 15 g/L yeast extract 19512. In some embodiments, the growth media comprises 10 g/L yeast extract 19512.

In some embodiments, the growth media comprises 10 g/L to 15 g/L soy peptone A2SC 19649. In some embodiments, the growth media comprises 12.5 g/L soy peptone A2SC 19649. In some embodiments, the growth media comprises 10 g/L soy peptone A2SC 19649.

In some embodiments, the growth media comprises 10 g/L to 15 g/L Soy peptone E110 19885. In some embodiments, the growth media comprises 12.5 g/L Soy peptone E110 19885. In some embodiments, the growth media comprises 10 g/L soy peptone E110 19885.

In some embodiments, the growth media comprises 1 g/L to 3 g/L dipotassium phosphate. In some embodiments, the growth media comprises 1.59 g/L dipotassium phosphate. In some embodiments, the growth media comprises 2.5 g/L dipotassium phosphate.

In some embodiments, the growth media comprises 0 g/L to 1.5 g/L monopotassium phosphate. In some embodiments, the growth media comprises 0.91 g/L monopotassium phosphate. In some embodiments, the growth media does not comprise monopotassium phosphate.

In some embodiments, the growth media comprises 0.1 g/L to 1.0 g/L L-cysteine-HCl. In some embodiments, the growth media comprises 0.5 g/L L-cysteine-HCl.

In some embodiments, the growth media comprises 0 g/L to 1.0 g/L ammonium chloride. In some embodiments, the growth media comprises 0.5 g/L ammonium chloride. In some embodiments, the growth media does not comprise ammonium chloride.

In some embodiments, the growth media comprises 0 g/L to 30 g/L glucidex 21 D. In some embodiments, the growth media comprises 25 g/L glucidex 21 D. In some embodiments, the growth media does not comprise glucidex 21 D.

In some embodiments, the growth media comprises 5 g/L to 15 g/L glucose. In some embodiments, the growth media comprises 10 g/L glucose. In some embodiments, the growth media comprises 5 g/L glucose.

In some embodiments, the growth media comprises 5 g/L to 15 g/L N-acetyl-glucosamine (NAG). In some embodiments, the growth media comprises 10 g/L NAG. In some embodiments, the growth media comprises 5 g/L NAG.

In certain embodiments, the growth media comprises a hemoglobin substitute provided herein, about 10 g/L yeast extract 19512, about 12.5 g/L soy peptone A2SC 19649, about 12.5 g/L soy peptone E110 19885, about 1.59 g/L dipotassium phosphate, about 0.91 g/L monopotassium phosphate, about 0.5 g/L ammonium chloride, about 25 g/L glucidex 21 D, and/or about 10 g/L glucose. In some embodiments, the growth medium is the growth medium of Table 3.

In certain embodiments, the growth media comprises a hemoglobin substitute provided herein, about 10 g/L yeast extract 19512, about 10 g/L soy peptone A2SC 19649, about 10 g/L soy peptone E110 19885, about 2.5 g/L dipotassium phosphate, about 0.5 g/L L-cysteine-HCl, and/or about 5 g/L glucose. In some embodiments, the growth medium is the growth medium of Table 4.

In certain embodiments, the growth media is at a pH of 5.5 to 7.5. In some embodiments, the growth media is at a pH of about 6.5.

In some embodiments, prior to being added to the growth media, cyanobacteria, or a biomass thereof, e.g., spirulina is prepared as a liquid mixture from lyophilized biomass and sterilized by autoclaving or filtration. In some embodiments, the lyophilized biomass of spirulina is added to the growth media, which is then sterilized as described below.

In some embodiments, the media is sterilized. Sterilization may be by Ultra High Temperature (UHT) processing, autoclaving or filtering. The UHT processing is performed at very high temperature for short periods of time. The UHT range may be from 135-180° C. For example, the medium may be sterilized from between 10 to 30 seconds at 135° C.

### Culturing Methods

In certain aspects, provided herein are methods and/or compositions that facilitate the growth of hemoglobin-dependent bacteria. Such methods may comprise incubating the hemoglobin-dependent bacteria in a growth media provided herein. The methods may comprise maintaining the temperature and pH of the growth media as disclosed herein. The culturing may begin in a relatively small volume of growth media (e.g., 1 L) where bacteria are allowed to reach the log phase of growth. Such culture may be transferred to a larger volume of growth media (e.g., 20 L) for further growth to reach a larger biomass. Depending on the need of the final amount of biomass, such transfer may be repeated more than once. The methods may comprise the incubation of the hemoglobin-dependent bacteria in bioreactors.

In certain aspects, the hemoglobin-dependent bacteria are incubated at a temperature of 35° C. to 39° C. In some embodiments, the hemoglobin-dependent bacteria are incubated at a temperature of about 37° C.

In certain embodiments, the methods and/or compositions provided herein increase the growth rate of hemoglobin-dependent bacteria such that hemoglobin-dependent bacteria grow at an increased rate in the growth media comprising a hemoglobin substitute disclosed herein (e.g., spirulina or a component thereof), compared to the rate at which the hemoglobin-dependent bacteria grow in the same growth media but without the hemoglobin substitute disclosed herein. In some embodiments, the rate at which the hemoglobin-dependent bacteria grow in the growth media comprising a hemoglobin substitute disclosed herein (e.g., spirulina or a component thereof) is higher than the rate at which the hemoglobin-dependent bacteria grow in the same growth media but without the hemoglobin substitute disclosed herein by at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400%. In some embodiments, the growth rate is increased by about 200% to about 400%. The rate may be measured as the cell density (as measured by e.g., optical density at the wavelength of 600 nm (OD600)) reached within a given amount of time. In certain embodiments, such rate is measured and compared during the log phase (or exponential phase) of the bacterial growth, optionally wherein the log phase is early log phase.

In certain embodiments, the methods and/or compositions provided herein increase the bacterial cell density such that the hemoglobin-dependent bacteria grow to a higher bacterial cell density in the growth media comprising a hemoglobin substitute disclosed herein (e.g., spirulina or a component thereof), compared to the cell density to which the hemoglobin-dependent bacteria grow in the same growth media but without the hemoglobin substitute disclosed herein. In some embodiments, the hemoglobin-dependent bacteria grow to a cell density in the growth media comprising a hemoglobin substitute disclosed herein (e.g., spirulina or a component thereof) is higher than the cell density to which the hemoglobin-dependent bacteria grow in the same growth media but without the hemoglobin substitute disclosed herein by at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400%. In some embodiments, the bacterial cell density higher than about 200% to about 400%. The cell density may be measured (e.g., by OD600 or by cell counting) at the stationary phase of bacterial growth, optionally wherein the stationary phase is early stationary phase. In some embodiments, the stationary phase is determined as the phase where the growth rate is retarded followed by an exponential phase of growth (e.g., from a growth curve). In other embodiments, the stationary phase is determined by the low glucose level in the growth media.

In some embodiments, the methods provided herein comprise incubating the hemoglobin-dependent bacteria under anaerobic atmosphere. In certain aspects, provided herein are methods of culturing hemoglobin-dependent bacteria under anaerobic atmosphere comprising CO₂. In some embodiments, the anaerobic atmosphere comprises greater than 1% CO₂. In some embodiments, the anaerobic atmosphere comprises greater than 5% CO₂. In some embodiments, the anaerobic atmosphere comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic atmosphere comprises at least 10% CO₂. In some embodiments, the anaerobic atmosphere comprises at least 20% CO₂. In some embodiments, the anaerobic atmosphere comprises from 10% to 40% CO₂. In some embodiments, the anaerobic atmosphere comprises from 20% to 30% CO₂. In some embodiments, the anaerobic atmosphere comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 3′7%, about 38%, about 39%, or about 40% CO₂. In some embodiments, the anaerobic atmosphere comprises about 25% CO₂.

In certain aspects, the anaerobic atmosphere comprises N₂. In some embodiments, the anaerobic atmosphere comprises less than 95% N₂. In some embodiments, the anaerobic atmosphere comprises less than 90% N₂. In some embodiments, the anaerobic atmosphere comprises less than 95%, less than 92%, less than 90%, less than 87%, less than 85%, less than 82%, less than 80%, less than 77% N₂. In some embodiments, the anaerobic atmosphere comprises less than 85% N₂. In some embodiments, the anaerobic atmosphere comprises less than 80% N₂. In some embodiments, the anaerobic atmosphere comprises from 65% to 85% N₂. In some embodiments, the anaerobic atmosphere comprises from 70% to 80% N₂. In some embodiments, the anaerobic atmosphere comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic atmosphere comprises about 75% N₂.

In some embodiments, the anaerobic atmosphere consists essentially of CO₂ and N₂. In some embodiments, the anaerobic atmosphere comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

Thus, in some embodiments provided herein are methods of culturing hemoglobin-dependent bacteria under anaerobic conditions comprising a greater level of CO₂ compared to conventional anaerobic culture conditions (e.g., at a level of greater than 1% CO₂, e.g., at a level of greater than 5% CO₂, such as at a level of about 25% CO₂). In certain embodiments, provided herein are bioreactors comprising hemoglobin-dependent bacteria being cultured under conditions comprising a greater level of CO₂ compared to conventional anaerobic culture conditions (e.g., at a level of greater than 1% CO₂, such as at a level of about 25% CO₂). In some embodiments, the methods and compositions provided herein result in increased bacterial yield compared to conventional culture conditions.

In certain aspects, provided herein are methods of culturing hemoglobin-dependent bacteria under anaerobic conditions comprising a lower level of N₂ compared to conventional anaerobic culture conditions (e.g., at a level of less than 95% N₂, e.g., at a level of less than 90% N₂, such as at a level of about 75% N₂). In certain embodiments, provided herein are bioreactors comprising hemoglobin-dependent bacteria being cultured under conditions comprising a lower level of N₂ compared to conventional anaerobic culture conditions (e.g., at a level of less than 95% N₂ such as at a level of about 75% N₂). In some embodiments, the methods and compositions provided herein result in increased bacterial yield compared to conventional culture conditions.

In certain aspects, provided herein are methods of culturing hemoglobin-dependent bacteria, the method comprises the steps of a) purging a bioreactor with an anaerobic gaseous mixture comprising greater than 1% CO₂, and b) culturing the hemoglobin-dependent bacteria in the bioreactor purged in step a). In some embodiments, the anaerobic gaseous mixture comprises greater than 1% CO₂. In some embodiments, the anaerobic gaseous mixture comprises at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% CO₂. In some embodiments, the anaerobic gaseous mixture comprises at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CO₂. In some embodiments, the anaerobic gaseous mixture comprises from 5% to 35% CO₂, 10% to 40% CO₂, 10% to 30% CO₂, 15% to 30% CO₂, 20% to 30% CO₂, 22% to 28% CO₂, or 24%, to 26% CO₂. In some embodiments, the anaerobic gaseous mixture comprises greater than 5% CO₂. In some embodiments, the anaerobic gaseous mixture comprises at least 10% CO₂. In some embodiments, the anaerobic gaseous mixture comprises at least 20% CO₂. In some embodiments, the anaerobic gaseous mixture comprises from 10% to 40% CO₂. In some embodiments, the anaerobic gaseous mixture comprises from 20% to 30% CO₂. In some embodiments, the anaerobic gaseous mixture comprises about 25% CO₂.

In certain aspects, provided herein are methods of culturing hemoglobin-dependent bacteria, the method comprises the steps of a) purging a bioreactor with an anaerobic gaseous mixture comprising less than 95% N₂; and b) culturing the hemoglobin-dependent bacteria in the bioreactor purged in step a). In some embodiments, the anaerobic gaseous mixture comprises less than 95% N₂. In some embodiments, the anaerobic gaseous mixture comprises less than 95%, less than 92%, less than 90%, less than 87%, less than 85%, less than 82%, less than 80%, less than 77% N₂. In some embodiments, the anaerobic gaseous mixture comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic gaseous mixture comprises less than 95% N₂. In some embodiments, the anaerobic gaseous mixture comprises less than 90% N₂. In some embodiments, the anaerobic gaseous mixture comprises from 65% to 85% N₂. In some embodiments, the anaerobic gaseous mixture comprises from 70% to 80% N₂CO₂. In some embodiments, the anaerobic gaseous mixture comprises about 75% N₂.

In some embodiments, the anaerobic gaseous mixture consists essentially of CO₂ and N₂. In some embodiments, the anaerobic gaseous mixture comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In some embodiments, the anaerobic gaseous mixture comprises CO₂ and N₂ in a ratio of about 1:99, about 2:98, about 3:97, about 4:96, about 5:95, about 6:94, about 7:93, about 8:92, about 9:91, about 10:90, 11:89, about 12:88, about 13:87, about 14:86, about 15:85, about 16:84, about 17:83, about 18:82, about 19:81, about 20:80, 21:79, about 22:78, about 23:77, about 24:76, about 25:75, about 26:74, about 27:73, about 28:72, about 29:71, about 30:70, 31:69, about 32:68, about 33:67, about 34:66, about 35:65, about 36:64, about 37:63, about 38:62, about 39:61, or about 40:50 CO₂ to N₂. In some embodiments, the mixed gas composition provides an atmosphere in the bioreactor comprising CO₂ and N₂ in a ratio of about 25:75.

In some embodiments, an anaerobic gaseous mixture is continuously added to the bioreactor during culturing. In some embodiments, the continuously added anaerobic gaseous mixture is added at a rate of 0.01 to 0.1 vvm. In some embodiments the continuously added anaerobic gaseous mixture is added at a rate of 0.02 vvm. In some embodiments, the continuously added anaerobic gaseous mixture comprises any one of gaseous mixtures described above.

In some embodiments, the methods provided herein further comprises the step of inoculating a growth media with the hemoglobin-dependent bacteria, wherein the bacteria are cultured in the growth media according to the methods provided herein. In some embodiments, the volume of the inoculated hemoglobin-dependent bacteria is between 0.01% and 10% v/v of the growth media (e.g., about 0.1% v/v of the growth media, about 0.5% v/v of the growth media, about 1% v/v of the growth media, about 5% v/v of the growth media). In some embodiments, the volume of hemoglobin-dependent bacteria is about 1 mL.

In some embodiments, inoculum can be prepared in flasks or in smaller bioreactors where growth is monitored. For example, the inoculum size may be between approximately 0.1% v/v and 5% v/v of the total bioreactor volume. In some embodiments, the inoculum is 0.1-3% v/v, 0.1-1% v/v, 0.1-0.5% v/v, or 0.5-1% v/v of the total bioreactor volume. In some embodiments, the inoculum is about 0.1% v/v, about 0.2% v/v, about 0.3% v/v, about 0.4%, v/v, about 0.5% v/v, about 0.6% v/v, about 0.7% v/v, about 0.8% v/v, about 0.9% v/v, about 1% v/v, about 1.5% v/v, about 2% v/v, about 2.5% v/v, about 3% v/v, about 4%, v/v, or about 5% v/v of the total bioreactor volume.

In some embodiments, before the inoculation, the bioreactor is prepared with growth medium at desired pH and temperature. The initial pH of the culture medium may be different than the process set-point. pH stress may be detrimental at low cell concentration; the initial pH could be between pH 7.5 and the process set-point. For example, pH may be set between 4.5 and 8.0, preferably 6.5. During the fermentation, the pH can be controlled through the use of sodium hydroxide, potassium hydroxide, or ammonium hydroxide. The temperature may be controlled from 25° C. to 45° C., for example at 37° C.

In some embodiments, depending on strain and inoculum size, the bioreactor fermentation time can vary. For example, fermentation time can vary from 5 hours to 48 hours. In some embodiments, fermentation time may be from 5 hours to 24 hours, 8 hours to 24 hours, 8 hours to 18 hours, 8 hours to 16 hours, 8 hours to 14 hours, 10 hours to 24 hours, 10 hours to 18 hours, 10 hours to 16 hours, 10 hours to 14 hours, 10 hours to 12 hours, 12 hours to 24 hours, 12 hours to 18 hours, 12 hours to 16 hours, or 12 hours to 14 hours.

In some embodiments, culturing the hemoglobin-dependent bacteria comprises agitating the culture at a RPM of 50 to 300. In some embodiments, the hemoglobin-dependent bacteria is agitated at a RPM of about 150.

For example, in some embodiments, a culturing method comprises culturing the hemoglobin-dependent bacteria for at least 5 hours (e.g., at least 10 hours). In some embodiments, the hemoglobin-dependent bacteria is cultured for 10-24 hours. In some embodiments, the hemoglobin-dependent bacteria is cultured for 14 to 16 hours. In some embodiments, the method further comprises the step of inoculating about 5% v/v of the cultured bacteria in a growth media. In some embodiments, the growth media is about 20 L in volume. In some embodiments, the hemoglobin-dependent bacteria is cultured for 10-24 hours. In some embodiments, the hemoglobin-dependent bacteria is cultured for 12-14 hours. In some embodiments, the method further comprises the step of inoculating about 0.5% v/v of the cultured bacteria in a growth medium. In some embodiments, the growth medium is about 3500 L in volume. In some embodiments, the hemoglobin-dependent bacteria is cultured for 10-24 hours. In some embodiments, the hemoglobin-dependent bacteria is cultured for 12-14 hours. In some embodiments, the hemoglobin-dependent bacteria is cultured at least until a stationary phase is reached.

In certain embodiments, the culturing method further comprises the step of harvesting the cultured bacteria. The harvest time may be based on either glucose level is below 2 g/L or when stationary phase is reached. In some embodiments, the method further comprises the step of centrifuging the cultured bacteria after harvesting (e.g., to produce a cell paste). In some embodiments, the method further comprises diluting the cell paste with a stabilizer solution to produce a cell slurry. In some embodiments, the method further comprises the step of lyophilizing the cell slurry to produce a powder. In some embodiments, the method further comprises irradiating the powder with gamma radiation.

For example, in some embodiments, once fermentation complete, the culture is cooled (e.g., to 10° C.) and centrifuged collecting the cell paste. A stabilizer may be added to the cell paste and mixed thoroughly. Harvesting may be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets may be mixed with excipients and submerged in liquid nitrogen.

In certain embodiments, the cell slurry may be lyophilized. Lyophilization of material, including live bacteria, may begin with primary drying. During the primary drying phase, the ice is removed. Here, a vacuum is generated and an appropriate amount of heat is supplied to the material for the ice to sublime. During the secondary drying phase, product bound water molecules may be removed. Here, the temperature is raised higher than in the primary drying phase to break any physico-chemical interactions that have formed between the water molecules and the product material. The pressure may also be lowered further to enhance desorption during this stage. After the freeze-drying process is complete, the chamber may be filled with an inert gas, such as nitrogen. The product may be sealed within the freeze dryer under dry conditions, preventing exposure to atmospheric water and contaminants. The lyophilized material may be gamma irradiated (e.g., 17.5 kGy).

### Bioreactors

In certain aspects, provided herein are bioreactors comprising growth media provided herein (i.e., a growth media comprising a hemoglobin substitute disclosed herein (e.g., spirulina or a component thereof)) and/or hemoglobin-dependent bacteria provided herein. In some embodiments, the hemoglobin-dependent bacteria are Prevotella bacteria (e.g., a Prevotella strain provided herein). In some embodiments, provided herein are methods of culturing bacteria in such bioreactors.

In certain embodiments, the bioreactor is under the anaerobic conditions mentioned above. In certain aspects, provided herein are bioreactors comprising hemoglobin-dependent bacteria under an anaerobic atmosphere disclosed above. In certain aspects, provided herein are bioreactors of various sizes. In some embodiments, the bioreactors are at least 1 L in volume, at least 5 L in volume, at least 10 L in volume, at least 15 L in volume, at least 20 L in volume, at least 30 L in volume, at least 40 L in volume, at least 50 L in volume, at least 100 L in volume, at least 200 L in volume, at least 250 L in volume, at least 500 L in volume, at least 750 L in volume, at least 1000 L in volume, at least 1500 L in volume, at least 2000 L in volume, at least 2500 L in volume, at least 3000 L in volume, at least 3500 L in volume, at least 4000 L in volume, at least 5000 L in volume, at least 7500 L in volume, at least 10,000 L in volume, at least 15,000 L in volume, or at least 20,000 L in volume. In some embodiments, the bioreactors are about 1 L in volume, about 5 L in volume, about 10 L in volume, about 15 L in volume, about 20 L in volume, about 30 L in volume, about 40 L in volume, about 50 L in volume, about 100 L in volume, about 200 L in volume, about 250 L in volume, about 500 L in volume, about 750 L in volume, about 1000 L in volume, about 1500 L in volume, about 2000 L in volume, about 2500 L in volume, about 3000 L in volume, about 3500 L in volume, about 4000 L in volume, about 5000 L in volume, about 7500 L in volume, about 10,000 L in volume, about 15,000 L in volume, or about 20,000 L in volume.

### EXAMPLES

### Example 1: Materials and Methods

### Preparation of Growth Media

A hemoglobin solution was prepared by dissolving the porcine hemoglobin in 0.01 M NaOH. The solution was sterilized by autoclaving. A working concentration of 20 mg/L or 200 mg/L was used.

Spirulina was prepared by powdering the spirulina tablets and dissolving the powder in water or 0.01 M NaOH. The solution was sterilized by autoclaving, and was added to the growth media at various working concentrations (e.g., 0.02 g/L, 0.2 g/L, or 2 g/L).

Chlorophyllin (Sigma cat #11006-34-1) was dissolved in water or 0.01 M NaOH and autoclaved before adding to the growth media at a final concentration of 0.02 g/L, 0.05 g/L, 0.1 g/L, or 0.2 g/L.

Vitamin B12 and FeCl2 were tested as growth supplements either alone or in combination. Vitamin B12 solution was prepared by dissolving in water and filter sterilizing using a 0.22 μm filter.

### Growth Analysis

Four replicates were performed for each growth analysis. 0.1% inoculum from a frozen cell bank was used for each culture. Bacteria were grown in the SPYG1 media as described below. Kinetics of bacterial growth were measured by measuring the optical density (OD600) every 30 minutes on a plate reader for 48 hours while culturing in the anaerobic environment at 37° C.

### Example 2: Exemplary Manufacturing Process of Hemoglobin-dependent Bacteria

An exemplary manufacturing process of hemoglobin-dependent bacteria, e.g., Prevotella histicola is presented herein. In this exemplary method the hemoglobin-dependent bacteria are grown in growth media comprising the components listed in Table 4. The media is filter sterilized prior to use.

TABLE 3
Exemplary Growth Media

| Component | g/L |
|---|---|
| Yeast Extract 19512 | 10 |
| Soy Peptone A2SC 19649 | 12.5 |
| Soy Peptone E110 19885 | 12.5 |
| Dipotassium Phosphate K2HPO4 | 1.59 |
| Monopotassium phosphate | 0.91 |
| L-Cysteine-HCl | 0.5 |
| Ammonium chloride | 0.5 |
| Glucidex 21 D (Maltodextrin) | 25 |
| Glucose | 10 |
| Spirulina | 1 |

TABLE 4
Another Exemplary Growth Media (SPYG1 media)

| Component | g/L |
|---|---|
| Yeast Extract 19512 Organotechnie S.A.S. | 10 |
| Soy Peptone A2SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |
| Glucose | 5 |
| Spirulina | 1 |

Briefly, a 1 L bottle is inoculated with a 1 mL of a cell bank sample that had been stored at −80° C. This inoculated culture is incubated in an anaerobic chamber at 37° C., pH=6.5 due to sensitivity of this strain to aerobic conditions. When the bottle reaches log growth phase (after approximately 14 to 16 hours of growth), the culture is used to inoculate a 20 L bioreactor at 5% v/v. During log growth phase (after approximately 10 to 12 hours of growth), the culture is used to inoculate a 3500 L bioreactor at 0.5% v/v.

Fermentation culture is continuously mixed with addition of a mixed gas at 0.02 VVM with a composition of 25% CO₂ and 75% N₂. pH is maintained at 6.5 with ammonium hydroxide and temperature controlled at 37° C. Harvest time is based on when stationary phase is reached (after approximately 12 to 14 hours of growth).

Once fermentation complete, the culture is cooled to 10° C., centrifuged and the resulting cell paste is collected. 10% Stabilizer is added to the cell paste and mixed thoroughly (Stabilizer Concentration (in slurry): 1.5% Sucrose, 1.5% Dextran, 0.03% Cysteine). The cell slurry is lyophilized and gamma irradiated (17.5 kGy at room temperature).

For other growth conditions that can be used, see, e.g., WO 2019/051381, the disclosure of which is hereby incorporated by reference.

TABLE 5
Stabilizer Formulation

| Component | g/kg |
|---|---|
| Sucrose | 200 |
| Dextran 40k | 200 |
| Cysteine HCl | 4 |
| Water | 596 |

### Example 3: Vitamin B12 and/or FeCl2 Cannot Facilitate Growth of Hemoglobin-Dependent Bacteria in the Absence of Hemoglobin

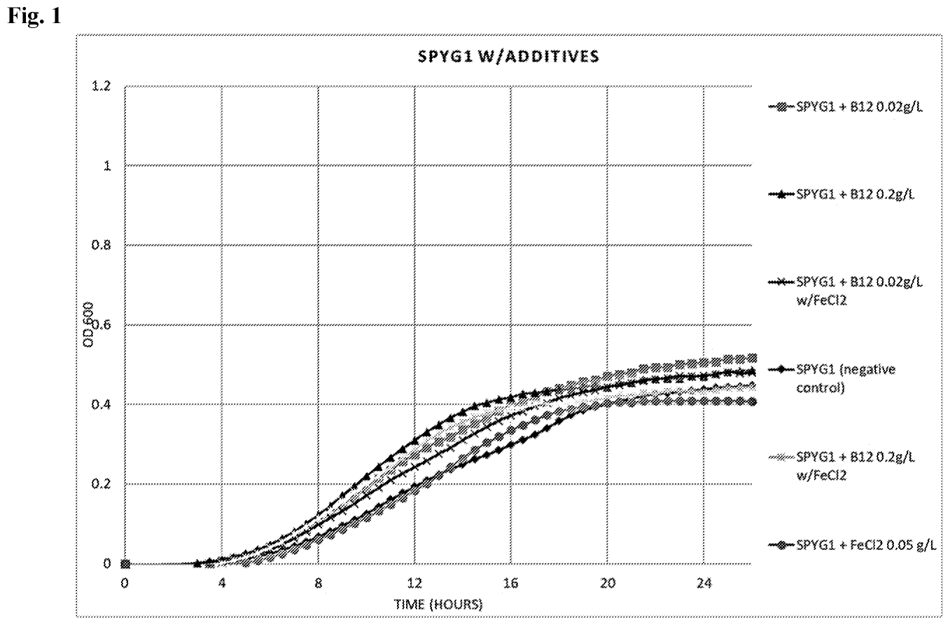

In order to find an alternative source of a GMP-grade supplement for growing hemoglobin-dependent bacteria, non-animal products such as vitamin B12 and/or FeCl2 were tested as growth supplements. Representative hemoglobin-dependent bacteria, Prevotella Strain B 50329 (NRRL accession number B 50329), were grown as described in Example 1 in the SPYG1 media supplemented with vitamin B12 and/or FeCl2. Various amounts of vitamin B12, FeCl2 (the hemoglobin-associated iron), or a combination thereof in growth media did not improved the growth of hemoglobin-dependent bacteria, compared to growth media without any supplement. As seen in FIG. 1, vitamin B12 and FeCl2 cannot substitute for hemoglobin to facilitate the growth of hemoglobin-dependent bacteria.

### Example 4: Spirulina Can Substitute for Hemoglobin to Facilitate the Growth of Hemoglobin-Dependent Bacteria

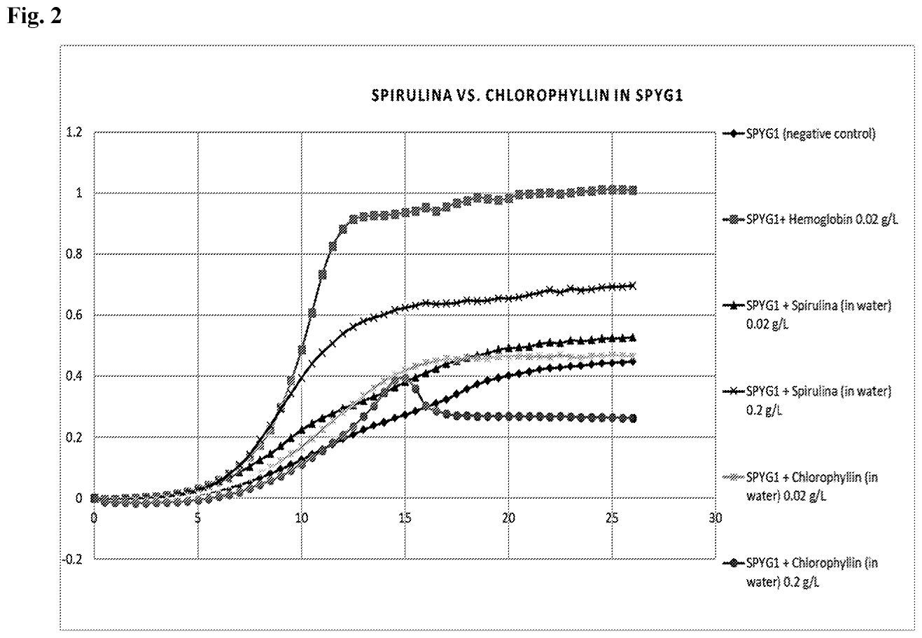

In contrast to vitamin B12 or FeCl2, addition of spirulina to growth media improved the growth of hemoglobin-dependent bacteria (Prevotella Strain B 50329 (NRRL accession number B 50329)) in the absence of hemoglobin. Addition of 0.2 g/L spirulina enhanced the growth of bacteria and led to an increase in both growth rate and the cell density (FIG. 2). Thus, spirulina promotes growth of hemoglobin-dependent bacteria in a dose-dependent manner in the absence of hemoglobin, as 0.2 g/L of spirulina enhanced growth as compared to 0.02 g/L spirulina.

In order to determine whether chlorophyllin can improve the growth of hemoglobin-dependent bacteria in the absence of hemoglobin, various amounts of chlorophyllin was titrated into the growth media. Rather than improving growth, chlorophyllin at a concentration of 0.2 g/L inhibited the growth of hemoglobin-dependent bacteria (FIG. 2). Even at a lower concentration of 0.02 g/L, chlorophyllin did not improve the growth of hemoglobin-dependent bacteria (FIG. 2).

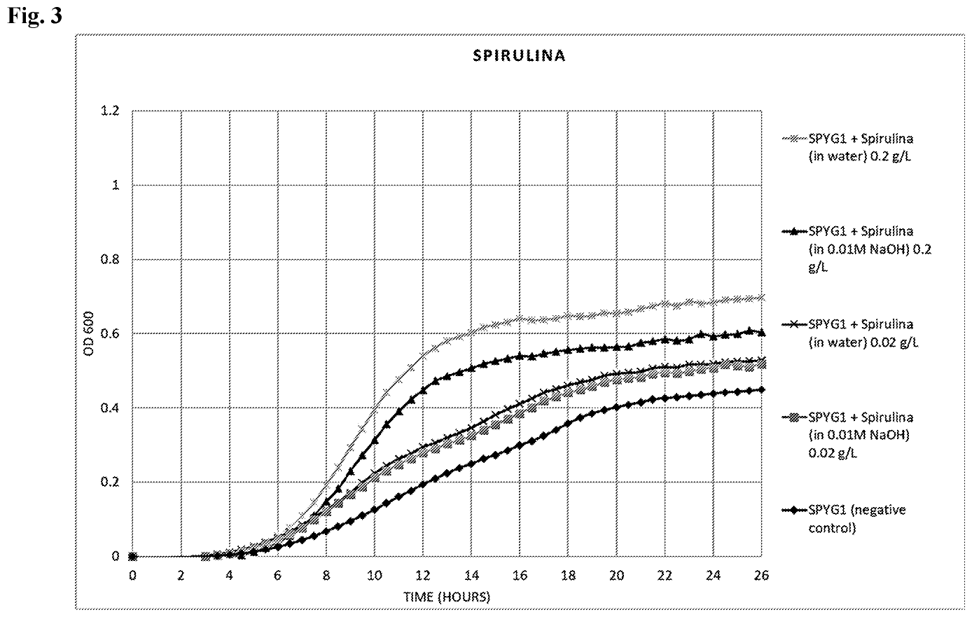

To determine the optimal solvent for dissolving spirulina, the ability of spirulina dissolved in water vs. 0.01 M NaOH to support the growth of hemoglobin-dependent bacteria in the absence of hemoglobin was compared. Hemoglobin-dependent bacteria grew at a faster rate and to a higher cell density when grown in media comprising spirulina dissolved in water compared to spirulina dissolved in 0.01 M NaOH (FIG. 3) although spirulina in both water and NaOH supported growth of hemoglobin-dependent bacteria in the absence of hemoglobin and to a greater extent than the negative control.

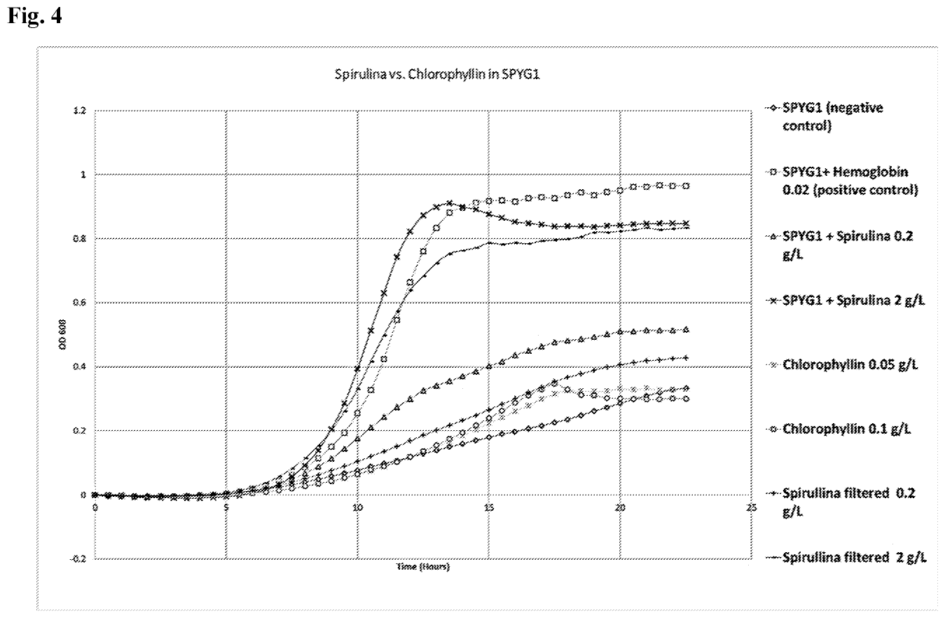

In order to determine whether spirulina can substitute for hemoglobin or a derivative thereof, hemoglobin-dependent bacteria (Prevotella histicola) were cultured in growth media comprising various amounts of spirulina and their growth curves were compared with those of bacteria cultured in media supplemented with hemoglobin or chlorophyllin. At 2 g/L, spirulina supported the growth of hemoglobin-dependent bacteria comparably to hemoglobin (FIG. 4). In fact, bacteria cultured in growth media comprising 2 g/L of spirulina showed faster growth rate compared to the media comprising hemoglobin (FIG. 4). As seen in FIG. 2, chlorophyllin did not support the growth of hemoglobin-dependent bacteria at any concentration tested (FIG. 4). Spirulina solution sterilized by filtration was also effective in supporting the growth of bacteria, indicating that it is compatible with different modes of sterilization, including autoclaving and filtration, and the soluble components of spirulina are sufficient to support growth of the hemoglobin-dependent bacteria.

### Example 5: Hemoglobin-Dependent Bacteria Cultured in Growth Media Comprising Spirulina Are Efficacious in a Mouse Model of Delayed-Type Hypersensitivity (DTH)

Spirulina (in the absence of hemoglobin) facilitates the production of hemoglobin-dependent bacteria that are functionally equivalent to the hemoglobin-dependent bacteria cultured in the presence of hemoglobin. To test whether spirulina facilitates the production of hemoglobin-dependent bacteria that are functionally equivalent to the hemoglobin-dependent bacteria cultured in the presence of hemoglobin, hemoglobin-dependent bacteria cultured in the presence of spirulina or hemoglobin were compared for their efficacy in a mouse model of delayed-type hypersensitivity (DTH).

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006. 99(2): 104-115; see also Irving C. Allen (ed.) Mouse Models of Innate Immunity: Methods and Protocols, Methods in Molecular Biology, 2013. vol. 1031, DOI 10.1007/978-1-62703-481-4_13). It can be induced in a variety of mouse and rat strains using various antigens, for example an antigen emulsified with Complete Freund's Adjuvant, (CFA) or other adjuvant. DTH is characterized by sensitization as well as an antigen-specific T cell-mediated reaction that results in erythema, edema, and cellular infiltration—especially infiltration of antigen presenting cells (APCs), eosinophils, activated CD4+ T cells, and cytokine-expressing Th2 cells.

To prepare a mouse model for DTH, six cohorts (5 mice per cohort) of 6-8 week old C57Bl/6 mice were obtained from Taconic Biosciences (Germantown, N.Y.). Mice were sensitized on day 0 by four subcutaneous (s.c.) injections at four sites on the back (upper and lower) with 100 μg Keyhole limpet hemocyanin (KLH) emulsified in Complete Freund's Adjuvant (CFA) at a ratio of 1:1 in 200 μl. Cutaneous DTH was elicited on the ear on day 8 by challenging the mice with an intradermal injection of 10 μg of KLH in 10 μl of 0.01% DMSO in saline on the right ear. As a control, the left ear received 10 μl of 0.01% DMSO in saline only. The DTH response, as indicated by ear swelling, was determined by measuring the ear thickness prior to and at various time points post-challenge using a Mitutoyo micrometer. The ear thickness was measured before intradermal challenge as the baseline level for each individual animal. The ear thickness was also measured two times after intradermal challenge, at approximately 24 hours and 48 hours (i.e., days 9 and 10, respectively).

Each cohort of mice were administered once every day for 9 days as follows:

(i) Oral administration of anaerobic PBS (vehicle control);

(ii) Intraperitoneal administration of dexamethasone at 1 mg/kg (positive control);

(iii) Oral administration of 1×10⁹ CFU Prevotella histicola biomass cultured in BM1 media (no B12) comprising 1 g/L spirulina (V3);

(iv) Oral administration of 1×10⁹ CFU Prevotella histicola biomass cultured in BM1 media comprising 1 g/L spirulina (V4);

(v) Oral administration of 1×10⁹ CFU Prevotella histicola biomass cultured in SPYG1 media comprising 1 g/L spirulina (V1); or

(vi) Oral administration of 10 mg powder of Prevotella histicola cultured in growth media comprising hemoglobin.

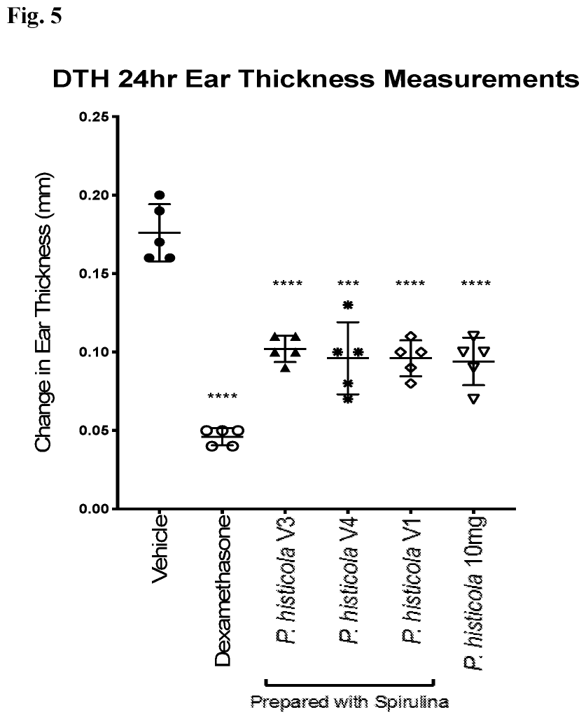

As can be seen in FIG. 5, Prevotella histicola (Prevotella Strain B 50329 (NRRL accession number B 50329)) cultured in the presence of spirulina were just as efficacious as those cultured in the presence of hemoglobin in reducing the DTH response as evidenced by the reduction in ear thickness. Accordingly, spirulina facilitates the production of hemoglobin-dependent bacteria (in the absence of hemoglobin) that are functionally equivalent to the hemoglobin-dependent bacteria cultured in the presence of hemoglobin.

### Example 6: Spirulina can Substitute for Hemoglobin to Facilitate the Growth of Fournierella and Parabacteroides Bacteria

The following hemoglobin-dependent bacteria were cultured in growth media with or without spirulina: Fournierella Strain A, Fournierella Strain B, and Parabacteroides Strain A. The hemoglobin-dependent bacteria were grown in growth media comprising the components listed in Table 6.

TABLE 6
Growth Media SPY

| | g/L |
|---|---|
| Component | SPY |
| Yeast Extract 19512 Organotechnie S.A.S. | 10 |
| Soy Peptone A2 SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |

Carbon sources used were N-acetyl-glucosamine (NAG) or Glucose (Glu) at a final concentration of 5 g/L. Hemoglobin solution was used at a final concentration of 0.02 g/L, added from a 1% stock solution in 0.01M NaOH. Spirulina solution was used at a final concentration of 1 g/L, added from a 5% stock solution in 0.01M NaOH.

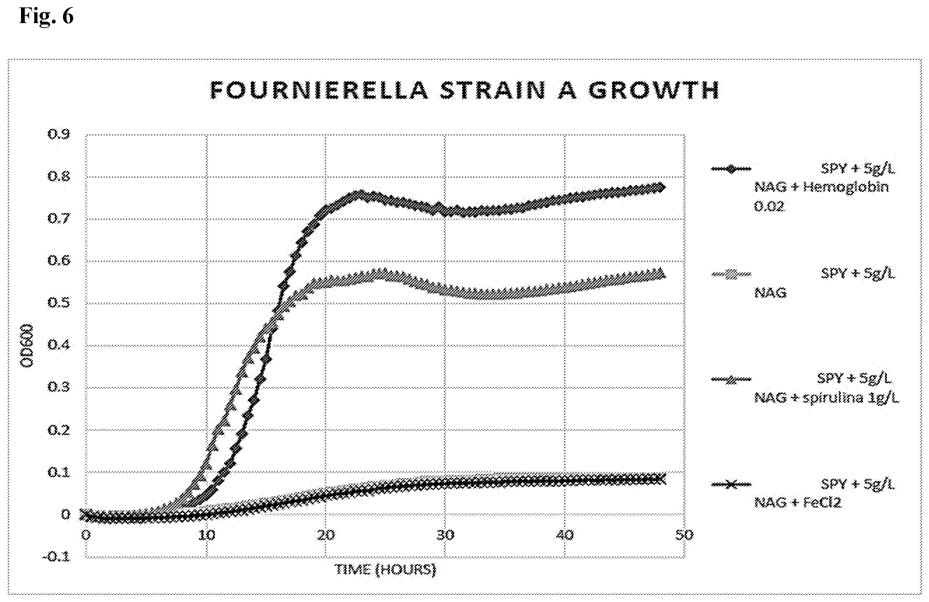

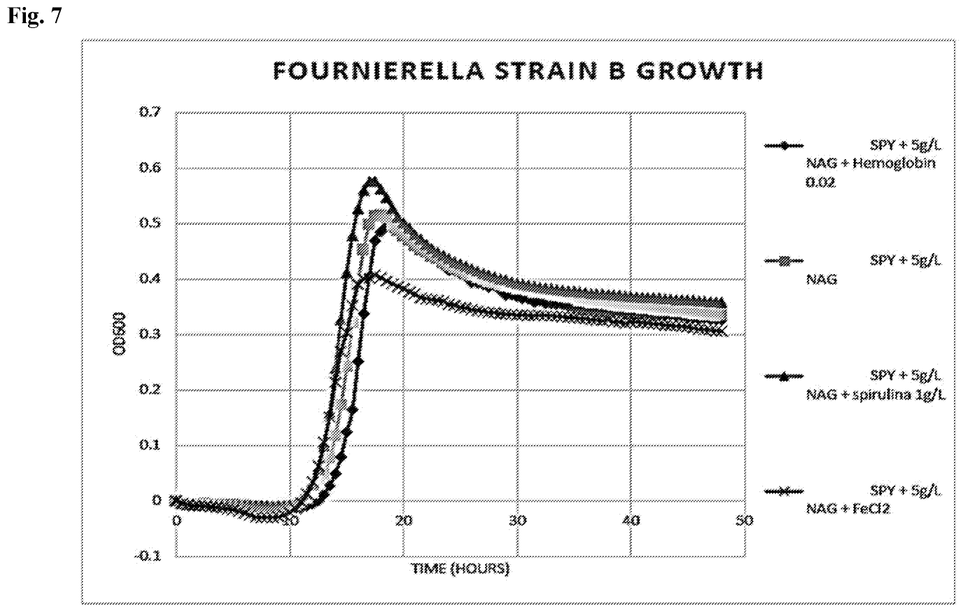

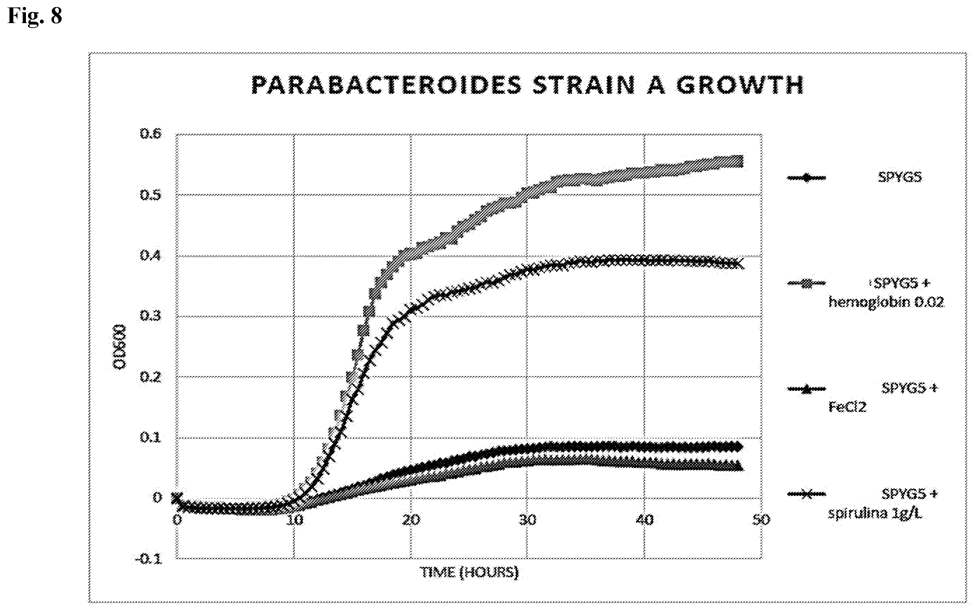

As shown in FIG. 6-FIG. 8, the growth media comprising spirulina supported the growth of each of these hemoglobin-dependent bacteria in the absence of hemoglobin or a derivative thereof. Spirulina restored growth to comparable levels as with growth in hemoglobin containing media for Fournierella Strain A and Parabacteroides Strain A (FIG. 6 and FIG. 8). Fournierella Strain B showed slight improvement in growth with spirulina in these conditions, also comparable with the growth using hemoglobin.

### Example 7: Use of Spirulina to Replace Hemoglobin for Other Hemoglobin-Dependent Bacteria

Microbes tested in these experiments were Parabacteroides Strain B, Faecalibacterium Strain A, Bacteroides Strain A, and Alistipes Strain A.

Parabacteroides Strain B is of the same genus (Parabacteroides) as Parabacteroides Strain A, but is of a different species of the genus.

Alistipes Strain A tested in an endpoint study to determine best growth conditions.

Base medium used to test these microbes was SPY or PM11 with the following compositions:

TABLE 7
Growth Media

| | g/L |
|---|---|
| Component | SPY |
| Yeast Extract 19512 Organotechnie S.A.S. | 10 |
| Soy Peptone A2 SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |

TABLE 8
Growth Media

| | g/L |
|---|---|
| Component | PM11 |
| Yeast Extract 19512 | 10 |
| Soy Peptone E110 19885 | 10 |
| Soy Peptone A3 SC 19685 | 10 |
| Tri-sodium citrate | 5 |
| Dipotassium Phosphate K2HPO4 | 5.03 |
| Monopotassium Phosphate KH2PO4 | 2.87 |
| Magnesium chloride | 0.5 |
| Manganese chloride | 0.1 |
| L-Cysteine-HCl | 0.5 |
| FeSO4 | 0.05 |

Carbon source used was glucose (Glu) at a final concentration of 5 g/L (Glu5) or 10 g/L (Glu10).

Hemoglobin solution was used at a final concentration of 0.2 g/L, added from a 1% stock solution in 0.01M NaOH.

Spirulina solution was used at a final concentration of 1 g/L or 2 g/L, added from a 5% stock solution in 0.01M NaOH.

Growth dynamics curves are derived from kinetic growth tests performed in a 96-well format on a plate reader in anaerobic conditions.

Endpoint test was performed in anaerobic conditions with 3, OD600 measuring points to determine the best growth conditions.

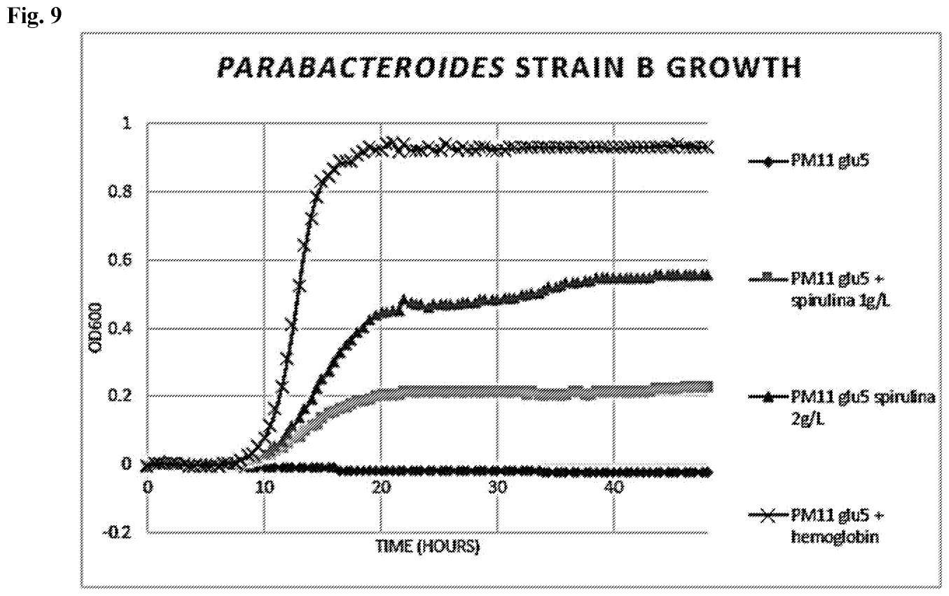

As shown in FIG. 9, Parabacteroides strain B growth is partially restored by addition of spirulina in comparison to hemoglobin. No growth is observed without addition of hemoglobin or spirulina, making this strain hemoglobin dependent. Addition of 1 g/L spirulina restores growth partially, 2 g/L spirulina has increased the growth at least twice, potentially increasing the spirulina concentration above 2 g/L will lead to growth equivalent to that with hemoglobin.

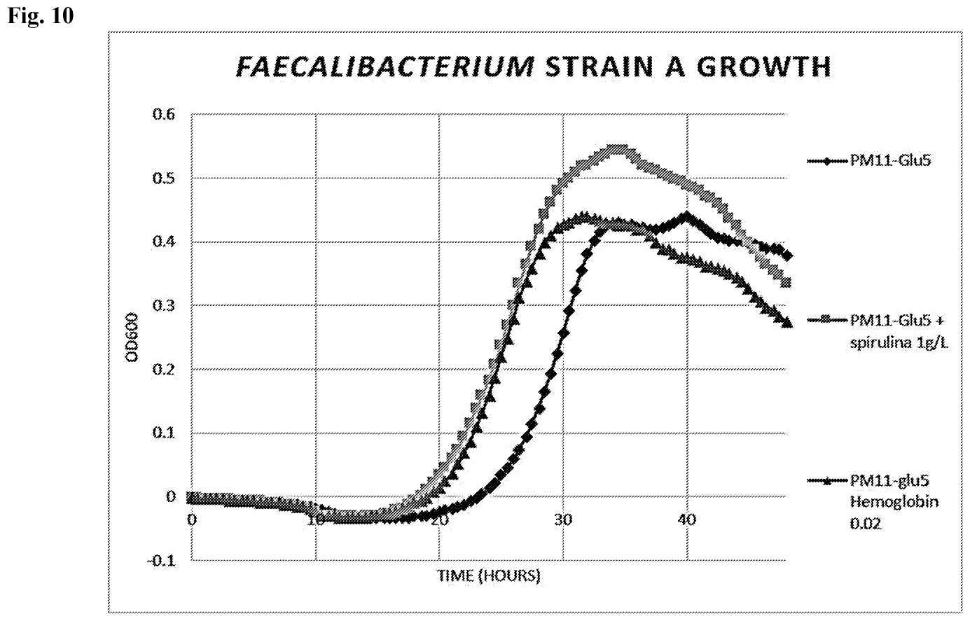

As shown in FIG. 10, Faecalibacterium Strain A growth in the presence of spirulina is equal to or better than growth in hemoglobin containing media. The lag phase is shortened and is similar to that in media with hemoglobin and the optical density is even higher than in the media with hemoglobin.

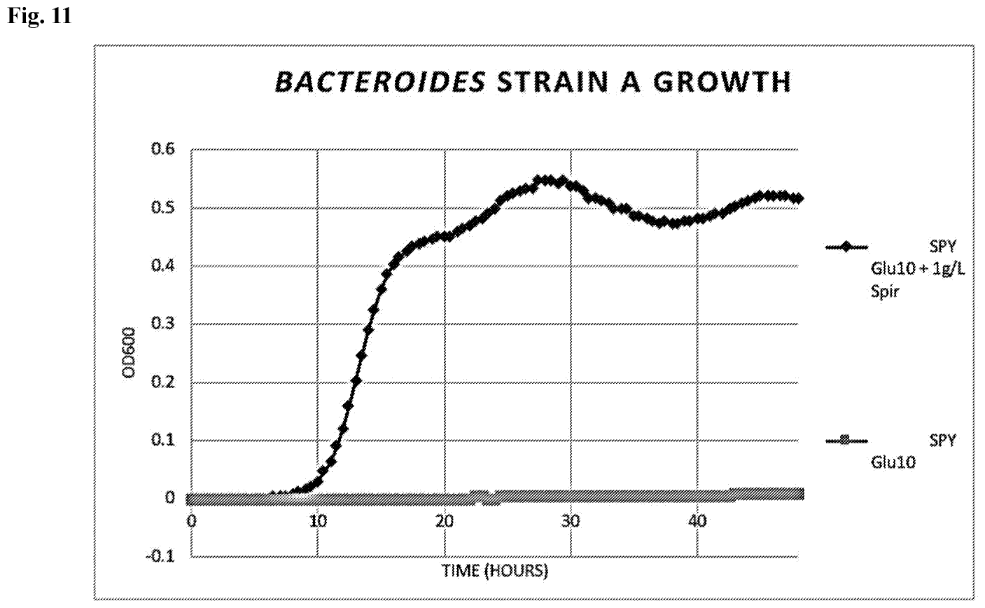

As shown in FIG. 11, Bacteroides Strain A growth is supported with the addition of spirulina, without spirulina the strain does not grow.

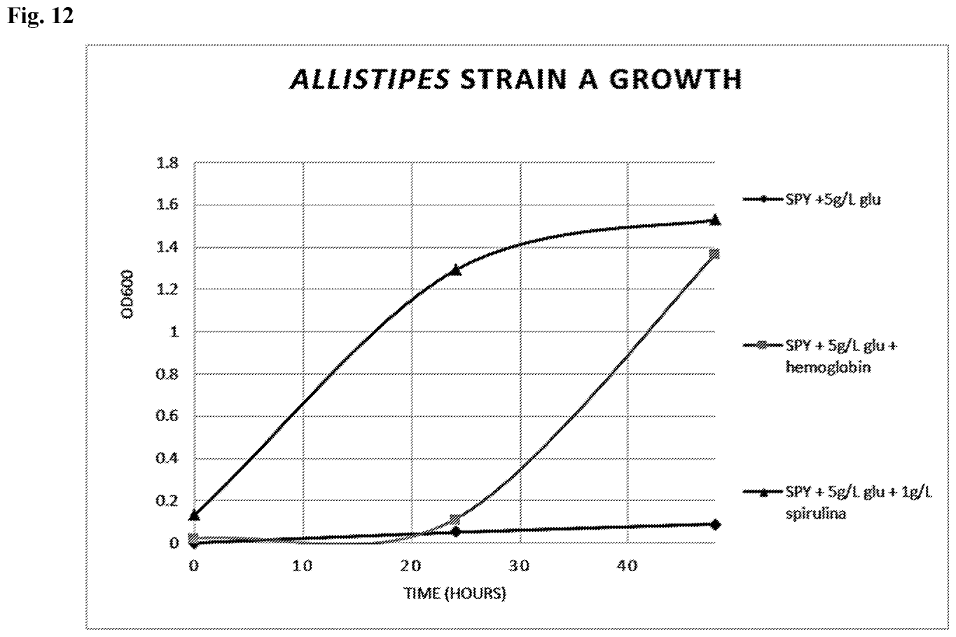

As shown in FIG. 12, Alistipes Strain A growth is better in the medium containing spirulina than in the medium containing hemoglobin.

### Example 8: Use of Spirulina to Replace Hemoglobin for Prevotella Strain C

Another hemoglobin-dependent bacteria, Prevotella Strain C (PTA-126140), was cultured as described in Example 2 in the media according to Table 9A in the presence of spirulina. Spirulina supported the growth of the hemoglobin-dependent Prevotella Strain C (data not shown).

TABLE 9A
Exemplary Growth Media (SPYG)

| | g/L |
|---|---|
| Component | SPYG1 |
| Glucose | 10 |
| Yeast Extract 19512 Organotechnie S.A.S. | 10 |
| Soy Peptone A2 SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |
| Spirulina (Earthrise) | 1 |
| Antifoam | 0.2 ml |

To make 1 L of media, the media components are prepared in 4 different solutions (Solutions 1-4) that are later combined.

### 1. Solution 1

TABLE 9B
Solution 1

| Solution 1 (SPY base): | g/L |
|---|---|
| Yeast Extract 19512 Organotechnie S.A.S. | 10 |
| Soy Peptone A2 SC 19649 Organotechnie S.A.S. | 10 |
| Soy Peptone E110 19885 Organotechnie S.A.S. | 10 |
| Dipotassium Phosphate K2HPO4 | 2.5 |

The components of Solution 1 in Table 9B are dissolved in distilled water, and the volume is adjusted to the final volume of 960 mL. The solution is autoclaved at 121° C. for 30 minutes.

### 2. Solution 2

TABLE 9C
Solution 2

| Solution 2 100X: | For 100 ml |
|---|---|
| L-Cysteine-HCl | 5 g |

5 g of L-Cysteine-HCl is added to 100 mL of distilled water, and is mixed until L-Cysteine-HCl is dissolved. The solution may be mildly heated to facilitate dissolution. The solution is autoclaved at 121° C. for 30 minutes.

### 3. Solution 3

TABLE 9D
Solution 3

| Solution 3 (Glucose) 50x (50%): | For 100 ml |
|---|---|
| Glucose | 50 g |

50 g of glucose is dissolved in distilled water, and the final volume is adjusted to 100 mL. The solution is autoclaved at 121° C. for 30 minutes.

### 4. Solution 4

TABLE 9E
Solution 4
Solution 4: Spirulina 5%

| Components | For 500 ml | |
|---|---|---|
| Sodium Hydroxide (10N stock) | 0.5 | mL |
| Spirulina | 25 | g |

25 g of spirulina powder is added to water and sodium hydroxide, and is stirred until dissolved. Some shaking may be necessary to facilitate resuspension. Once resuspended in solution, the suspension is filtered using a 1 μm filter. The filtered solution is autoclaved at 121° C. for 30 minutes.

The media is finalized by combining all the necessary components as shown in Table 9F in a biosafety cabinet:

TABLE 9F
SPYG Media

| | For 1 L | |
|---|---|---|
| Component | SPYG | |
| Solution 1 (SPY base) | 960 | ml |
| Solution 2 (L-cysteine-HCl) 100x | 10 | ml |
| Solution 3 (Glucose) 50x | 20 | ml |
| Solution 4 (Spirulina) (5%) | 20 | ml |

The complete media is degassed before inoculation with Prevotella.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of culturing hemoglobin-dependent bacteria, the method comprising incubating the hemoglobin-dependent bacteria in a growth medium that comprises a hemoglobin substitute, wherein the hemoglobin substitute is a cyanobacteria, a cyanobacteria component, a cyanobacteria biomass, a green algae, a green algae component, or a green algae biomass.

2. The method of claim 1, wherein the hemoglobin substitute is a cyanobacteria, a cyanobacteria biomass, or a cyanobacteria component.

3. The method of claim 2, wherein the cyanobacteria is of the order Oscillatoriales.

4. The method of claim 2, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

5. The method of claim 4, wherein the cyanobacteria is of the genus Arthrospira.

6. The method of claim 5, wherein the cyanobacteria is Arthrospira platensis and/or Arthrospira maxima.

7. The method of any one of claims 2 to 6, wherein the hemoglobin substitute is a cyanobacteria.

8. The method of any one of claims 2 to 6, wherein the hemoglobin substitute is a cyanobacteria biomass.

9. The method of claim 8, wherein the cyanobacteria biomass is spirulina.

10. The method of any one of claims 2 to 6, wherein the hemoglobin substitute is a cyanobacteria component.

11. The method of claim 10, wherein the cyanobacteria component is a spirulina component.

12. The method of claim 11, wherein the spirulina component is a soluble spirulina component.

13. The method of claim 1, wherein the hemoglobin substitute is a green algae, a green algae component, or a green algae biomass.

14. The method of claim 13, wherein the green algae is of the order Chlorellales.

15. The method of claim 14, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

16. The method of any one of claims 13 to 15, wherein the hemoglobin substitute is a green algae.

17. The method of any one of claims 13 to 15, wherein the hemoglobin substitute is a green algae biomass.

18. The method of any one of claims 13 to 15, wherein the hemoglobin substitute is a green algae component.

19. The method of any one of claims 1-18, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

20. The method of any one of claims 1-18, wherein the hemoglobin-dependent bacteria are of the genus Prevotella.

21. The method of claim 20, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

22. The method of claim 20, wherein the hemoglobin-dependent bacteria are of the species Prevotella histicola.

23. The method of claim 20, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

24. The method of claim 20, wherein the Prevotella comprise at least 99.5% genomic, 16S and/or CRISPR sequence identity to the nucleotide sequence of the Prevotella Strain B 50329 (NRRL accession number B 50329) or Prevotella Strain C (ATTC Deposit Number PTA-126140).

25. The method of claim 20, wherein the Prevotella are Prevotella Strain B 50329 (NRRL accession number B 50329) or Prevotella Strain C (ATTC Deposit Number PTA-126140).

26. The method of any one of claims 20-25, wherein the hemoglobin-dependent bacteria are a strain of Prevotella bacteria comprising one or more proteins listed in Table 1.

27. The method of any one of claims 20-26, wherein the hemoglobin-dependent bacteria are from a strain of Prevotella substantially free of a protein listed in Table 2.

28. The method of any one of claims 1-27, wherein the hemoglobin substitute is able to substitute for hemoglobin in a growth medium to facilitate growth of hemoglobin-dependent bacteria.

29. The method of any one of claims 1-28, wherein the growth medium does not comprise hemoglobin or a derivative thereof.

30. The method of any one of claims 1-29, wherein the growth medium does not comprise animal products.

31. The method of any one of claims 1-30, wherein the hemoglobin-dependent bacteria grow at an increased rate in the growth medium comprising the hemoglobin substitute compared to the rate at which the hemoglobin-dependent bacteria grow in the same growth medium but without the hemoglobin substitute.

32. The method of claim 31, wherein the rate at which the hemoglobin-dependent bacteria grow in the growth medium comprising the hemoglobin substitute is at least 50% higher than the rate at which the hemoglobin-dependent bacteria grow in the same growth medium but without the hemoglobin substitute.

33. The method of claim 31, wherein the rate at which the hemoglobin-dependent bacteria grow in the growth medium comprising the hemoglobin substitute is at least 100% higher than the rate at which the hemoglobin-dependent bacteria grow in the same growth medium but without the hemoglobin substitute.

34. The method of claim 31, wherein the rate at which the hemoglobin-dependent bacteria grow in the growth medium comprising the hemoglobin substitute is 200% to 400% higher than the rate at which the hemoglobin-dependent bacteria grow in the same growth medium but without the hemoglobin substitute.

35. The method of claim 31, wherein the rate at which the hemoglobin-dependent bacteria grow in the growth medium comprising the hemoglobin substitute is at least 300% higher than the rate at which the hemoglobin-dependent bacteria grow in the same growth medium but without the hemoglobin substitute.

36. The method of any one of claims 1-35, wherein the hemoglobin-dependent bacteria grow to a higher cell density in the growth medium comprising the hemoglobin substitute, compared to the cell density to which the hemoglobin-dependent bacteria grow in the same growth medium but without the hemoglobin substitute.

37. The method of claim 36, wherein the hemoglobin-dependent bacteria grow to a cell density in the growth medium comprising the hemoglobin substitute that is at least 50% higher than the cell density to which the hemoglobin-dependent bacteria grow in the same growth medium but without the hemoglobin substitute.

38. The method of claim 36, wherein the hemoglobin-dependent bacteria grow to a cell density in the growth medium comprising the hemoglobin substitute that is at least 100% higher than the cell density to which the hemoglobin-dependent bacteria grow in the same growth medium but without the hemoglobin substitute.

39. The method of claim 36, wherein the hemoglobin-dependent bacteria grow to a cell density in the growth medium comprising the hemoglobin substitute that is at 200% to 400% higher than the cell density to which the hemoglobin-dependent bacteria grow in the same growth medium but without the hemoglobin substitute.

40. The method of claim 36, wherein the hemoglobin-dependent bacteria grow to a cell density in the growth medium comprising the hemoglobin substitute that is at least 300% higher than the cell density to which the hemoglobin-dependent bacteria grow in the same growth medium but without the hemoglobin substitute.

41. The method of any one of claims 1-40, wherein the method comprises incubating the hemoglobin-dependent bacteria under an anaerobic atmosphere comprising greater than 1% CO₂.

42. The method of claim 41, wherein the anaerobic atmosphere comprises at least 10% CO₂.

43. The method of claim 41, wherein the anaerobic atmosphere comprises at least 20% CO₂.

44. The method of claim 41, wherein the anaerobic atmosphere comprises from 10% to 40% CO₂.

45. The method of claim 41, wherein the anaerobic atmosphere comprises from 20% to 30% CO₂.

46. The method of claim 41, wherein the anaerobic atmosphere comprises about 25% CO₂.

47. The method of any one of claims 41-46, wherein the anaerobic atmosphere consists essentially of CO₂ and N₂.

48. The method of claim 41 wherein the anaerobic atmosphere comprises about 25% CO₂ and about 75% N₂.

49. The method of claim 41, wherein the method comprises the steps of
a) purging a bioreactor with an anaerobic gaseous mixture comprising greater than 1% CO₂, and
b) incubating the hemoglobin-dependent bacteria in the bioreactor purged in step a).

50. The method of claim 49, wherein the anaerobic gaseous mixture comprises at least 10% CO₂.

51. The method of claim 49, wherein the anaerobic gaseous mixture comprises at least 20% CO₂.

52. The method of claim 49, wherein the anaerobic gaseous mixture comprises from 10% to 40% CO₂.

53. The method of claim 49, wherein the anaerobic gaseous mixture comprises from 20% to 30% CO₂.

54. The method of claim 49, wherein the anaerobic gaseous mixture comprises about 25% CO₂.

55. The method of any one of claims 49-54, wherein the anaerobic gaseous mixture consists essentially of CO₂ and N₂.

56. The method of claim 49, wherein the anaerobic gaseous mixture comprises about 25% CO₂ and about 75% N₂.

57. The method of any one of claims 49-56, wherein the bioreactor is an about 1 L, about 20 L, about 3,500 L, or about 20,000 L bioreactor.

58. The method of any one of claims 49-57, wherein the method further comprises the step of inoculating a growth medium with hemoglobin-dependent bacteria, wherein the inoculation step precedes step b).

59. The method of claim 58, wherein the volume of hemoglobin-dependent bacteria is about 0.1% v/v of the growth medium.

60. The method of claim 58, wherein the growth medium is about 1 L in volume.

61. The method of claim 58, wherein the volume of hemoglobin-dependent bacteria is about 1 mL.

62. The method of any one of claims 49-61, wherein the hemoglobin-dependent bacteria is incubated for 10-24 hours.

63. The method of claim 62, wherein the hemoglobin-dependent bacteria is incubated for 14 to 16 hours.

64. The method of claim 62 or 63, wherein the method further comprises the step of inoculating about 5% v/v of the cultured bacteria in a growth medium.

65. The method of claim 64, wherein the growth medium is about 20 L in volume.

66. The method of claim 64 or 65, wherein the hemoglobin-dependent bacteria is incubated for 10-24 hours.

67. The method of claim 66, wherein the hemoglobin-dependent bacteria is incubated for 12 to 14 hours.

68. The method of claim 66 or 67, wherein the method further comprises the step of inoculating about 0.5% v/v of the cultured bacteria in a growth medium.

69. The method of claim 68, wherein the growth medium is about 3500 L in volume.

70. The method of claim 68 or 69, wherein the hemoglobin-dependent bacteria is incubated for 10-24 hours.

71. The method of claim 70, wherein the hemoglobin-dependent bacteria is incubated for 12 to 14 hours.

72. The method of any one of claims 1 to 71, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

73. The method of claim 72, wherein the growth medium comprises 5 g/L to 15 g/L yeast extract 19512.

74. The method of claim 72, wherein the growth medium comprises about 10 g/L yeast extract 19512.

75. The method of any one of claims 72 to 74, wherein the growth medium comprises 10 g/L to 15 g/L soy peptone A2SC 19649.

76. The method of claim 75, wherein the growth medium comprises about 12.5 g/L soy peptone A2SC 19649.

77. The method of claim 75, wherein the growth medium comprises about 10 g/L soy peptone A2SC 19649.

78. The method of any one of claims 72 to 77, wherein the growth medium comprises 10 g/L to 15 g/L Soy peptone E110 19885.

79. The method of claim 78, wherein the growth medium comprises about 12.5 g/L Soy peptone E110 19885.

80. The method of claim 78, wherein the growth medium comprises about 10 g/L soy peptone E110 19885.

81. The method of any one of claims 72 to 80, wherein the growth medium comprises 1 g/L to 3 g/L dipotassium phosphate.

82. The method of claim 81, wherein the growth medium comprises about 1.59 g/L dipotassium phosphate.

83. The method of claim 81, wherein the growth medium comprises about 2.5 g/L dipotassium phosphate.

84. The method of any one of claims 72 to 83, wherein the growth medium comprises 0.5 g/L to 1.5 g/L monopotassium phosphate.

85. The method of claim 84, wherein the growth medium comprises about 0.91 g/L monopotassium phosphate.

86. The method of any one of claims 72 to 85, wherein the growth medium comprises 0.1 g/L to 1.0 g/L L-cysteine-HCl.

87. The method of claim 86, wherein the growth medium comprises about 0.5 g/L L-cysteine-HCl.

88. The method of any one of claims 72 to 87, wherein the growth medium comprises 0.1 g/L to 1.0 g/L ammonium chloride.

89. The method of claim 88, wherein the growth medium comprises about 0.5 g/L ammonium chloride.

90. The method of any one of claims 72 to 89, wherein the growth medium comprises 20 g/L to 30 g/L glucidex 21 D.

91. The method of claim 90, wherein the growth medium comprises about 25 g/L glucidex 21 D.

92. The method of any one of claims 72 to 91, wherein the growth medium comprises 5 g/L to 15 g/L glucose.

93. The method of claim 92, wherein the growth medium comprises about 5 g/L glucose or about 10 g/L glucose.

94. The method of any one of claims 1-93, wherein the growth medium comprises at least 0.5 g/L of the hemoglobin substitute.

95. The method of claim 94, wherein the growth medium comprises at least 0.75 g/L the hemoglobin substitute.

96. The method of claim 94, wherein the growth medium comprises at least 1 g/L of the hemoglobin substitute.

97. The method of claim 94, wherein the growth medium comprises about 1 g/L of the hemoglobin substitute.

98. The method of claim 94, wherein the growth medium comprises about 2 g/L of the hemoglobin substitute.

99. The method of any one of claims 1-98, wherein the hemoglobin-dependent bacteria is incubated at a temperature of 35° C. to 39° C.

100. The method of claim 99, wherein the hemoglobin-dependent bacteria is incubated at a temperature of about 37° C.

101. The method of any one of claims 1-100, wherein the growth medium is at a pH of 5.5 to 7.5.

102. The method of claim 101, wherein the growth medium is at a pH of about 6.5.

103. The method of any one of claims 1-102, wherein incubating the hemoglobin-dependent bacteria comprises agitating the growth medium at a RPM of 50 to 300.

104. The method of claim 103, wherein the growth medium is agitated at a RPM of about 150.

105. The method of any one of claims 49-104, wherein the anaerobic gaseous mixture is continuously added during incubation.

106. The method of claim 105, wherein the anaerobic gaseous mixture is added at a rate of about 0.02 vvm.

107. The method of any one of claims 1-106, wherein the method further comprising the step of harvesting the hemoglobin-dependent bacteria when a stationary phase is reached.

108. The method of claim 107, further comprising the step of centrifuging the hemoglobin-dependent bacteria after harvesting to produce a cell paste.

109. The method of claim 108, further comprising diluting the cell paste with a stabilizer solution to produce a cell slurry.

110. The method of claim 109, further comprising the step of lyophilizing the cell slurry to produce a powder.

111. The method of claim 110, further comprising irradiating the powder with gamma radiation.

112. A method of culturing hemoglobin-dependent bacteria, the method comprising (a) adding hemoglobin substitute and hemoglobin-dependent bacteria to a growth medium; and (b) incubating the hemoglobin-dependent bacteria in the growth medium, wherein the hemoglobin substitute is a cyanobacteria, a cyanobacteria component, a cyanobacteria biomass, a green algae, a green algae component, or a green algae biomass.

113. The method of claim 112, wherein the hemoglobin substitute is a cyanobacteria, a cyanobacteria biomass, or a cyanobacteria component.

114. The method of claim 113, wherein the cyanobacteria is of the order Oscillatoriales.

115. The method of claim 113, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

116. The method of claim 115, wherein the cyanobacteria is of the genus Arthrospira.

117. The method of claim 116, wherein the cyanobacteria is Arthrospira platensis and/or Arthrospira maxima.

118. The method of any one of claims 113 to 117, wherein the hemoglobin substitute is a cyanobacteria.

119. The method of any one of claims 113 to 117, wherein the hemoglobin substitute is a cyanobacteria biomass.

120. The method of claim 119, wherein the cyanobacteria biomass is spirulina.

121. The method of any one of claims 113 to 117, wherein the hemoglobin substitute is a cyanobacteria component.

122. The method of claim 121, wherein the cyanobacteria component is a spirulina component.

123. The method of claim 122, wherein the spirulina component is a soluble spirulina component.

124. The method of claim 112, wherein the hemoglobin substitute is a green algae, a green algae component, or a green algae biomass.

125. The method of claim 124, wherein the green algae is of the order Chlorellales.

126. The method of claim 125, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

127. The method of any one of claims 124 to 126, wherein the hemoglobin substitute is a green algae.

128. The method of any one of claims 124 to 126, wherein the hemoglobin substitute is a green algae biomass.

129. The method of any one of claims 124 to 126, wherein the hemoglobin substitute is a green algae component.

130. The method of claim 112-129, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

131. The method of claim 112-129, wherein the hemoglobin-dependent bacteria are of the genus Prevotella.

132. The method of claim 131, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

133. The method of claim 131, wherein the hemoglobin-dependent bacteria are of the species Prevotella histicola.

134. The method of claim 131, wherein the Prevotella comprise at least 90% genomic, 16S and/or CRISPR sequence identity to the nucleotide sequence of the Prevotella Strain B 50329 (NRRL accession number B 50329) or Prevotella Strain C (ATTC Deposit Number PTA-126140).

135. The method of claim 131, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

136. The method of claim 131, wherein the Prevotella are Prevotella Strain B 50329 (NRRL accession number B 50329) or Prevotella Strain C (ATTC Deposit Number PTA-126140).

137. The method of any one of claims 131-136, wherein the hemoglobin-dependent bacteria are a strain of Prevotella bacteria comprising one or more proteins listed in Table 1.

138. The method of any one of claims 131-137, wherein the hemoglobin-dependent bacteria are a strain of Prevotella substantially free of a protein listed in Table 2.

139. The method of any one of claims 112-138, wherein the hemoglobin substitute is able to substitute for hemoglobin in a growth medium to facilitate growth of hemoglobin-dependent bacteria.

140. The method of any one of claims 112-139, wherein the growth medium does not comprise hemoglobin or a derivative thereof.

141. The method of any one of claims 112-140, wherein the growth medium does not comprise animal products.

142. The method of any one of claims 112-141, wherein the hemoglobin-dependent bacteria grow at an increased rate in the growth medium comprising the hemoglobin substitute compared to the rate at which the hemoglobin-dependent bacteria grow in the same growth medium but without the hemoglobin substitute.

143. The method of claim 142, wherein the rate at which the hemoglobin-dependent bacteria grow in the growth medium comprising the hemoglobin substitute is at least 50% higher than the rate at which the hemoglobin-dependent bacteria grow in the same growth medium but without the hemoglobin substitute.

144. The method of claim 142, wherein the rate at which the hemoglobin-dependent bacteria grow in the growth medium comprising the hemoglobin substitute is at least 100% higher than the rate at which the hemoglobin-dependent bacteria grow in the same growth medium but without the hemoglobin substitute.

145. The method of claim 142, wherein the rate at which the hemoglobin-dependent bacteria grow in the growth medium comprising the hemoglobin substitute is 200% to 400% higher than the rate at which the hemoglobin-dependent bacteria grow in the same growth medium but without the hemoglobin substitute.

146. The method of claim 142, wherein the rate at which the hemoglobin-dependent bacteria grow in the growth medium comprising the hemoglobin substitute is at least 300% higher than the rate at which the hemoglobin-dependent bacteria grow in the same growth medium but without the hemoglobin substitute.

147. The method of any one of claims 112-146, wherein the hemoglobin-dependent bacteria grow to a higher cell density in the growth medium comprising the hemoglobin substitute, compared to the cell density to which the hemoglobin-dependent bacteria grow in the same growth medium but without the hemoglobin substitute.

148. The method of claim 147, wherein the hemoglobin-dependent bacteria grow to a cell density in the growth medium comprising the hemoglobin substitute that is at least 50% higher than the cell density to which the hemoglobin-dependent bacteria grow in the same growth medium but without the hemoglobin substitute.

149. The method of claim 147, wherein the hemoglobin-dependent bacteria grow to a cell density in the growth medium comprising the hemoglobin substitute that is at least 100% higher than the cell density to which the hemoglobin-dependent bacteria grow in the same growth medium but without the hemoglobin substitute.

150. The method of claim 147, wherein the hemoglobin-dependent bacteria grow to a cell density in the growth medium comprising the hemoglobin substitute that is at 200% to 400% higher than the cell density to which the hemoglobin-dependent bacteria grow in the same growth medium but without the hemoglobin substitute.

151. The method of claim 147, wherein the hemoglobin-dependent bacteria grow to a cell density in the growth medium comprising the hemoglobin substitute that is at least 300% higher than the cell density to which the hemoglobin-dependent bacteria grow in the same growth medium but without the hemoglobin substitute.

152. The method of any one of claims 112-151, wherein the method comprises incubating the hemoglobin-dependent bacteria under an anaerobic atmosphere comprising greater than 1% CO₂.

153. The method of claim 152, wherein the anaerobic atmosphere comprises at least 10% CO₂.

154. The method of claim 152, wherein the anaerobic atmosphere comprises at least 20% CO₂.

155. The method of claim 152, wherein the anaerobic atmosphere comprises from 10% to 40% CO₂.

156. The method of claim 152, wherein the anaerobic atmosphere comprises from 20% to 30% CO₂.

157. The method of claim 152, wherein the anaerobic atmosphere comprises about 25% CO₂.

158. The method of any one of claims 152-157, wherein the anaerobic atmosphere consists essentially of CO₂ and N₂.

159. The method of claim 152, wherein the anaerobic atmosphere comprises about 25% CO₂ and about 75% N₂.

160. The method of claim 152, wherein the method comprises the steps of
a) purging a bioreactor with an anaerobic gaseous mixture comprising greater than 1% CO₂, and
b) incubating the hemoglobin-dependent bacteria in the bioreactor purged in step a).

161. The method of claim 160, wherein the anaerobic gaseous mixture comprises at least 10% CO₂.

162. The method of claim 160, wherein the anaerobic gaseous mixture comprises at least 20% CO₂.

163. The method of claim 160, wherein the anaerobic gaseous mixture comprises from 10% to 40% CO₂.

164. The method of claim 160, wherein the anaerobic gaseous mixture comprises from 20% to 30% CO₂.

165. The method of claim 160, wherein the anaerobic gaseous mixture comprises about 25% CO₂.

166. The method of any one of claims 160-165, wherein the anaerobic gaseous mixture consists essentially of CO₂ and N₂.

167. The method of claim 160, wherein the anaerobic gaseous mixture comprises about 25% CO₂ and about 75% N₂.

168. The method of any one of claims 160-167, wherein the bioreactor is an about 1 L, about 20 L, about 3,500 L, or about 20,000 L bioreactor.

169. The method of any one of claims claim 160-168, wherein the method further comprises the step of inoculating a growth medium with hemoglobin-dependent bacteria, wherein the inoculation step precedes step b).

170. The method of claim 169, wherein the volume of hemoglobin-dependent bacteria is about 0.1% v/v of the growth medium.

171. The method of claim 169, wherein the growth medium is about 1 L in volume.

172. The method of claim 169, wherein the volume of hemoglobin-dependent bacteria is about 1 mL.

173. The method of any one of claims 160-172, wherein the hemoglobin-dependent bacteria is incubated for 10-24 hours.

174. The method of claim 173, wherein the hemoglobin-dependent bacteria is incubated for 14 to 16 hours.

175. The method of claim 173 or 174, wherein the method further comprises the step of inoculating about 5% v/v of the cultured bacteria in a growth medium.

176. The method of claim 175, wherein the growth medium is about 20 L in volume.

177. The method of claim 175 or 176, wherein the hemoglobin-dependent bacteria is incubated for 10-24 hours.

178. The method of claim 177, wherein the hemoglobin-dependent bacteria is incubated for 12 to 14 hours.

179. The method of claim 177 or 178, wherein the method further comprises the step of inoculating about 0.5% v/v of the cultured bacteria in a growth medium.

180. The method of claim 179, wherein the growth medium is about 3500 L in volume.

181. The method of claim 179 or 180, wherein the hemoglobin-dependent bacteria is incubated for 10-24 hours.

182. The method of claim 181, wherein the hemoglobin-dependent bacteria is incubated for 12 to 14 hours.

183. The method of any one of claims 112-182, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

184. The method of claim 183, wherein the growth medium comprises 5 g/L to 15 g/L yeast extract 19512.

185. The method of claim 183, wherein the growth medium comprises about 10 g/L yeast extract 19512.

186. The method of any one of claims 183 to 185, wherein the growth medium comprises 10 g/L to 15 g/L soy peptone A2SC 19649.

187. The method of claim 186, wherein the growth medium comprises about 12.5 g/L soy peptone A2SC 19649.

188. The method of claim 186, wherein the growth medium comprises about 10 g/L soy peptone A2SC 19649.

189. The method of any one of claims 183 to 188, wherein the growth medium comprises 10 g/L to 15 g/L Soy peptone E110 19885.

190. The method of claim 189, wherein the growth medium comprises about 12.5 g/L Soy peptone E110 19885.

191. The method of claim 189, wherein the growth medium comprises about 10 g/L soy peptone E110 19885.

192. The method of any one of claims 183 to 191, wherein the growth medium comprises 1 g/L to 3 g/L dipotassium phosphate.

193. The method of claim 192, wherein the growth medium comprises about 1.59 g/L dipotassium phosphate.

194. The method of claim 192, wherein the growth medium comprises about 2.5 g/L dipotassium phosphate.

195. The method of any one of claims 183 to 194, wherein the growth medium comprises 0.5 g/L to 1.5 g/L monopotassium phosphate.

196. The method of claim 195, wherein the growth medium comprises about 0.91 g/L monopotassium phosphate.

197. The method of any one of claims 183 to 196, wherein the growth medium comprises 0.1 g/L to 1.0 g/L L-cysteine-HCl.

198. The method of claim 197, wherein the growth medium comprises about 0.5 g/L L-cysteine-HCl.

199. The method of any one of claims 183 to 198, wherein the growth medium comprises 0.1 g/L to 1.0 g/L ammonium chloride.

200. The method of claim 199, wherein the growth medium comprises about 0.5 g/L ammonium chloride.

201. The method of any one of claims 183 to 200, wherein the growth medium comprises 20 g/L to 30 g/L glucidex 21 D.

202. The method of claim 201, wherein the growth medium comprises about 25 g/L glucidex 21 D.

203. The method of any one of claims 183 to 202, wherein the growth medium comprises 5 g/L to 15 g/L glucose.

204. The method of claim 203, wherein the growth medium comprises about 5 g/L glucose or about 10 g/L glucose.

205. The method of any one of claims 112-204, wherein the growth medium comprises at least 0.5 g/L of the hemoglobin substitute.

206. The method of claim 205, wherein the growth medium comprises at least 0.75 g/L the hemoglobin substitute.

207. The method of claim 205, wherein the growth medium comprises at least 1 g/L of the hemoglobin substitute.

208. The method of claim 205, wherein the growth medium comprises about 1 g/L of the hemoglobin substitute.

209. The method of claim 205, wherein the growth medium comprises about 2 g/L of the hemoglobin substitute.

210. The method of any one of claims 112-209, wherein the hemoglobin-dependent bacteria is incubated at a temperature of 35° C. to 39° C.

211. The method of claim 210, wherein the hemoglobin-dependent bacteria is incubated at a temperature of about 37° C.

212. The method of any one of claims 112-211, wherein the growth medium is at a pH of 5.5 to 7.5.

213. The method of claim 212, wherein the growth medium is at a pH of about 6.5.

214. The method of any one of claims 112-213, wherein incubating the hemoglobin-dependent bacteria comprises agitating the growth medium at a RPM of 50 to 300.

215. The method of claim 214, wherein the growth medium is agitated at a RPM of about 150.

216. The method of any one of claims 160-215, wherein the anaerobic gaseous mixture is continuously added during incubation.

217. The method of claim 216, wherein the anaerobic gaseous mixture is added at a rate of about 0.02 vvm.

218. The method of any one of claims 112-217, wherein the method further comprising the step of harvesting the hemoglobin-dependent bacteria when a stationary phase is reached.

219. The method of claim 218, further comprising the step of centrifuging the hemoglobin-dependent bacteria after harvesting to produce a cell paste.

220. The method of claim 219, further comprising diluting the cell paste with a stabilizer solution to produce a cell slurry.

221. The method of claim 220, further comprising the step of lyophilizing the cell slurry to produce a powder.

222. The method of claim 221, further comprising irradiating the powder with gamma radiation.

223. A bioreactor comprising hemoglobin-dependent bacteria in a growth medium comprising a hemoglobin substitute, wherein the hemoglobin substitute is a cyanobacteria, a cyanobacteria component, a cyanobacteria biomass, a green algae, a green algae component, or a green algae biomass.

224. The bioreactor of claim 223, wherein the hemoglobin substitute is a cyanobacteria, a cyanobacteria biomass, or a cyanobacteria component.

225. The bioreactor of claim 224, wherein the cyanobacteria is of the order Oscillatoriales.

226. The bioreactor of claim 224, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

227. The bioreactor of claim 226, wherein the cyanobacteria is of the genus Arthrospira.

228. The bioreactor of claim 227, wherein the cyanobacteria is Arthrospira platensis and/or Arthrospira maxima.

229. The bioreactor of any one of claims 224 to 228, wherein the hemoglobin substitute is a cyanobacteria.

230. The bioreactor of any one of claims 224 to 228, wherein the hemoglobin substitute is a cyanobacteria biomass.

231. The bioreactor of claim 230, wherein the cyanobacteria biomass is spirulina.

232. The bioreactor of any one of claims 224 to 228, wherein the hemoglobin substitute is a cyanobacteria component.

233. The bioreactor of claim 232, wherein the cyanobacteria component is a spirulina component.

234. The bioreactor of claim 233, wherein the spirulina component is a soluble spirulina component.

235. The bioreactor of claim 223, wherein the hemoglobin substitute is a green algae, a green algae component, or a green algae biomass.

236. The bioreactor of claim 235, wherein the green algae is of the order Chlorellales.

237. The bioreactor of claim 236, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

238. The bioreactor of any one of claims 235 to 237, wherein the hemoglobin substitute is a green algae.

239. The bioreactor of any one of claims 235 to 237, wherein the hemoglobin substitute is a green algae biomass.

240. The bioreactor of any one of claims 235 to 237, wherein the hemoglobin substitute is a green algae component.

241. The bioreactor of any one of claims 223-240, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

242. The bioreactor of any one of claims 223-240, wherein the hemoglobin-dependent bacteria are of the genus Prevotella.

243. The bioreactor of claim 242, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

244. The bioreactor of claim 242, wherein the hemoglobin-dependent bacteria are of the species Prevotella histicola.

245. The bioreactor of claim 242, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

246. The bioreactor of claim 242, wherein the Prevotella comprise at least 99.5% genomic, 16S and/or CRISPR sequence identity to the nucleotide sequence of the Prevotella Strain B 50329 (NRRL accession number B 50329) or Prevotella Strain C (ATTC Deposit Number PTA-126140).

247. The bioreactor of claim 242, wherein the Prevotella are Prevotella Strain B 50329 (NRRL accession number B 50329) or Prevotella Strain C (ATTC Deposit Number PTA-126140).

248. The bioreactor of any one of claims 242-247, wherein the hemoglobin-dependent bacteria are a strain of Prevotella bacteria comprising one or more proteins listed in Table 1.

249. The bioreactor of any one of claims 242-248, wherein the hemoglobin-dependent bacteria are from a strain of Prevotella substantially free of a protein listed in Table 2.

250. The bioreactor of any one of claims 223-249, wherein the hemoglobin substitute is able to substitute for hemoglobin in a growth medium to facilitate growth of hemoglobin-dependent bacteria.

251. The bioreactor of any one of claims 223-250, wherein the growth medium does not comprise hemoglobin or a derivative thereof.

252. The bioreactor of any one of claims 223-251, wherein the growth medium does not comprise animal products.

253. The bioreactor of any one of claims 223-252, wherein the hemoglobin-dependent bacteria grow at an increased rate in the growth medium comprising the hemoglobin substitute compared to the rate at which the hemoglobin-dependent bacteria grow in the same growth medium but without the hemoglobin substitute.

254. The bioreactor of claim 253, wherein the rate at which the hemoglobin-dependent bacteria grow in the growth medium comprising the hemoglobin substitute is at least 50% higher than the rate at which the hemoglobin-dependent bacteria grow in the same growth medium but without the hemoglobin substitute.

255. The bioreactor of claim 253, wherein the rate at which the hemoglobin-dependent bacteria grow in the growth medium comprising the hemoglobin substitute is at least 100% higher than the rate at which the hemoglobin-dependent bacteria grow in the same growth medium but without the hemoglobin substitute.

256. The bioreactor of claim 253, wherein the rate at which the hemoglobin-dependent bacteria grow in the growth medium comprising the hemoglobin substitute is 200% to 400% higher than the rate at which the hemoglobin-dependent bacteria grow in the same growth medium but without the hemoglobin substitute.

257. The bioreactor of claim 253, wherein the rate at which the hemoglobin-dependent bacteria grow in the growth medium comprising the hemoglobin substitute is at least 300% higher than the rate at which the hemoglobin-dependent bacteria grow in the same growth medium but without the hemoglobin substitute.

258. The bioreactor of any one of claims 223-257, wherein the hemoglobin-dependent bacteria grow to a higher cell density in the growth medium comprising the hemoglobin substitute, compared to the cell density to which the hemoglobin-dependent bacteria grow in the same growth medium but without the hemoglobin substitute.

259. The bioreactor of claim 258, wherein the hemoglobin-dependent bacteria grow to a cell density in the growth medium comprising the hemoglobin substitute that is at least 50% higher than the cell density to which the hemoglobin-dependent bacteria grow in the same growth medium but without the hemoglobin substitute.

260. The bioreactor of claim 258, wherein the hemoglobin-dependent bacteria grow to a cell density in the growth medium comprising the hemoglobin substitute that is at least 100% higher than the cell density to which the hemoglobin-dependent bacteria grow in the same growth medium but without the hemoglobin substitute.

261. The bioreactor of claim 258, wherein the hemoglobin-dependent bacteria grow to a cell density in the growth medium comprising the hemoglobin substitute that is at 200% to 400% higher than the cell density to which the hemoglobin-dependent bacteria grow in the same growth medium but without the hemoglobin substitute.

262. The bioreactor of claim 258, wherein the hemoglobin-dependent bacteria grow to a cell density in the growth medium comprising the hemoglobin substitute that is at least 300% higher than the cell density to which the hemoglobin-dependent bacteria grow in the same growth medium but without the hemoglobin substitute.

263. The bioreactor of any one of claims 223-262, wherein the hemoglobin-dependent bacteria are under an anaerobic atmosphere comprising at least about 1% CO₂.

264. The bioreactor of claim 263, wherein the anaerobic atmosphere comprises at least 10% CO₂.

265. The bioreactor of claim 263, wherein the anaerobic atmosphere comprises at least 20% CO₂.

266. The bioreactor of claim 263, wherein the anaerobic atmosphere comprises from 10% to 40% CO₂.

267. The bioreactor of claim 263, wherein the anaerobic atmosphere comprises from 20% to 30% CO₂.

268. The bioreactor of claim 263, wherein the anaerobic atmosphere comprises about 25% CO₂.

269. The bioreactor of any one of claims 263-268, wherein the anaerobic atmosphere consists essentially of CO₂ and N₂.

270. The bioreactor of claim 263, wherein the anaerobic atmosphere comprises about 25% CO₂ and about 75% N₂.

271. The bioreactor of any one of claims claim 263-270, wherein bioreactor is a 1 L, 20 L, 3500 L or 20,000 L bioreactor.

272. The bioreactor of any one of claims 223-271, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

273. The bioreactor of claim 272, wherein the growth medium comprises 5 g/L to 15 g/L yeast extract 19512.

274. The bioreactor of claim 272, wherein the growth medium comprises about 10 g/L yeast extract 19512.

275. The bioreactor of any one of claims 272 to 274, wherein the growth medium comprises 10 g/L to 15 g/L soy peptone A2SC 19649.

276. The bioreactor of claim 275, wherein the growth medium comprises about 12.5 g/L soy peptone A2SC 19649.

277. The bioreactor of claim 275, wherein the growth medium comprises about 10 g/L soy peptone A2SC 19649.

278. The bioreactor of any one of claims 272 to 277, wherein the growth medium comprises 10 g/L to 15 g/L Soy peptone E110 19885.

279. The bioreactor of claim 278, wherein the growth medium comprises about 12.5 g/L Soy peptone E110 19885.

280. The bioreactor of claim 278, wherein the growth medium comprises about 10 g/L soy peptone E110 19885.

281. The bioreactor of any one of claims 272-280, wherein the growth medium comprises 1 g/L to 3 g/L dipotassium phosphate.

282. The bioreactor of claim 281, wherein the growth medium comprises about 1.59 g/L dipotassium phosphate.

283. The bioreactor of claim 281, wherein the growth medium comprises about 2.5 g/L dipotassium phosphate.

284. The bioreactor of any one of claims 272-283, wherein the growth medium comprises 0.5 g/L to 1.5 g/L monopotassium phosphate.

285. The bioreactor of claim 284, wherein the growth medium comprises about 0.91 g/L monopotassium phosphate.

286. The bioreactor of any one of claims 272-285, wherein the growth medium comprises 0.1 g/L to 1.0 g/L L-cysteine-HCl.

287. The bioreactor of claim 286, wherein the growth medium comprises about 0.5 g/L L-cysteine-HCl.

288. The bioreactor of any one of claims 272-287, wherein the growth medium comprises 0.1 g/L to 1.0 g/L ammonium chloride.

289. The bioreactor of claim 288, wherein the growth medium comprises about 0.5 g/L ammonium chloride.

290. The bioreactor of any one of claims 272-289, wherein the growth medium comprises 20 g/L to 30 g/L glucidex 21 D.

291. The bioreactor of claim 290, wherein the growth medium comprises about 25 g/L glucidex 21 D.

292. The bioreactor of any one of claims 272-291, wherein the growth medium comprises 5 g/L to 15 g/L glucose.

293. The bioreactor of claim 292, wherein the growth medium comprises about 5 g/L glucose or about 10 g/L glucose.

294. The bioreactor of any one of claims 223-293, wherein the growth medium comprises at least 0.5 g/L of the hemoglobin substitute.

295. The bioreactor of claim 294, wherein the growth medium comprises at least 0.75 g/L the hemoglobin substitute.

296. The bioreactor of claim 294, wherein the growth medium comprises at least 1 g/L of the hemoglobin substitute.

297. The bioreactor of claim 294, wherein the growth medium comprises about 1 g/L of the hemoglobin substitute.

298. The bioreactor of claim 294, wherein the growth medium comprises about 2 g/L of the hemoglobin substitute.

299. The bioreactor of any one of claims 223-298, wherein the bioreactor is at a temperature of 35° C. to 39° C.

300. The bioreactor of claim 299, wherein the a bioreactor is at a temperature of 37° C.

301. The bioreactor of any one of claims 223-300, wherein the growth medium is at a pH of 5.5 to 7.5.

302. The bioreactor of claim 301, wherein the growth medium is at a pH of about 6.5.

303. A method of culturing hemoglobin-dependent bacteria in the bioreactor of any one of claims 223-302, the method comprises incubating the hemoglobin-dependent bacteria in the bioreactor.

304. The method of claim 303, wherein the hemoglobin-dependent bacteria are incubated in an anaerobic gaseous mixture comprising greater than 1% CO₂.

305. The method of claim 303, wherein the anaerobic gaseous mixture comprises at least 10% CO₂.

306. The method of claim 303, wherein the anaerobic gaseous mixture comprises at least 20% CO₂.

307. The method of claim 303, wherein the anaerobic gaseous mixture comprises from 10% to 40% CO₂.

308. The method of claim 303, wherein the anaerobic gaseous mixture comprises from 20% to 30% CO₂.

309. The method of claim 303, wherein the anaerobic gaseous mixture comprises about 25% CO₂.

310. The method of any one of claims 303-309, wherein the anaerobic gaseous mixture consists essentially of CO₂ and N₂.

311. The method of claim 310, wherein the anaerobic gaseous mixture comprises about 25% CO₂ and about 75% N₂.

312. The method of any one of claims claim 303-311, wherein the method further comprises the step of inoculating the growth medium with the hemoglobin-dependent bacteria prior to incubation.

313. The method of claim 312, wherein the volume of hemoglobin-dependent bacteria inoculated is about 0.1% v/v of the growth medium.

314. The method of claim 312, wherein the growth medium is about 1 L in volume.

315. The method of claim 312, wherein the volume of hemoglobin-dependent bacteria inoculated is about 1 mL.

316. The method of any one of claims 303-315, wherein the hemoglobin-dependent bacteria is cultured for 10-24 hours.

317. The method of claim 316, wherein the hemoglobin-dependent bacteria is incubated for 14 to 16 hours.

318. The method of claim 316 or 317, wherein the method further comprises the step of inoculating about 5% v/v of the cultured bacteria in a growth medium.

319. The method of claim 318, wherein the growth medium is about 20 L in volume.

320. The method of claim 318 or 319, wherein the hemoglobin-dependent bacteria is incubated for 10-24 hours.

321. The method of claim 320, wherein the hemoglobin-dependent bacteria is incubate for 12 to 14 hours.

322. The method of claim 320 or 321, wherein the method further comprises the step of inoculating about 0.5% v/v of the cultured bacteria in a growth medium.

323. The method of claim 322, wherein the growth medium is about 3500 L in volume.

324. The method of claim 322 or 323, wherein the hemoglobin-dependent bacteria is incubated for 10-24 hours.

325. The method of claim 324, wherein the hemoglobin-dependent bacteria is incubated for 12 to 14 hours.

326. The method of any one of claims 303-325, wherein the hemoglobin-dependent bacteria is incubated at a temperature of 35° C. to 39° C.

327. The method of claim 326, wherein the hemoglobin-dependent bacteria is incubated at a temperature of 37° C.

328. The method of any one of claims 303-327, wherein incubating the hemoglobin-dependent bacteria comprises agitating the growth medium at a RPM of 50 to 300.

329. The method of claim 328, wherein the growth medium is agitated at a RPM of 150.

330. The method of any one of claims 303-329, wherein the anaerobic gaseous mixture is continuously added during incubation.

331. The method of claim 330, wherein the anaerobic gaseous mixture is added at a rate of 0.02 vvm.

332. The method of any one of claims 303-331, wherein the method further comprises the step of harvesting the hemoglobin-dependent bacteria when a stationary phase is reached.

333. The method of claim 332, further comprising the step of centrifuging the hemoglobin-dependent bacteria after harvesting to produce a cell paste.

334. The method of claim 333, further comprising diluting the cell paste with a stabilizer solution to produce a cell slurry.

335. The method of claim 334, further comprising the step of lyophilizing the cell slurry to produce a powder.

336. The method of claim 335, further comprising irradiating the powder with gamma radiation.

337. A composition comprising
a) hemoglobin-dependent bacteria, and
b) a growth medium comprising a hemoglobin substitute, wherein the hemoglobin substitute is a cyanobacteria, a cyanobacteria component, a cyanobacteria biomass, a green algae, a green algae component, or a green algae biomass.

338. The composition of claim 337, wherein the hemoglobin substitute is a cyanobacteria, a cyanobacteria biomass, or a cyanobacteria component.

339. The composition of claim 338, wherein the cyanobacteria is of the order Oscillatoriales.

340. The composition of claim 338, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

341. The composition of claim 340, wherein the cyanobacteria is of the genus Arthrospira.

342. The composition of claim 341, wherein the cyanobacteria is Arthrospira platensis and/or Arthrospira maxima.

343. The composition of any one of claims 338 to 342, wherein the hemoglobin substitute is a cyanobacteria.

344. The composition of any one of claims 338 to 342, wherein the hemoglobin substitute is a cyanobacteria biomass.

345. The composition of claim 344, wherein the cyanobacteria biomass is spirulina.

346. The composition of any one of claims 338 to 342, wherein the hemoglobin substitute is a cyanobacteria component.

347. The composition of claim 346, wherein the cyanobacteria component is a spirulina component.

348. The composition of claim 347, wherein the spirulina component is a soluble spirulina component.

349. The composition of claim 337, wherein the hemoglobin substitute is a green algae, a green algae component, or a green algae biomass.

350. The composition of claim 349, wherein the green algae is of the order Chlorellales.

351. The composition of claim 350, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

352. The composition of any one of claims 349 to 351, wherein the hemoglobin substitute is a green algae.

353. The composition of any one of claims 349 to 351, wherein the hemoglobin substitute is a green algae biomass.

354. The composition of any one of claims 349 to 351, wherein the hemoglobin substitute is a green algae component.

355. The composition of any one of claims 337-354, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

356. The composition of any one of claims 337-354, wherein the hemoglobin-dependent bacteria are of the genus Prevotella.

357. The composition of claim 356, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

358. The composition of claim 356, wherein the hemoglobin-dependent bacteria are of the species Prevotella histicola.

359. The composition of claim 356, wherein the Prevotella comprise at least 90% genomic, 16S and/or CRISPR sequence identity to the nucleotide sequence of the Prevotella Strain B 50329 (NRRL accession number B 50329) or Prevotella Strain C (ATTC Deposit Number PTA-126140).

360. The composition of claim 356, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

361. The composition of claim 356, wherein the Prevotella are Prevotella Strain B 50329 (NRRL accession number B 50329) or Prevotella Strain C (ATTC Deposit Number PTA-126140).

362. The composition of any one of claims 356-361, wherein the hemoglobin-dependent bacteria are a strain of Prevotella bacteria comprising one or more proteins listed in Table 1.

363. The composition of any one of claims 356-362, wherein the hemoglobin-dependent bacteria are a strain of Prevotella substantially free of a protein listed in Table 2.

364. The composition of any one of claims 337-363, wherein the hemoglobin substitute is able to substitute for hemoglobin in a growth medium to facilitate growth of hemoglobin-dependent bacteria.

365. The composition of any one of claims 337-364, wherein the growth medium does not comprise hemoglobin or a derivative thereof.

366. The composition of any one of claims 337-365, wherein the growth medium does not comprise animal products.

367. The composition of any one of claims 337-366, wherein the hemoglobin-dependent bacteria grow at an increased rate in the growth medium comprising the hemoglobin substitute compared to the rate at which the hemoglobin-dependent bacteria grow in the same growth medium but without the hemoglobin substitute.

368. The composition of claim 367, wherein the rate at which the hemoglobin-dependent bacteria grow in the growth medium comprising the hemoglobin substitute is at least 50% higher than the rate at which the hemoglobin-dependent bacteria grow in the same growth medium but without the hemoglobin substitute.

369. The composition of claim 367, wherein the rate at which the hemoglobin-dependent bacteria grow in the growth medium comprising the hemoglobin substitute is at least 100% higher than the rate at which the hemoglobin-dependent bacteria grow in the same growth medium but without the hemoglobin substitute.

370. The composition of claim 367, wherein the rate at which the hemoglobin-dependent bacteria grow in the growth medium comprising the hemoglobin substitute is 200% to 400% higher than the rate at which the hemoglobin-dependent bacteria grow in the same growth medium but without the hemoglobin substitute.

371. The composition of claim 367, wherein the rate at which the hemoglobin-dependent bacteria grow in the growth medium comprising the hemoglobin substitute is at least 300% higher than the rate at which the hemoglobin-dependent bacteria grow in the same growth medium but without the hemoglobin substitute.

372. The composition of any one of claims 337-371, wherein the hemoglobin-dependent bacteria grow to a higher cell density in the growth medium comprising the hemoglobin substitute, compared to the cell density to which the hemoglobin-dependent bacteria grow in the same growth medium but without the hemoglobin substitute.

373. The composition of claim 372, wherein the hemoglobin-dependent bacteria grow to a cell density in the growth medium comprising the hemoglobin substitute that is at least 50% higher than the cell density to which the hemoglobin-dependent bacteria grow in the same growth medium but without the hemoglobin substitute.

374. The composition of claim 372, wherein the hemoglobin-dependent bacteria grow to a cell density in the growth medium comprising the hemoglobin substitute that is at least 100% higher than the cell density to which the hemoglobin-dependent bacteria grow in the same growth medium but without the hemoglobin substitute.

375. The composition of claim 372, wherein the hemoglobin-dependent bacteria grow to a cell density in the growth medium comprising the hemoglobin substitute that is at 200% to 400% higher than the cell density to which the hemoglobin-dependent bacteria grow in the same growth medium but without the hemoglobin substitute.

376. The composition of claim 372, wherein the hemoglobin-dependent bacteria grow to a cell density in the growth medium comprising the hemoglobin substitute that is at least 300% higher than the cell density to which the hemoglobin-dependent bacteria grow in the same growth medium but without the hemoglobin substitute.

377. The composition of any one of claims 337-376, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

378. The composition of claim 377, wherein the growth medium comprises 5 g/L to 15 g/L yeast extract 19512.

379. The composition of claim 377, wherein the growth medium comprises about 10 g/L yeast extract 19512.

380. The composition of any one of claims 377-379, wherein the growth medium comprises 10 g/L to 15 g/L soy peptone A2SC 19649.

381. The composition of claim 380, wherein the growth medium comprises about 12.5 g/L soy peptone A2SC 19649.

382. The composition of claim 380, wherein the growth medium comprises about 10 g/L soy peptone A2SC 19649.

383. The composition of any one of claims 377-382, wherein the growth medium comprises 10 g/L to 15 g/L Soy peptone E110 19885.

384. The composition of claim 383, wherein the growth medium comprises about 12.5 g/L Soy peptone E110 19885.

385. The composition of claim 383, wherein the growth medium comprises about 10 g/L soy peptone E110 19885.

386. The composition of any one of claims 377-385, wherein the growth medium comprises 1 g/L to 3 g/L dipotassium phosphate.

387. The composition of claim 386, wherein the growth medium comprises about 1.59 g/L dipotassium phosphate.

388. The composition of claim 386, wherein the growth medium comprises about 2.5 g/L dipotassium phosphate.

389. The composition of any one of claims 377-388, wherein the growth medium comprises 0.5 g/L to 1.5 g/L monopotassium phosphate.

390. The composition of claim 389, wherein the growth medium comprises about 0.91 g/L monopotassium phosphate.

391. The composition of any one of claims 377-390, wherein the growth medium comprises 0.1 g/L to 1.0 g/L L-cysteine-HCl.

392. The composition of claim 391, wherein the growth medium comprises about 0.5 g/L L-cysteine-HCl.

393. The composition of any one of claims 377-392, wherein the growth medium comprises 0.1 g/L to 1.0 g/L ammonium chloride.

394. The composition of claim 393, wherein the growth medium comprises about 0.5 g/L ammonium chloride.

395. The composition of any one of claims 377-394, wherein the growth medium comprises 20 g/L to 30 g/L glucidex 21 D.

396. The composition of claim 395, wherein the growth medium comprises about 25 g/L glucidex 21 D.

397. The composition of any one of claims 377-396, wherein the growth medium comprises 5 g/L to 15 g/L glucose.

398. The composition of claim 397, wherein the growth medium comprises about 5 g/L glucose or about 10 g/L glucose.

399. The composition of any one of claims 337-398, wherein the growth medium comprises at least 0.5 g/L of the hemoglobin substitute.

400. The composition of claim 399, wherein the growth medium comprises at least 0.75 g/L the hemoglobin substitute.

401. The composition of claim 399, wherein the growth medium comprises at least 1 g/L of the hemoglobin substitute.

402. The composition of claim 399, wherein the growth medium comprises about 1 g/L of the hemoglobin substitute.

403. The composition of claim 399, wherein the growth medium comprises about 2 g/L of the hemoglobin substitute.

404. The composition of any one of claims 337-403, wherein the growth medium is at a pH of 5.5 to 7.5.

405. The composition of claim 404, wherein the growth medium is at a pH of about 6.5.

406. A growth medium for use in culturing hemoglobin-dependent bacteria, the growth medium comprising a hemoglobin substitute, wherein the hemoglobin substitute is a cyanobacteria, a cyanobacteria component, a cyanobacteria biomass, a green algae, a green algae component, or a green algae biomass.

407. The growth medium of claim 406, wherein the hemoglobin substitute is a cyanobacteria, a cyanobacteria biomass, or a cyanobacteria component.

408. The growth medium of claim 407, wherein the cyanobacteria is of the order Oscillatoriales.

409. The growth medium of claim 407, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

410. The growth medium of claim 409, wherein the cyanobacteria is of the genus Arthrospira.

411. The growth medium of claim 410, wherein the cyanobacteria is Arthrospira platensis and/or Arthrospira maxima.

412. The growth medium of any one of claims 407 to 411, wherein the hemoglobin substitute is a cyanobacteria.

413. The growth medium of any one of claims 407 to 411, wherein the hemoglobin substitute is a cyanobacteria biomass.

414. The growth medium of claim 413, wherein the cyanobacteria biomass is spirulina.

415. The growth medium of any one of claims 407 to 411, wherein the hemoglobin substitute is a cyanobacteria component.

416. The growth medium of claim 415, wherein the cyanobacteria component is a spirulina component.

417. The growth medium of claim 416, wherein the spirulina component is a soluble spirulina component.

418. The growth medium of claim 406, wherein the hemoglobin substitute is a green algae, a green algae component, or a green algae biomass.

419. The growth medium of claim 418, wherein the green algae is of the order Chlorellales.

420. The growth medium of claim 419, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

421. The growth medium of any one of claims 418 to 420, wherein the hemoglobin substitute is a green algae.

422. The growth medium of any one of claims 418 to 420, wherein the hemoglobin substitute is a green algae biomass.

423. The growth medium of any one of claims 418 to 420, wherein the hemoglobin substitute is a green algae component.

424. The growth medium of claim 406-423, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

425. The growth medium of any one of claims 406-423, wherein the hemoglobin-dependent bacteria are of the genus Prevotella.

426. The growth medium of claim 425, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

427. The growth medium of claim 425, wherein the hemoglobin-dependent bacteria are of the species Prevotella histicola.

428. The growth medium of claim 425, wherein the Prevotella comprise at least 90% genomic, 16S and/or CRISPR sequence identity to the nucleotide sequence of the Prevotella Strain B 50329 (NRRL accession number B 50329) or Prevotella Strain C (ATTC Deposit Number PTA-126140).

429. The growth medium of claim 425, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

430. The growth medium of claim 425, wherein the Prevotella are Prevotella Strain B 50329 (NRRL accession number B 50329) or Prevotella Strain C (ATTC Deposit Number PTA-126140).

431. The growth medium of any one of claims 425-430, wherein the hemoglobin-dependent bacteria are a strain of Prevotella bacteria comprising one or more proteins listed in Table 1.

432. The growth medium of any one of claims 425-431, wherein the hemoglobin-dependent bacteria are a strain of Prevotella substantially free of a protein listed in Table 2.

433. The growth medium of any one of claims 406-432, wherein the hemoglobin substitute is able to substitute for hemoglobin in a growth medium to facilitate growth of hemoglobin-dependent bacteria.

434. The growth medium of any one of claims 406-433, wherein the growth medium does not comprise hemoglobin or a derivative thereof.

435. The growth medium of any one of claims 406-434, wherein the growth medium does not comprise animal products.

436. The growth medium of any one of claims 406-435, wherein the hemoglobin-dependent bacteria grow at an increased rate in the growth medium comprising the hemoglobin substitute compared to the rate at which the hemoglobin-dependent bacteria grow in the same growth medium but without the hemoglobin substitute.

437. The growth medium of claim 436, wherein the rate at which the hemoglobin-dependent bacteria grow in the growth medium comprising the hemoglobin substitute is at least 50% higher than the rate at which the hemoglobin-dependent bacteria grow in the same growth medium but without the hemoglobin substitute.

438. The growth medium of claim 436, wherein the rate at which the hemoglobin-dependent bacteria grow in the growth medium comprising the hemoglobin substitute is at least 100% higher than the rate at which the hemoglobin-dependent bacteria grow in the same growth medium but without the hemoglobin substitute.

439. The growth medium of claim 436, wherein the rate at which the hemoglobin-dependent bacteria grow in the growth medium comprising the hemoglobin substitute is 200% to 400% higher than the rate at which the hemoglobin-dependent bacteria grow in the same growth medium but without the hemoglobin substitute.

440. The growth medium of claim 436, wherein the rate at which the hemoglobin-dependent bacteria grow in the growth medium comprising the hemoglobin substitute is at least 300% higher than the rate at which the hemoglobin-dependent bacteria grow in the same growth medium but without the hemoglobin substitute.

441. The growth medium of any one of claims 406-440, wherein the hemoglobin-dependent bacteria grow to a higher cell density in the growth medium comprising the hemoglobin substitute, compared to the cell density to which the hemoglobin-dependent bacteria grow in the same growth medium but without the hemoglobin substitute.

442. The growth medium of claim 441, wherein the hemoglobin-dependent bacteria grow to a cell density in the growth medium comprising the hemoglobin substitute that is at least 50% higher than the cell density to which the hemoglobin-dependent bacteria grow in the same growth medium but without the hemoglobin substitute.

443. The growth medium of claim 441, wherein the hemoglobin-dependent bacteria grow to a cell density in the growth medium comprising the hemoglobin substitute that is at least 100% higher than the cell density to which the hemoglobin-dependent bacteria grow in the same growth medium but without the hemoglobin substitute.

444. The growth medium of claim 441, wherein the hemoglobin-dependent bacteria grow to a cell density in the growth medium comprising the hemoglobin substitute that is at 200% to 400% higher than the cell density to which the hemoglobin-dependent bacteria grow in the same growth medium but without the hemoglobin substitute.

445. The growth medium of claim 441, wherein the hemoglobin-dependent bacteria grow to a cell density in the growth medium comprising the hemoglobin substitute that is at least 300% higher than the cell density to which the hemoglobin-dependent bacteria grow in the same growth medium but without the hemoglobin substitute.

446. The growth medium of any one of claims 406-445, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

447. The growth medium of claim 446, wherein the growth medium comprises 5 g/L to 15 g/L yeast extract 19512.

448. The growth medium of claim 446, wherein the growth medium comprises about 10 g/L yeast extract 19512.

449. The growth medium of any one of claims 446-448, wherein the growth medium comprises 10 g/L to 15 g/L soy peptone A2SC 19649.

450. The growth medium of claim 449, wherein the growth medium comprises about 12.5 g/L soy peptone A2SC 19649.

451. The growth medium of claim 449, wherein the growth medium comprises about 10 g/L soy peptone A2SC 19649.

452. The growth medium of any one of claims 446-451, wherein the growth medium comprises 10 g/L to 15 g/L Soy peptone E110 19885.

453. The growth medium of claim 452, wherein the growth medium comprises about 12.5 g/L Soy peptone E110 19885.

454. The growth medium of claim 452, wherein the growth medium comprises about 10 g/L soy peptone E110 19885.

455. The growth medium of any one of claims 446-454, wherein the growth medium comprises 1 g/L to 3 g/L dipotassium phosphate.

456. The growth medium of claim 455, wherein the growth medium comprises about 1.59 g/L dipotassium phosphate.

457. The growth medium of claim 455, wherein the growth medium comprises about 2.5 g/L dipotassium phosphate.

458. The growth medium of any one of claims 446-457, wherein the growth medium comprises 0.5 g/L to 1.5 g/L monopotassium phosphate.

459. The growth medium of claim 458, wherein the growth medium comprises about 0.91 g/L monopotassium phosphate.

460. The growth medium of any one of claims 446-459, wherein the growth medium comprises 0.1 g/L to 1.0 g/L L-cysteine-HCl.

461. The growth medium of claim 460, wherein the growth medium comprises about 0.5 g/L L-cysteine-HCl.

462. The growth medium of any one of claims 446-461, wherein the growth medium comprises 0.1 g/L to 1.0 g/L ammonium chloride.

463. The growth medium of claim 462, wherein the growth medium comprises about 0.5 g/L ammonium chloride.

464. The growth medium of any one of claims 446-463, wherein the growth medium comprises 20 g/L to 30 g/L glucidex 21 D.

465. The growth medium of claim 464, wherein the growth medium comprises about 25 g/L glucidex 21 D.

466. The growth medium of any one of claims 446-465, wherein the growth medium comprises 5 g/L to 15 g/L glucose.

467. The growth medium of claim 466, wherein the growth medium comprises about 5 g/L glucose or about 10 g/L glucose.

468. The growth medium of any one of claims 406-467, wherein the growth medium comprises at least 0.5 g/L of the hemoglobin substitute.

469. The growth medium of claim 468, wherein the growth medium comprises at least 0.75 g/L the hemoglobin substitute.

470. The growth medium of claim 468, wherein the growth medium comprises at least 1 g/L of the hemoglobin substitute.

471. The growth medium of claim 468, wherein the growth medium comprises about 1 g/L of the hemoglobin substitute.

472. The growth medium of claim 468, wherein the growth medium comprises about 2 g/L of the hemoglobin substitute.

473. The growth medium of any one of claims 406-472, wherein the growth medium is at a pH of 5.5 to 7.5.

474. The growth medium of claim 473, wherein the growth medium is at a pH of about 6.5.

475. A hemoglobin substitute for use as a substitute for hemoglobin or a derivative thereof in a growth medium for hemoglobin-dependent bacteria, wherein the hemoglobin substitute is a cyanobacteria, a cyanobacteria component, a cyanobacteria biomass, a green algae, a green algae component, or a green algae biomass.

476. The hemoglobin substitute of claim 475, wherein the hemoglobin substitute is a cyanobacteria, a cyanobacteria biomass, or a cyanobacteria component.

477. The hemoglobin substitute of claim 476, wherein the cyanobacteria is of the order Oscillatoriales.

478. The hemoglobin substitute of claim 476, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

479. The hemoglobin substitute of claim 478, wherein the cyanobacteria is of the genus Arthrospira.

480. The hemoglobin substitute of claim 479, wherein the cyanobacteria is Arthrospira platensis and/or Arthrospira maxima.

481. The hemoglobin substitute of any one of claims 476 to 480, wherein the hemoglobin substitute is a cyanobacteria.

482. The hemoglobin substitute of any one of claims 476 to 480, wherein the hemoglobin substitute is a cyanobacteria biomass.

483. The hemoglobin substitute of claim 482, wherein the cyanobacteria biomass is spirulina.

484. The hemoglobin substitute of any one of claims 476 to 480, wherein the hemoglobin substitute is a cyanobacteria component.

485. The hemoglobin substitute of claim 484, wherein the cyanobacteria component is a spirulina component.

486. The hemoglobin substitute of claim 485, wherein the spirulina component is a soluble spirulina component.

487. The hemoglobin substitute of claim 475, wherein the hemoglobin substitute is a green algae, a green algae component, or a green algae biomass.

488. The hemoglobin substitute of claim 487, wherein the green algae is of the order Chlorellales.

489. The hemoglobin substitute of claim 488, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

490. The hemoglobin substitute of any one of claims 487 to 489, wherein the hemoglobin substitute is a green algae.

491. The hemoglobin substitute of any one of claims 487 to 489, wherein the hemoglobin substitute is a green algae biomass.

492. The hemoglobin substitute of any one of claims 487 to 489, wherein the hemoglobin substitute is a green algae component.

493. The hemoglobin substitute of any one of claims 475-492, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

494. The hemoglobin substitute of any one of claims 475-492, wherein the hemoglobin-dependent bacteria are of the genus Prevotella.

495. The hemoglobin substitute of claim 494, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

496. The hemoglobin substitute of claim 494, wherein the hemoglobin-dependent bacteria are of the species Prevotella histicola.

497. The hemoglobin substitute of claim 494, wherein the Prevotella comprise at least 90% genomic, 16S and/or CRISPR sequence identity to the nucleotide sequence of the Prevotella Strain B 50329 (NRRL accession number B 50329) or Prevotella Strain C (ATTC Deposit Number PTA-126140).

498. The hemoglobin substitute of claim 494, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

499. The hemoglobin substitute of claim 494, wherein the Prevotella are Prevotella Strain B 50329 (NRRL accession number B 50329) or Prevotella Strain C (ATTC Deposit Number PTA-126140).

500. The hemoglobin substitute of any one of claims 494-499, wherein the hemoglobin-dependent bacteria are a strain of Prevotella bacteria comprising one or more proteins listed in Table 1.

501. The hemoglobin substitute of any one of claims 494-500, wherein the hemoglobin-dependent bacteria are a strain of Prevotella substantially free of a protein listed in Table 2.

502. A bacterial composition comprising
(a) hemoglobin-dependent bacteria, and
(b) a hemoglobin substitute, wherein the hemoglobin substitute is a cyanobacteria, a cyanobacteria component, a cyanobacteria biomass, a green algae, a green algae component, or a green algae biomass.

503. The bacterial composition of claim 502, wherein the hemoglobin substitute is a cyanobacteria, a cyanobacteria biomass, or a cyanobacteria component.

504. The bacterial composition of claim 503, wherein the cyanobacteria is of the order Oscillatoriales.

505. The bacterial composition of claim 503, wherein the cyanobacteria is of the genus Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudonabaena, Pseudophormidium, Schizothrix, Spirulina, Starria, Symploca, Trichocoleus, Trichodesmium, or Tychonema.

506. The bacterial composition of claim 504, wherein the cyanobacteria is of the genus Arthrospira.

507. The bacterial composition of claim 505, wherein the cyanobacteria is Arthrospira platensis and/or Arthrospira maxima.

508. The bacterial composition of any one of claims 503 to 507, wherein the hemoglobin substitute is a cyanobacteria.

509. The bacterial composition of any one of claims 503 to 507, wherein the hemoglobin substitute is a cyanobacteria biomass.

510. The bacterial composition of claim 509, wherein the cyanobacteria biomass is spirulina.

511. The bacterial composition of any one of claims 503 to 507, wherein the hemoglobin substitute is a cyanobacteria component.

512. The bacterial composition of claim 511, wherein the cyanobacteria component is a spirulina component.

513. The bacterial composition of claim 512, wherein the spirulina component is a soluble spirulina component.

514. The bacterial composition of claim 502, wherein the hemoglobin substitute is a green algae, a green algae component, or a green algae biomass.

515. The bacterial composition of claim 514, wherein the green algae is of the order Chlorellales.

516. The bacterial composition of claim 515, wherein the green algae is of the genus Acanthosphaera, Actinastrum, Apatococcus, Apodococcus, Auxenochlorella, Brandtia, Carolibrandtia, Catena, Chlorella, Chloroparva, Closteriopsis, Compactochlorella, Coronacoccus, Coronastrum, Cylindrocelis, Diacanthos, Dicellula, Dicloster, Dictyosphaerium, Didymogenes, Eomyces, Fissuricella, Follicularia, Geminella, Gloeotila, Golenkiniopsis, Hegewaldia, Helicosporidium, Heynigia, Hindakia, Hormospora, Kalenjinla, Keratococcus, Kermatia, Leptochlorella, Marasphaerium, Marinchlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Siderocelis, Siderocelopsis, or Zoochlorella.

517. The bacterial composition of any one of claims 514 to 516, wherein the hemoglobin substitute is a green algae.

518. The bacterial composition of any one of claims 514 to 516, wherein the hemoglobin substitute is a green algae biomass.

519. The bacterial composition of any one of claims 514 to 516, wherein the hemoglobin substitute is a green algae component.

520. The bacterial composition of any one of claims 502-519, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, or Veillonella.

521. The bacterial composition of any one of claims 502-519, wherein the hemoglobin-dependent bacteria are of the genus Prevotella.

522. The bacterial composition of claim 521, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

523. The bacterial composition of claim 521, wherein the hemoglobin-dependent bacteria are of the species Prevotella histicola.

524. The bacterial composition of claim 521, wherein the Prevotella comprise at least 90% genomic, 16S and/or CRISPR sequence identity to the nucleotide sequence of the Prevotella Strain B 50329 (NRRL accession number B 50329) or Prevotella Strain C (ATTC Deposit Number PTA-126140).

525. The bacterial composition of claim 521, wherein the Prevotella comprise at least 99% genomic, 16S and/or CRISPR sequence identity to the nucleotide sequence of the Prevotella Strain B 50329 (NRRL accession number B 50329) or Prevotella Strain C (ATTC Deposit Number PTA-126140).

526. The bacterial composition of claim 521, wherein the Prevotella are Prevotella Strain B 50329 (NRRL accession number B 50329) or Prevotella Strain C (ATTC Deposit Number PTA-126140).

527. The bacterial composition of any one of claims 521-526, wherein the hemoglobin-dependent bacteria are a strain of Prevotella bacteria comprising one or more proteins listed in Table 1.

528. The bacterial composition of any one of claims 521-527, wherein the hemoglobin-dependent bacteria are a strain of Prevotella substantially free of a protein listed in Table 2.
